SEQUENCE LISTING (32 sequences)

SEQ ID NO: 1 - DNA, 24 bp, Artificial Sequence
  Description of Artificial Sequence Synthetic probe
agcagggccagaaccagctctata

SEQ ID NO: 2 - DNA, 23 bp, Artificial Sequence
  Description of Artificial Sequence Synthetic primer
tgaactgagagtgaagttcagca

SEQ ID NO: 3 - DNA, 22 bp, Artificial Sequence
  Description of Artificial Sequence Synthetic primer
cttcgtcctagattgagctcgt

SEQ ID NO: 4 - DNA, 24 bp, Artificial Sequence
  Description of Artificial Sequence Synthetic probe
ttatagagctggttctggccctgc

SEQ ID NO: 5 - DNA, 21 bp, Artificial Sequence
  Description of Artificial Sequence Synthetic primer
ggatgtgaactgagagtgaag

SEQ ID NO: 6 - DNA, 21 bp, Artificial Sequence
  Description of Artificial Sequence Synthetic primer
tcctctcttcgtcctagattg

SEQ ID NO: 7 - DNA, 25 bp, Artificial Sequence
  Description of Artificial Sequence Synthetic probe
tcttctggaaatcggcagctacagc

SEQ ID NO: 8 - DNA, 21 bp, Artificial Sequence
  Description of Artificial Sequence Synthetic primer
ggcagaaagaaactcctgtat

SEQ ID NO: 9 - DNA, 21 bp, Artificial Sequence
  Description of Artificial Sequence Synthetic primer
cttcactctcagttcacatcc

SEQ ID NO: 10 - DNA, 25 bp, Artificial Sequence
  Description of Artificial Sequence Synthetic probe
tcttctggaaatcggcagctacagc

SEQ ID NO: 11 - DNA, 22 bp, Artificial Sequence
  Description of Artificial Sequence Synthetic primer
ccagtacaaactactcaagagg

SEQ ID NO: 12 - DNA, 20 bp, Artificial Sequence
  Description of Artificial Sequence Synthetic primer
gctgaacttcactctcagtt

SEQ ID NO: 13 - DNA, 29 bp, Artificial Sequence
  Description of Artificial Sequence Synthetic probe
agaaggaggatgtgaactgagagtgaagt

SEQ ID NO: 14 - DNA, 20 bp, Artificial Sequence
  Description of Artificial Sequence Synthetic primer
ctgccgatttccagaagaag

SEQ ID NO: 15 - DNA, 21 bp, Artificial Sequence
  Description of Artificial Sequence Synthetic primer
tcctctcttcgtcctagattg

SEQ ID NO: 16 - DNA, 26 bp, Artificial Sequence
  Description of Artificial Sequence Synthetic probe
aggaggatgtgaactgagagtgaagt

SEQ ID NO: 17 - DNA, 18 bp, Artificial Sequence
  Description of Artificial Sequence Synthetic primer
ctgtagctgccgatttcc

SEQ ID NO: 18 - DNA, 20 bp, Artificial Sequence
  Description of Artificial Sequence Synthetic primer
atcgtactcctctcttcgtc

SEQ ID NO: 19 - DNA, 26 bp, Artificial Sequence
  Description of Artificial Sequence Synthetic probe
aggaggatgtgaactgagagtgaagt

SEQ ID NO: 20 - DNA, 20 bp, Artificial Sequence
  Description of Artificial Sequence Synthetic primer
ctgccgatttccagaagaag

SEQ ID NO: 21 - DNA, 21 bp, Artificial Sequence
  Description of Artificial Sequence Synthetic primer
tcctctcttcgtcctagattg

SEQ ID NO: 22 - DNA, 25 bp, Artificial Sequence
  Description of Artificial Sequence Synthetic probe
agggagagatttgtgtgggcatgac

SEQ ID NO: 23 - DNA, 22 bp, Artificial Sequence
  Description of Artificial Sequence Synthetic primer
tcatctcttgtgggctgtaatc

SEQ ID NO: 24 - DNA, 21 bp, Artificial Sequence
  Description of Artificial Sequence Synthetic primer
tgctggttctctttcactgac

SEQ ID NO: 25 - DNA, 26 bp, Artificial Sequence
  Description of Artificial Sequence Synthetic probe
cctgtcatgcccacacaaatctctcc

SEQ ID NO: 26 - DNA, 22 bp, Artificial Sequence
  Description of Artificial Sequence Synthetic primer
gctgtcatctcttgtgggctgt

SEQ ID NO: 27 - DNA, 21 bp, Artificial Sequence
  Description of Artificial Sequence Synthetic primer
actcatgggagctgctggttc

SEQ ID NO: 28 - DNA, 24 bp, Artificial Sequence
  Description of Artificial Sequence Synthetic probe
ccctggcattgttgtctttgcaga

SEQ ID NO: 29 - DNA, 18 bp, Artificial Sequence
  Description of Artificial Sequence Synthetic primer
ctgtcatgcccacacaaa

SEQ ID NO: 30 - DNA, 22 bp, Artificial Sequence
  Description of Artificial Sequence Synthetic primer
ataaggctatccaaactcatgg

SEQ ID NO: 31 - DNA, 16 bp, Artificial Sequence
  Description of Artificial Sequence Synthetic probe
aatcggcagctacagc

SEQ ID NO: 32 - DNA, 16 bp, Artificial Sequence
  Description of Artificial Sequence Synthetic probe
tttgtgtgggcatgac

(19) United States
(12) Patent Application Publication
(10) Pub. No.: US 2021/0164045 A1
(43) Pub. Date: Jun. 3, 2021

(54) B-CELL MATURATION COMPLEX CAR T CONSTRUCT AND PRIMERS

(71) Applicant: Janssen Biotech, Inc., Horsham, PA (US)

(72) Inventors: Rebecca GEORGE, Downington, PA (US); Dee SHEN, Berkeley Heights, NJ (US)

(21) Appl. No.: 17/005,858

(22) Filed: Aug. 28, 2020

Related U.S. Application Data
(60) Provisional application No. 62/894,663, filed on Aug. 30, 2019.

(51) Int. Cl. C12Q 1/6876; C12N 15/90

(52) U.S. Cl. CPC C12Q 1/6876; C12N 15/907

(57) ABSTRACT

The present invention provides probe and primer sets, and related methods and kits, for generating B-cell maturation antigen chimeric antigen receptor (CAR) T cells. The invention also provides probe and primer sets, and related methods and kits, for performing quantitative polymerase chain reactions to quantitate B-cell maturation antigen CAR transgene integration into a CAR T drug product.

### CROSS-REFERENCE TO RELATED APPLICATION

This application claims the benefit of U.S. Provisional Application Ser. No. 62/894,663; filed 30 Aug. 2019. The entire content of the aforementioned application is incorporated herein by reference.

### INCORPORATION BY REFERENCE OF MATERIAL IN ASCII TEXT FILE

The instant application contains a Sequence Listing which has been submitted electronically in ASCII format and is hereby incorporated by reference in its entirety. Said ASCII copy, created on Aug. 28, 2020, is named JBI6148USNP1_SL.txt and is 7470 bytes in size.

### BACKGROUND

T cell therapy utilizes isolated T cells that have been genetically modified to enhance their specificity for a specific tumor associated antigen. Genetic modification may involve the expression of a chimeric antigen receptor (CAR) or an exogenous T cell receptor to provide new antigen specificity onto the T cell. T cells expressing chimeric antigen receptors (CAR T cells) can induce tumor immunoreactivity. B cell maturation antigen (BCMA) is a molecule expressed on the surface of mature B cells and malignant plasma cells and is a targeted molecule in the treatment of cancer, for example, multiple myeloma. There is a need for better cancer therapies utilizing CAR T cells, in particular, CAR T cells specific for the BCMA tumor associated antigen.

### SUMMARY

The present invention relates to. probes and primers for polymerase chain reaction (PCR), e.g., quantitative PCR. The present invention also relates to kits and methods utilizing the probes and primers described herein for quantitating transgene integration into chimeric antigen receptor (CAR) T cells.

In a first aspect, the invention provides probe and primer sets comprising a probe comprising a nucleotide sequence of SEQ ID NO: 10 and at least one label attached to the probe; a first primer comprising a nucleic acid sequence of SEQ ID NO: 11; and a second primer comprising a nucleic acid sequence of SEQ ID NO: 12 (See Example 1, Table 1).

In another aspect, the invention provides probe and primer sets comprising a probe comprising a nucleotide sequence of SEQ ID NO: 1 and at least one label attached to the probe; a first primer comprising a nucleic acid sequence of SEQ ID NO: 2; and a second primer comprising a nucleic acid sequence of SEQ ID NO: 3 (See Example 1, Table 1).

In another aspect, the invention provides probe and primer sets comprising a probe comprising a nucleotide sequence selected from the group consisting of SEQ ID NO: 1, SEQ ID NO: 4, SEQ ID NO: 7, SEQ ID NO: 10, SEQ ID NO: 13, SEQ ID NO: 16, SEQ ID NO: 19 and combinations thereof, and at least one label attached to the probe; a first primer comprising a nucleic acid sequence selected from the group consisting of SEQ ID NO: 2, SEQ ID NO: 5, SEQ ID NO: 8, SEQ ID NO: 11, SEQ ID NO: 14, SEQ ID NO: 17, SEQ ID NO: 20 and combinations thereof; and a second primer comprising a nucleic acid sequence selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21 and combinations thereof (See Table 1).

In some embodiments, the at least one label comprises a radioactive isotope, an enzyme substrate, a chemiluminescent agent, a fluorophore, a fluorescence quencher, an enzyme, a chemical, or a combination thereof.

In another aspect, the invention provides kits for quantitating transgene integration into a CAR T cell, comprising: a probe comprising a nucleotide sequence of SEQ ID NO: 10 and at least one label attached to the probe; a first primer comprising a nucleic acid sequence of SEQ ID NO: 11; and a second primer comprising a nucleic acid sequence of SEQ ID NO: 12.

In another aspect, the invention provides kits for quantitating transgene integration into a CAR T cell, comprising: a probe comprising a nucleotide sequence of SEQ ID NO: 1 and at least one label attached to the probe; a first primer comprising a nucleic acid sequence of SEQ ID NO: 2; and a second primer comprising a nucleic acid sequence of SEQ ID NO: 3.

In a further aspect, the invention provides kits for quantitating transgene integration into a CAR T cell, comprising: a probe comprising a nucleotide sequence selected from the group consisting of SEQ ID NO: 1, SEQ ID NO: 4, SEQ ID NO: 7, SEQ ID NO: 10, SEQ ID NO: 13, SEQ ID NO: 16, SEQ ID NO: 19 and combinations thereof, and at least one label attached to the probe; a first primer comprising a nucleic acid sequence selected from the group consisting of SEQ ID NO: 2, SEQ ID NO: 5, SEQ ID NO: 8, SEQ ID NO: 11, SEQ ID NO: 14, SEQ ID NO: 17, SEQ ID NO: 20 and combinations thereof; and a second primer comprising a nucleic acid sequence selected from the group consisting of SEQ ID NO: 3, SEQ ID NO:6, SEQ ID NO: 9, SEQ ID NO: 12, SEQ ID NO: 15, SEQ ID NO: 18, SEQ ID NO: 21 and combinations thereof.

In some embodiments of the kits of the invention, the at least one label attached to the probe comprises a radioactive isotope, an enzyme substrate, a chemiluminescent agent, a fluorophore, a fluorescence quencher, an enzyme, a chemical, or a combination thereof.

In one embodiment, the kits of the invention comprise an array that comprises the probe. In some embodiments, the array is a multi-well plate.

In one embodiment, the kits further comprise a human albumin (hALB) probe comprising a nucleic acid sequence of SEQ ID NO: 22 (See Example 2, Table 2, probe from hALB Set 1) and at least one label attached to the hALB probe, a first hALB primer comprising a nucleic acid sequence of SEQ ID NO: 23 (Table 2, forward primer from hALB Set 1), and a second hALB primer comprising a nucleic acid sequence of SEQ ID NO: 24 (Table 2, reverse primer from hALB Set 1). In certain embodiments, the at least one label attached to the hALB probe comprises a radioactive isotope, an enzyme substrate, a chemiluminescent agent, a fluorophore, a fluorescence quencher, an enzyme, a chemical, or a combination thereof.

In another embodiment, the kits further comprise a human albumin (hALB) probe comprising a nucleic acid sequence selected from the group consisting of SEQ ID NO: 22, SEQ ID NO: 25, SEQ ID NO: 28 and combinations thereof, and at least one label attached to the hALB probe, a first hALB primer comprising a nucleic acid sequence selected from the group consisting of SEQ ID NO: 23, SEQ ID NO: 26, SEQ ID NO: 29 and combinations thereof, and a second hALB primer comprising a nucleic acid sequence selected from the group consisting of SEQ ID NO: 24, SEQ ID NO: 27, SEQ ID NO: 30 and combinations thereof (Table 2). In certain embodiments, the at least one label attached to the hALB probe comprises a radioactive isotope, an enzyme substrate, a chemiluminescent agent, a fluorophore, a fluorescence quencher, an enzyme, a chemical, or a combination thereof.

In another aspect, the present invention provides methods for quantitating transgene integration into a CAR T cell, comprising:

amplifying nucleic acids from the CAR T cell with a first CAR primer comprising a nucleic acid sequence of SEQ ID NO: 11 and a second CAR primer comprising a nucleic acid sequence of SEQ ID NO: 12, thereby generating amplified CAR nucleic acids;

amplifying the nucleic acids from the CAR T cell with a first hALB primer comprising a nucleic acid sequence of SEQ ID NO: 23 and a second hALB primer comprising a nucleic acid sequence of SEQ ID NO: 24, thereby generating amplified hALB nucleic acids;

detecting hybridization between the amplified CAR nucleic acids and a CAR probe comprising a nucleotide sequence of SEQ ID NO: 10 via a target signal from at least one label attached to the CAR probe;

detecting hybridization between the amplified hALB nucleic acids and the hALB probe comprising a nucleotide sequence of SEQ ID NO: 22 via a reference signal from at least one label attached to the hALB probe; and

quantitating transgene copy number by comparison of the target signal relative to the reference signal.

In another aspect, the present invention provides methods for quantitating transgene integration into a CAR T cell, comprising:

amplifying nucleic acids from the CAR T cell with a first CAR primer comprising a nucleic acid sequence of SEQ ID NO: 2 and a second CAR primer comprising a nucleic acid sequence of SEQ ID NO: 3, thereby generating amplified CAR nucleic acids;

amplifying the nucleic acids from the CAR T cell with a first hALB primer comprising a nucleic acid sequence of SEQ ID NO: 23 and a second hALB primer comprising a nucleic acid sequence of SEQ ID NO: 24, thereby generating amplified hALB nucleic acids;

detecting hybridization between the amplified CAR nucleic acids and a CAR probe comprising a nucleotide sequence of SEQ ID NO: 1 via a target signal from at least one label attached to the CAR probe;

detecting hybridization between the amplified hALB nucleic acids and the hALB probe comprising a nucleotide sequence of SEQ ID NO: 22 via a reference signal from at least one label attached to the hALB probe; and

quantitating transgene copy number by comparison of the target signal relative to the reference signal.

In another aspect, the invention provides methods for quantitating transgene integration into a chimeric antigen receptor (CAR) T cell, comprising:

contacting nucleic acids from the CAR T cell with a first CAR primer, a second CAR primer, a first hALB primer and a second hALB primer, wherein the first CAR primer comprises a nucleic acid sequence of SEQ ID NO: 11, the second CAR primer comprises a nucleic acid sequence of SEQ ID NO: 12, the first hALB primer comprises a nucleic acid sequence of SEQ ID NO: 23 and the second hALB primer comprises a nucleic acid sequence of SEQ ID NO: 24;

amplifying the CAR nucleic acids with the first CAR primer and second CAR primer, thereby generating amplified CAR nucleic acids;

amplifying hALB nucleic acids with the first hALB primer and second hALB primer, thereby generating amplified hALB nucleic acids;

detecting hybridization between the amplified CAR nucleic acids and a CAR probe comprising a nucleotide sequence of SEQ ID NO: 10 via a target signal from at least one label attached to the CAR probe;

detecting hybridization between the amplified hALB nucleic acids and the hALB probe via a reference signal from at least one label attached to the hALB probe; and

quantitating transgene copy number by comparison of the target signal relative to the reference signal.

In another aspect, the invention provides methods for quantitating transgene integration into a chimeric antigen receptor (CAR) T cell, comprising:

contacting nucleic acids from the CAR T cell with a first CAR primer, a second CAR primer, a first hALB primer and a second hALB primer, wherein the first CAR primer comprises a nucleic acid sequence of SEQ ID NO: 2, the second CAR primer comprises a nucleic acid sequence of SEQ ID NO: 3, the first hALB primer comprises a nucleic acid sequence of SEQ ID NO: 23 and the second hALB primer comprises a nucleic acid sequence of SEQ ID NO: 24;

amplifying the CAR nucleic acids with the first CAR primer and second CAR primer, thereby generating amplified CAR nucleic acids;

amplifying hALB nucleic acids with the first hALB primer and second hALB primer, thereby generating amplified hALB nucleic acids;

detecting hybridization between the amplified CAR nucleic acids and a CAR probe comprising a nucleotide sequence of SEQ ID NO: 1 via a target signal from at least one label attached to the CAR probe;

detecting hybridization between the amplified hALB nucleic acids and the hALB probe via a reference signal from at least one label attached to the hALB probe; and

quantitating transgene copy number by comparison of the target signal relative to the reference signal.

In a further aspect, the present invention provides methods for quantitating transgene integration into a CAR T cell, comprising:

contacting nucleic acids from the CAR T cell with a first CAR primer comprising a nucleic acid sequence selected from the group consisting of SEQ ID NO: 2, SEQ ID NO: 5, SEQ ID NO: 8, SEQ ID NO: 11, SEQ ID NO: 14, SEQ ID NO: 17, SEQ ID NO: 20 and combinations thereof, and contacting the nucleic acids from the CAR T cell with a second CAR primer comprising a nucleic acid sequence selected from the group consisting of SEQ ID NO: 3, SEQ ID NO:6, SEQ ID NO: 9, SEQ ID NO: 12, SEQ ID NO: 15, SEQ ID NO: 18, SEQ ID NO: 21 and combinations thereof;

contacting the nucleic acids from the CAR T cell with a first hALB primer comprising a nucleic acid sequence selected from the group consisting of SEQ ID NO: 23, SEQ ID NO: 26, SEQ ID NO: 29 and combinations thereof, and contacting the nucleic acids from the CAR T cell with a second hALB primer comprising a nucleic acid sequence selected from the group consisting of SEQ ID NO: 24, SEQ ID NO: 27, SEQ ID NO: 30 and combinations thereof;

contacting the nucleic acids from the CAR T cell with a CAR probe, wherein the CAR probe comprises a nucleotide sequence selected from the group consisting of SEQ ID NO: 1, SEQ ID NO: 4, SEQ ID NO: 7, SEQ ID NO: 10, SEQ ID NO: 13, SEQ ID NO: 16, SEQ ID NO: 19 and combinations thereof;

contacting the nucleic acids from the CAR T cell with a hALB probe, wherein the hALB probe comprises a nucleotide sequence selected from the group consisting of SEQ ID NO: 22, SEQ ID NO: 25, SEQ ID NO: 28 and combinations thereof;

amplifying CAR nucleic acids with the first CAR primer and second CAR primer, thereby generating amplified CAR nucleic acid molecules;

amplifying hALB nucleic acids with the first hALB primer and second hALB primer, thereby generating amplified hALB nucleic acid molecules;

detecting hybridization between the amplified CAR nucleic acid molecules and the CAR probe via a target signal from at least one label attached to the CAR probe;

detecting hybridization between the amplified hALB nucleic acid molecules and the hALB probe via a reference signal from at least one label attached to the hALB probe; and

quantitating transgene copy number by comparison of the target signal relative to the reference signal.

In a further aspect, the present invention provides methods of generating a CAR T cell, comprising:
introducing a CAR transgene into a T cell to obtain a transgene integrated T cell;
determining CAR transgene integration, comprising:
   amplifying nucleic acids from the transgene integrated T cell with a first CAR primer comprising a nucleic acid sequence selected from the group consisting of SEQ ID NO: 2, SEQ ID NO: 5, SEQ ID NO: 8, SEQ ID NO: 11, SEQ ID NO: 14, SEQ ID NO: 17, SEQ ID NO: 20 and combinations thereof, and a second CAR primer comprising a nucleic acid sequence selected from the group consisting of SEQ ID NO: 3, SEQ ID NO:6, SEQ ID NO: 9, SEQ ID NO: 12, SEQ ID NO: 15, SEQ ID NO: 18, SEQ ID NO: 21 and combinations thereof, thereby generating amplified CAR nucleic acids;
   amplifying the nucleic acids from the transgene integrated T cell with a first reference gene primer and a second reference gene primer, thereby generating amplified reference gene nucleic acids;
   detecting hybridization between the amplified CAR nucleic acids and a CAR probe comprising a nucleotide sequence selected from the group consisting of SEQ ID NO: 1, SEQ ID NO: 4, SEQ ID NO: 7, SEQ ID NO: 10, SEQ ID NO: 13, SEQ ID NO: 16, SEQ ID NO: 19 and combinations thereof, via a target signal from at least one label attached to the CAR probe;
   detecting hybridization between the amplified reference gene nucleic acids and the reference gene probe via a reference signal from at least one label attached to the reference gene probe;
   and
   quantitating transgene copy number by comparison of the target signal relative to the reference signal;
   and
obtaining a CAR T cell comprising at least one copy of the integrated CAR transgene.

In yet a further aspect, the present invention provides methods of generating a CAR T cell, comprising:
introducing a CAR transgene into a T cell to obtain a transgene integrated T cell; determining CAR transgene integration, comprising:
   contacting nucleic acids from the transgene integrated T cell with a first CAR primer comprising a nucleic acid sequence selected from the group consisting of SEQ ID NO: 2, SEQ ID NO: 5, SEQ ID NO: 8, SEQ ID NO: 11,
SEQ ID NO: 14, SEQ ID NO: 17, SEQ ID NO: 20 and combinations thereof, and contacting the nucleic acids from the transgene integrated T cell with a second CAR primer comprising a nucleic acid sequence selected from the group consisting of SEQ ID NO: 3, SEQ ID NO:6, SEQ ID NO: 9, SEQ ID NO: 12,
SEQ ID NO: 15, SEQ ID NO: 18, SEQ ID NO: 21 and combinations thereof; contacting the nucleic acids from the transgene integrated cell with a first
hALB primer comprising a nucleic acid sequence selected from the group consisting of SEQ ID NO: 23, SEQ ID NO: 26, SEQ ID NO: 29 and combinations thereof, and contacting the nucleic acids from the transgene integrated T cell with a second hALB primer comprising a nucleic acid sequence selected from the group consisting of SEQ ID NO: 24, SEQ ID NO: 27, SEQ ID NO: 30 and combinations thereof;
   contacting the nucleic acids from the transgene integrated T cell with a CAR probe, wherein the CAR probe comprises a nucleotide sequence selected from the group consisting of SEQ ID NO: 1, SEQ ID NO: 4, SEQ ID NO: 7, SEQ ID NO: 10, SEQ ID NO: 13, SEQ ID NO: 16, SEQ ID NO: 19 and combinations thereof;
   contacting the nucleic acids from the transgene integrated T cell with a hALB probe, wherein the hALB probe comprises a nucleotide sequence selected from the group consisting of SEQ ID NO: 22, SEQ ID NO: 25, SEQ ID NO: 28 and combinations thereof;
   amplifying CAR nucleic acids with the first CAR primer and second CAR primer, thereby generating amplified CAR nucleic acid molecules;
   amplifying hALB nucleic acids with the first hALB primer and second hALB primer, thereby generating amplified hALB nucleic acid molecules;
   detecting hybridization between the amplified CAR nucleic acid molecules and the CAR probe via a target signal from at least one label attached to the CAR probe;
   detecting hybridization between the amplified hALB nucleic acid molecules and the hALB probe via a reference signal from at least one label attached to the hALB probe; and
   quantitating transgene copy number by comparison of the target signal relative to the reference signal;
and
obtaining a CAR T cell comprising at least one copy of the integrated CAR transgene.

Aspects of the invention also provide CAR T cells generated by the methods described herein.

In certain embodiments, the step of detecting hybridization between the amplified CAR nucleic acid molecules and the CAR probe step comprises detecting a change in target signal from the at least one label attached to the CAR probe during or after hybridization relative to target signal from the at least one label attached to the CAR probe before hybridization.

In certain embodiments, the detecting hybridization among the amplified hALB nucleic acid molecules and the hALB probe step comprises detecting a change in target signal from the at least one label attached to the hALB probe during or after hybridization relative to target signal from the at least one label attached to the hALB probe before hybridization.

In certain embodiments, at least one of the amplifying steps comprises polymerase chain reaction (PCR), for example, real-time PCR, reverse transcriptase-polymerase chain reaction (RT-PCR), real-time reverse transcriptase-polymerase chain reaction (rt RT-PCR), ligase chain reaction, or transcription-mediated amplification (TMA).

In certain embodiments, the nucleic acids which are amplified are amplicons.

In some embodiments, at least one label attached to the CAR probe comprises a fluorophore. In some embodiments, at least one label attached to the hALB probe comprises a fluorophore.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a gel image from the singleplex primers/probe screening assays.

FIG. 2 shows a gel image of multiplex primers/probe assays.

FIG. 3 shows a gel image of Transgene (FP) Set 1 and (RP) Set 2 multiplexed with hALB Set 1.

FIG. 4A-D show amplification curves for Transgene (FP) Set 1 and (RP) Set 2 and hALB Set 1 standard curves.

FIG. 5A-B show Fresh vs Frozen standard curves (Transgene Target).

FIG. 6 shows Circular vs Linear standard curves (Transgene Target).

FIG. 7 shows characterization vs typical transgene qPCR standard curve.

FIG. 8 shows characterization transgene standard linearity plot.

FIG. 9 shows an example qPCR plate layout.

FIG. 10 shows an example controls qualification qPCR plate layout.

FIG. 11 shows an example transgene linearity plot.

FIG. 12 shows an example hALB linearity plot.

FIG. 13 shows the nucleotide sequence of the human serum albumin (hALB) gene, GenBank accession M12523.1.

FIG. 14 disclosed SEQ ID NO: 11.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

### DETAILED DESCRIPTION

A description of example embodiments follows.

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting. As used herein, the indefinite articles “a”, “an” and “the” should be understood to include plural reference unless the context clearly indicates otherwise.

The term “comprise,” or variations such as “comprises” or “comprising,” as used herein may be used to imply the inclusion of a stated element or integer or group of elements or integers, but not the exclusion of any other element or integer or group of elements or integers.

The present invention relates to kits and methods for quantitating transgene integration into chimeric antigen receptor (CAR) T cells. Further, panels of probes and primers are provided for performing polymerase chain reaction (PCR), e.g., quantitative PCR, for quantitating transgene integration into CAR T cells.

In some embodiments, the transgene qPCR methods and kits described by the present invention comprise a multiplexed quantitative polymerase chain reaction (qPCR) assay designed for the quantitation of a BCMA CAR transgene plasmid integrated into a CAR T drug product. Both (1) a BCMA CAR transgene plasmid (Transgene) and (2) a human albumin (hALB) reference gene, are amplified in this qPCR method. The primer and probe set for the transgene targets can amplify the junction between the CD137 and CD3z regions of the plasmid to ensure that only the BCMA CAR transgene plasmid present and integrated into the CAR T drug product is detected.

Chimeric Antigen Receptors

A chimeric antigen receptor (CAR) is an artificially constructed hybrid protein or polypeptide containing the antigen binding domains of an antibody (scFv) linked to T cell signaling domains. As used herein, the terms “T cells,” “T-cells,” and “T lymphocytes” are used interchangeably. Characteristics of CARs can include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigens independent of antigen processing, thus bypassing a major mechanism of tumor evasion. Moreover, when expressed in T-cells, CARs advantageously do not dimerize with endogenous T cell receptor (TCR) alpha and beta chains.

The CARs described herein provide recombinant polypeptide constructs comprising at least an extracellular antigen binding domain, a transmembrane domain and an intracellular signaling domain (also referred to herein as “a cytoplasmic signaling domain”) comprising a functional signaling domain derived from a stimulatory molecule as defined below. T cells expressing a CAR are referred to herein as CAR T cells, CAR T cells or CAR modified T cells, and these terms are used interchangeably herein. The cell can be genetically modified to express an antibody binding domain on its surface stably, conferring novel antigen specificity that is MHC independent.

In some instances, the T cell is genetically modified to stably express a CAR that combines an antigen recognition domain of a specific antibody with an intracellular domain of the CD3-zeta chain or RcγRI protein into a single chimeric protein. In one embodiment, the stimulatory molecule is the zeta chain associated with the T cell receptor complex.

An “intracellular signaling domain,” as the term is used herein, refers to an intracellular portion of a molecule. It is the functional portion of the protein which acts by transmitting information within the cell to regulate cellular activity via defined signaling pathways by generating second messengers or functioning as effectors by responding to such messengers. The intracellular signaling domain generates a signal that promotes an immune effector function of the CAR containing cell, e.g., a CAR T cell. Examples of immune effector function, e.g., in a CART cell, include cytolytic activity and helper activity, including the secretion of cytokines.

In an embodiment, the intracellular signaling domain can comprise a primary intracellular signaling domain. Example primary intracellular signaling domains include those derived from the molecules responsible for primary stimulation, or antigen dependent simulation. In an embodiment, the intracellular signaling domain can comprise a costimulatory intracellular domain. Example costimulatory intracellular signaling domains include those derived from molecules responsible for costimulatory signals, or antigen independent stimulation. For example, in the case of a CAR T, a primary intracellular signaling domain can comprise a cytoplasmic sequence of a T cell receptor, and a costimulatory intracellular signaling domain can comprise a cytoplasmic sequence from a co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3-zeta, FcR gamma, FcR beta, CD3 gamma, CD3 delta, CD3 epsilon, CD5, CD22, CD79a, CD79b, and CD66d DAP10 and DAP12. In a particular embodiment, the signaling sequence is CD3-zeta.

The term “zeta” or alternatively “zeta chain”, “CD3-zeta” or “TCR-zeta” is defined as the protein provided as GenBank Acc. No. BAG36664.1, or the equivalent residues from a non-human species, e.g., murine, rabbit, primate, mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect, the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to, an MHC class 1 molecule, BTLA and a Toll ligand receptor, as well as OX40, CD2, CD27, CD28, CDS, ICAM-1, LFA-1 (CD11a/CD18) and 4-1BB (also referred herein as “CD137”). In a particular embodiment, the costimulatory molecule is 4-1BB (CD137).

A costimulatory intracellular signaling domain can be the intracellular portion of a costimulatory molecule. A costimulatory molecule can be represented in the following protein families: TNF receptor proteins, immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, a ligand that specifically binds with CD83, and the like.

The intracellular signaling domain can comprise the entire (i.e., full length”) intracellular portion, or the entire native intracellular signaling domain, of the molecule from which it is derived, or a functional fragment thereof.

The term “4-1BB” refers to a member of the tumor necrosis factor receptor (TNFR) superfamily with an amino acid sequence provided as GenBank Acc. No. AAA62478.2, or the equivalent residues from a non-human species, e.g., a mammal (mouse, rodent, monkey, ape and the like); and a “4-1BB costimulatory domain” is defined as amino acid residues 214-255 of GenBank accession no. AAA62478.2, or the equivalent residues from a non-human species, e.g., a mammal (mouse, rodent, monkey, ape and the like).

In some embodiments, the cytoplasmic signaling domain further comprises one or more functional signaling domains derived from at least one costimulatory molecule as defined herein. In one embodiment, the costimulatory molecule is chosen from 4-1BB (i.e., CD137), CD27, CD3-zeta and/or CD28. CD28 is a T cell marker important in T cell co-stimulation. CD27 is a member of the tumor necrosis factor receptor superfamily and acts as a co-stimulatory immune checkpoint molecule. 4-1BB transmits a potent costimulatory signal to T cells, promoting differentiation and enhancing long-term survival of T lymphocytes. CD3-zeta associates with TCRs to produce a signal and contains immunoreceptor tyrosine-based activation motifs (ITAMs).

In one embodiment, the CAR comprises an intracellular hinge domain comprising CD8 and an intracellular T cell receptor signaling domain comprising CD28, 4-1BB, CD3-zeta and combinations thereof.

In a particular embodiment, the CAR comprises CD8a transmembrane, CD137, and CD3z coding regions.

The disclosure further provides primers, probes and related kits useful for quantitating variant plasmids integrated into CAR T products, e.g., functional variants, of the CARs, nucleic acids, polypeptides, and proteins described herein. As used herein, the term “Variant” refers to a polypeptide or a polynucleotide that differs from a reference polypeptide or a reference polynucleotide by one or more modifications for example, substitutions, insertions or deletions. The term “functional variant” as used herein refers to a CAR, polypeptide, or protein having substantial or significant sequence identity or similarity to a parent CAR, polypeptide, or protein, which functional variant retains the biological activity of the CAR, polypeptide, or protein for which it is a variant. Functional variants encompass, e.g., those variants of the CAR, polypeptide, or protein described herein (the parent CAR, polypeptide, or protein) that retain the ability to recognize target cells to a similar extent, the same extent, or to a higher extent, as the parent CAR, polypeptide, or protein. In reference to the parent CAR, polypeptide, or protein, the functional variant can, for example, be at least about 30%, about 40%, about 50%, about 60%, about 75%, about 80%, about 85%, about 90%, about 91%, about 92%, about 93%, about 94% about 95%, about 96%, about 97%, about 98%, about 99% or more identical in amino acid sequence to the parent CAR, polypeptide, or protein.

A functional variant can, for example, comprise the amino acid sequence of the parent CAR, polypeptide, or protein with at least one conservative amino acid substitution. In another embodiment, the functional variants can comprise the amino acid sequence of the parent CAR, polypeptide, or protein with at least one non-conservative amino acid substitution. In this case, the non-conservative amino acid substitution may not interfere with or inhibit the biological activity of the functional variant. The non-conservative amino acid substitution may enhance the biological activity of the functional variant such that the biological activity of the functional variant is increased as compared to the parent CAR, polypeptide, or protein.

Amino acid substitutions of the CARs may be conservative amino acid substitutions. Conservative amino acid substitutions are known in the art, and include amino acid substitutions in which one amino acid having certain physical and/or chemical properties is exchanged for another amino acid that has the same or similar chemical or physical properties. For example, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

The CAR, polypeptide, or protein can consist essentially of the specified amino acid sequence or sequences described herein, such that other components e.g., other amino acids, do not materially change the biological activity of the CAR, polypeptide, or protein.

Examples of modified nucleotides that can be used to generate the recombinant nucleic acids utilized to produce the polypeptides utilized in the methods/kits described herein include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, carboxymethylaminomethyl-2-thiouridine, 5-carboxymethylaminomethyluracil, dihydrouracil, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, beta-D-galactosylqueosine, inosine, N⁶-i sopentenyladenine, 1-methylguanine, 1-methylinosine, 2,2-dimethylguanine, 2-methyladenine, 2-methylguanine, 3-methylcytosine, 5-methylcytosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

The nucleic acid of the invention can comprise any isolated or purified nucleotide sequence which encodes any of the CARs, polypeptides, or proteins, or functional portions or functional variants thereof. Alternatively, the nucleotide sequence can comprise a nucleotide sequence which is degenerate to any of the sequences or a combination of degenerate sequences.

Some embodiments of the invention also utilize an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions.

As described herein “high stringency conditions” means that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-12 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for hybridizing to CAR nucleic acids described herein. It is generally appreciated that conditions can be rendered more stringent by the addition of increasing amounts of formamide.

As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole; however, parts of the vectors can be naturally-occurring. The described recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. The non-naturally occurring or altered nucleotides or internucleotide linkages do not hinder the transcription or replication of the vector.

In an embodiment, the recombinant expression vector can be any suitable recombinant expression vector and can be used to transform or transfect any suitable host. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Examples of plant expression vectors include pBI01, pBI01.2, pBI121, pBI101.3, and pBIN19 (Clontech). Examples of animal expression vectors include pEUK-Cl, pMAM, and pMAMneo (Clontech). The recombinant expression vector may be a viral vector, e.g., a retroviral vector, e.g., a gamma retroviral vector.

In an embodiment, the recombinant expression vectors are prepared using standard recombinant DNA techniques described in, for example, Ausubel F M, Brent R, Kingston R E et al. (eds) (1999) Short Protocols in Molecular Biology, 4th edn. New York: Wiley Green M R and Sambrook J. (2012) Molecular cloning: a laboratory manual, 4th edn. Cold Spring Harbor, N.Y. Constructs of expression vectors, which are circular or linear, can be prepared to contain a replication system functional in a prokaryotic or eukaryotic host cell. Replication systems can be derived, e.g., from ColEl, SV40, 2μ plasmid, λ, bovine papilloma virus, and the like.

In certain embodiments, expression vectors utilized by the present disclosure are linearized for preparation of working stocks of plasmid to make standards and controls.

The recombinant expression vector may comprise regulatory sequences, such as transcription and translation initiation and termination codons, which are specific to the type of host (e.g., bacterium, fungus, plant, or animal) into which the vector is to be introduced, as appropriate, and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the described expression vectors include, for instance, neomycin/G418 resistance genes, histidinol x resistance genes, histidinol resistance genes, tetracycline resistance genes, and ampicillin resistance genes.

The recombinant expression vector can comprise a native or normative promoter operably linked to the nucleotide sequence encoding the CAR, polypeptide, or protein (including functional portions and functional variants thereof), or to the nucleotide sequence which is complementary to or which hybridizes to the nucleotide sequence encoding the CAR, polypeptide, or protein. The selection of promoters, e.g., strong, weak, tissue-specific, inducible and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an RSV promoter, an SV40 promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine daminase, purine nucleoside phosphorylase, and nitroreductase.

Included in the scope of the invention are conjugates, e.g., bioconjugates, comprising any of the CARs, polypeptides, or proteins (including any of the functional portions or variants thereof), host cells, nucleic acids, recombinant expression vectors, populations of host cells, or antibodies, or antigen binding portions thereof. Conjugates, as well as methods of synthesizing conjugates in general, are known in the art (See, for instance, Hudecz, F., Methods Mol. Biol. 298: 209-223 (2005) and Kirin et al., Inorg Chem. 44(15): 5405-5415 (2005)).

In a particular embodiment, the recombinant expression vector utilized in embodiments of the invention is a vector comprising various components of the B cell maturation antigen (BCMA) chimeric antigen receptor. The plasmid is an 8,518 base pair (bp) plasmid containing sequences encoding the various components of the BCMA chimeric antigen receptor, as disclosed by SEQ ID NOs: 175-197, 202-205, 218-227, 239, 261-264, and 271-276 in PCT International Patent Application Publ. No. WO2017/025038 A1, the contents of which are incorporated herein by reference in their entirety. In one aspect, plasmid codes for an extracellular antigen-binding domain, a transmembrane domain and an intracellular signaling domain, wherein the extracellular antigen-binding domain binds the BCMA antigen. As used herein, the terms “B cells,” “B-cells,” and “B lymphocytes” are used interchangeably. In one embodiment, the plasmid comprises a nucleic acid sequence of any of SEQ ID NOs: 175-197, 202-205, 218-227, 239, 261-264, and 271-276 from International Patent Application Publ. No. WO2017/025038 A1. In some embodiments, the plasmid comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence of any one of SEQ ID NOs: 175-197, 202-205, 218-227, 239, 261-264, and 271-276 from International Patent Application Publ. No. WO2017/025038 A1.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

In one embodiment, the present disclosure provides an expression vector comprising the nucleic acid sequence of any of SEQ ID NOs: 175-197, 202-205, 218-227, 239, 261-264, and 271-276 from International Patent Application Publ. No. WO2017/025038 A1.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

Methods of Generating CAR T Cells and Quantitating Transgene Integration into a CAR T Cell

In one aspect, the present invention provides methods for quantitating transgene integration into a CAR T cell, comprising:

contacting nucleic acids from the CAR T cell with a first CAR primer comprising a nucleic acid sequence selected from the group consisting of SEQ ID NO: 2, SEQ ID NO: 5, SEQ ID NO: 8, SEQ ID NO: 11, SEQ ID NO: 14, SEQ ID NO: 17, SEQ ID NO: 20 and combinations thereof, and contacting the nucleic acids from the CAR T cell with a second CAR primer comprising a nucleic acid sequence selected from the group consisting of SEQ ID NO: 3, SEQ ID NO:6, SEQ ID NO: 9, SEQ ID NO: 12, SEQ ID NO: 15, SEQ ID NO: 18, SEQ ID NO: 21 and combinations thereof;

contacting the nucleic acids from the CAR T cell with a first hALB primer comprising a nucleic acid sequence selected from the group consisting of SEQ ID NO: 23, SEQ ID NO: 26, SEQ ID NO: 29 and combinations thereof, and contacting the nucleic acids from the CAR T cell with a second hALB primer comprising a nucleic acid sequence selected from the group consisting of SEQ ID NO: 24, SEQ ID NO: 27, SEQ ID NO: 30 and combinations thereof;

contacting the nucleic acids from the CAR T cell with a CAR probe, wherein the CAR probe comprises a nucleotide sequence selected from the group consisting of SEQ ID NO: 1, SEQ ID NO: 4, SEQ ID NO: 7, SEQ ID NO: 10, SEQ ID NO: 13, SEQ ID NO: 16, SEQ ID NO: 19 and combinations thereof;

contacting the nucleic acids from the CAR T cell with a hALB probe, wherein the hALB probe comprises a nucleotide sequence selected from the group consisting of SEQ ID NO: 22, SEQ ID NO: 25, SEQ ID NO: 28 and combinations thereof;

amplifying CAR amplicons with the first CAR primer and second CAR primer, thereby generating amplified CAR nucleic acid molecules;

amplifying hALB amplicons with the first hALB primer and second hALB primer, thereby generating amplified hALB nucleic acid molecules;

detecting hybridization between the amplified CAR nucleic acid molecules and the CAR probe via a target signal from at least one label attached to the CAR probe;

detecting hybridization between the amplified hALB nucleic acid molecules and the hALB probe via a reference signal from at least one label attached to the hALB probe; and

quantitating transgene copy number by comparison of the target signal relative to the reference signal.

In some embodiments, the contacting steps for the CAR primers are performed in a separate reaction from the hALB primers. In other embodiments, the contacting steps are performed in the same reaction (i.e., multiplexed).

In some embodiments, the contacting steps for the CAR probes are performed in a separate reaction from the hALB probes. In other embodiments, the contacting steps are performed in the same reaction (i.e., multiplexed).

In some embodiments, the amplifying steps for the CAR amplicons are performed in a separate reaction from the hALB amplicons. In other embodiments, the amplifying steps are performed in the same reaction (i.e., multiplexed).

In some embodiments, the detecting steps for the hybridization of CAR nucleic acids and CAR probes are performed in a separate reaction from the hALB nucleic acids and hALB probes. In other embodiments, the detecting steps are performed in the same reaction (i.e., multiplexed).

In some embodiments, the methods involve amplifying CAR nucleic acids with a first CAR primer between about 20 and about 40 nucleotides in length. In some embodiments, the first CAR primer is capable of hybridizing under conditions of high stringency to a CAR nucleic acid sequence set forth as any of SEQ ID NOs: 175-197, 202-205, 218-227, 239, 261-264, and 271-276 from International Patent Application Publ. No. WO2017/025038 A1.

The primer, i.e., nucleotide sequence, which hybridizes under stringent conditions may hybridize under high stringency conditions.

In some embodiments, the first CAR primer includes a nucleic acid sequence that is at least about 80% identical, at least about 85% identical, at least about 90% identical, or at least about 95% identical to a nucleic acid sequence selected from the group consisting of SEQ ID NO: 2, SEQ ID NO: 5, SEQ ID NO: 8, SEQ ID NO: 11, SEQ ID NO: 14, SEQ ID NO: 17 and SEQ ID NO: 20. In some embodiments, the first CAR primer includes a nucleic acid sequence that is at least about 96% identical, at least about 97% identical, at least about 98% identical, or at least about 99% identical to a nucleic acid sequence selected from the group consisting of SEQ ID NO: 2, SEQ ID NO: 5, SEQ ID NO: 8, SEQ ID NO: 11, SEQ ID NO: 14, SEQ ID NO: 17 and SEQ ID NO: 20. In specific embodiments, the first CAR primer includes a nucleic acid sequence that is at least about 95% identical to a nucleic acid sequence selected from the group consisting of SEQ ID NO: 2, SEQ ID NO: 5, SEQ ID NO: 8, SEQ ID NO: 11, SEQ ID NO: 14, SEQ ID NO: 17 and SEQ ID NO: 20.

In some embodiments, the first CAR primer includes a nucleic acid sequence that is at least about 80% identical, at least about 85% identical, at least about 90% identical, or at least about 95% identical to a nucleic acid sequence set forth as SEQ ID NO: 2. In some embodiments, the first CAR primer includes a nucleic acid sequence that is at least about 96% identical, at least about 97% identical, at least about 98% identical, or at least about 99% identical to a nucleic acid sequence set forth as SEQ ID NO: 2. In specific embodiments, the first CAR primer includes a nucleic acid sequence that is at least about 95% identical to a nucleic acid sequence set forth as SEQ ID NO: 2.

In some embodiments, the first CAR primer includes a nucleic acid sequence that is at least about 80% identical, at least about 85% identical, at least about 90% identical, or at least about 95% identical to a nucleic acid sequence set forth as SEQ ID NO: 5. In some embodiments, the first CAR primer includes a nucleic acid sequence that is at least about 96% identical, at least about 97% identical, at least about 98% identical, or at least about 99% identical to a nucleic acid sequence set forth as SEQ ID NO: 5. In specific embodiments, the first CAR primer includes a nucleic acid sequence that is at least about 95% identical to a nucleic acid sequence set forth as SEQ ID NO: 5.

In some embodiments, the first CAR primer includes a nucleic acid sequence that is at least about 80% identical, at least about 85% identical, at least about 90% identical, or at least about 95% identical to a nucleic acid sequence set forth as SEQ ID NO: 8. In some embodiments, the first CAR primer includes a nucleic acid sequence that is at least about 96% identical, at least about 97% identical, at least about 98% identical, or at least about 99% identical to a nucleic acid sequence set forth as SEQ ID NO: 8. In specific embodiments, the first CAR primer includes a nucleic acid sequence that is at least about 95% identical to a nucleic acid sequence set forth as SEQ ID NO: 8.

In some embodiments, the first CAR primer includes a nucleic acid sequence that is at least about 80% identical, at least about 85% identical, at least about 90% identical, or at least about 95% identical to a nucleic acid sequence set forth as SEQ ID NO: 11.

In some embodiments, the first CAR primer includes a nucleic acid sequence that is at least about 96% identical, at least about 97% identical, at least about 98% identical, or at least about 99% identical to a nucleic acid sequence set forth as SEQ ID NO: 11. In specific embodiments, the first CAR primer includes a nucleic acid sequence that is at least about 95% identical to a nucleic acid sequence set forth as SEQ ID NO: 11.

In some embodiments, the first CAR primer includes a nucleic acid sequence that is at least about 80% identical, at least about 85% identical, at least about 90% identical, or at least about 95% identical to a nucleic acid sequence set forth as SEQ ID NO: 14. In some embodiments, the first CAR primer includes a nucleic acid sequence that is at least about 96% identical, at least about 97% identical, at least about 98% identical, or at least about 99% identical to a nucleic acid sequence set forth as SEQ ID NO: 14. In specific embodiments, the first CAR primer includes a nucleic acid sequence that is at least about 95% identical to a nucleic acid sequence set forth as SEQ ID NO: 14.

In some embodiments, the first CAR primer includes a nucleic acid sequence that is at least about 80% identical, at least about 85% identical, at least about 90% identical, or at least about 95% identical to a nucleic acid sequence set forth as SEQ ID NO: 17. In some embodiments, the first CAR primer includes a nucleic acid sequence that is at least about 96% identical, at least about 97% identical, at least about 98% identical, or at least about 99% identical to a nucleic acid sequence set forth as SEQ ID NO: 17. In specific embodiments, the first CAR primer includes a nucleic acid sequence that is at least about 95% identical to a nucleic acid sequence set forth as SEQ ID NO: 17.

In some embodiments, the first CAR primer includes a nucleic acid sequence that is at least about 80% identical, at least about 85% identical, at least about 90% identical, or at least about 95% identical to a nucleic acid sequence set forth as SEQ ID NO: 20. In some embodiments, the first CAR primer includes a nucleic acid sequence that is at least about 96% identical, at least about 97% identical, at least about 98% identical, or at least about 99% identical to a nucleic acid sequence set forth as SEQ ID NO: 20. In specific embodiments, the first CAR primer includes a nucleic acid sequence that is at least about 95% identical to a nucleic acid sequence set forth as SEQ ID NO: 20.

In some embodiments, the methods involve amplifying CAR nucleic acids with a second CAR primer between about 20 and about 40 nucleotides in length. In some embodiments, the second CAR primer is capable of hybridizing under conditions of high stringency to a CAR nucleic acid sequence set forth as any of SEQ ID NOs: 175-197, 202-205, 218-227, 239, 261-264, and 271-276 from PCT International Patent Application Publ. No. WO2017/025038 A1.

In some embodiments, the second CAR primer includes a nucleic acid sequence that is at least about 80% identical, at least about 85% identical, at least about 90% identical, or at least about 95% identical to a nucleic acid sequence selected from the group consisting of SEQ ID NO: 3, SEQ ID NO:6, SEQ ID NO: 9, SEQ ID NO: 12, SEQ ID NO: 15, SEQ ID NO: 18 and SEQ ID NO: 21. In some embodiments, the second CAR primer includes a nucleic acid sequence that is at least about 96% identical, at least about 97% identical, at least about 98% identical, or at least about 99% identical to a nucleic acid sequence selected from the group consisting of SEQ ID NO: 3, SEQ ID NO:6, SEQ ID NO: 9, SEQ ID NO: 12, SEQ ID NO: 15, SEQ ID NO: 18 and SEQ ID NO: 21. In specific embodiments, the second CAR primer includes a nucleic acid sequence that is at least about 95% identical to a nucleic acid sequence selected from the group consisting of SEQ ID NO: 3, SEQ ID NO:6, SEQ ID NO: 9, SEQ ID NO: 12, SEQ ID NO: 15, SEQ ID NO: 18 and SEQ ID NO: 21.

In some embodiments, the second CAR primer includes a nucleic acid sequence that is at least about 80% identical, at least about 85% identical, at least about 90% identical, or at least about 95% identical to a nucleic acid sequence set forth as SEQ ID NO: 3. In some embodiments, the second CAR primer includes a nucleic acid sequence that is at least about 96% identical, at least about 97% identical, at least about 98% identical, or at least about 99% identical to a nucleic acid sequence set forth as SEQ ID NO: 3. In specific embodiments, the second CAR primer includes a nucleic acid sequence that is at least about 95% identical to a nucleic acid sequence set forth as SEQ ID NO: 3.

In some embodiments, the second CAR primer includes a nucleic acid sequence that is at least about 80% identical, at least about 85% identical, at least about 90% identical, or at least about 95% identical to a nucleic acid sequence set forth as SEQ ID NO: 6. In some embodiments, the second CAR primer includes a nucleic acid sequence that is at least about 96% identical, at least about 97% identical, at least about 98% identical, or at least about 99% identical to a nucleic acid sequence set forth as SEQ ID NO: 6. In specific embodiments, the second CAR primer includes a nucleic acid sequence that is at least about 95% identical to a nucleic acid sequence set forth as SEQ ID NO: 6.

In some embodiments, the second CAR primer includes a nucleic acid sequence that is at least about 80% identical, at least about 85% identical, at least about 90% identical, or at least about 95% identical to a nucleic acid sequence set forth as SEQ ID NO: 9. In some embodiments, the second CAR primer includes a nucleic acid sequence that is at least about 96% identical, at least about 97% identical, at least about 98% identical, or at least about 99% identical to a nucleic acid sequence set forth as SEQ ID NO: 9. In specific embodiments, the second CAR primer includes a nucleic acid sequence that is at least about 95% identical to a nucleic acid sequence set forth as SEQ ID NO: 9.

In some embodiments, the second CAR primer includes a nucleic acid sequence that is at least about 80% identical, at least about 85% identical, at least about 90% identical, or at least about 95% identical to a nucleic acid sequence set forth as SEQ ID NO: 12. In some embodiments, the second CAR primer includes a nucleic acid sequence that is at least about 96% identical, at least about 97% identical, at least about 98% identical, or at least about 99% identical to a nucleic acid sequence set forth as SEQ ID NO: 12. In specific embodiments, the second CAR primer includes a nucleic acid sequence that is at least about 95% identical to a nucleic acid sequence set forth as SEQ ID NO: 12.

In some embodiments, the second CAR primer includes a nucleic acid sequence that is at least about 80% identical, at least about 85% identical, at least about 90% identical, or at least about 95% identical to a nucleic acid sequence set forth as SEQ ID NO: 15. In some embodiments, the second CAR primer includes a nucleic acid sequence that is at least about 96% identical, at least about 97% identical, at least about 98% identical, or at least about 99% identical to a nucleic acid sequence set forth as SEQ ID NO: 15. In specific embodiments, the second CAR primer includes a nucleic acid sequence that is at least about 95% identical to a nucleic acid sequence set forth as SEQ ID NO: 15.

In some embodiments, the second CAR primer includes a nucleic acid sequence that is at least about 80% identical, at least about 85% identical, at least about 90% identical, or at least about 95% identical to a nucleic acid sequence set forth as SEQ ID NO: 18. In some embodiments, the second CAR primer includes a nucleic acid sequence that is at least about 96% identical, at least about 97% identical, at least about 98% identical, or at least about 99% identical to a nucleic acid sequence set forth as SEQ ID NO: 18. In specific embodiments, the second CAR primer includes a nucleic acid sequence that is at least about 95% identical to a nucleic acid sequence set forth as SEQ ID NO: 18.

In some embodiments, the second CAR primer includes a nucleic acid sequence that is at least about 80% identical, at least about 85% identical, at least about 90% identical, or at least about 95% identical to a nucleic acid sequence set forth as SEQ ID NO: 21. In some embodiments, the second CAR primer includes a nucleic acid sequence that is at least about 96% identical, at least about 97% identical, at least about 98% identical, or at least about 99% identical to a nucleic acid sequence set forth as SEQ ID NO: 21. In specific embodiments, the second CAR primer includes a nucleic acid sequence that is at least about 95% identical to a nucleic acid sequence set forth as SEQ ID NO: 21.

In some embodiments, the methods involve hybridizing a CAR nucleic acid molecule to a CAR specific probe between about 20 and about 40 nucleotides in length, and detecting hybridization between the CAR nucleic acid and the probe. In some embodiments, the probe is detectably labeled. In some embodiments, the CAR specific probe is capable of hybridizing under conditions of high stringency to CAR nucleic acid sequence set forth as any of SEQ ID NOs: 175-197, 202-205, 218-227, 239, 261-264, and 271-276 from PCT International Patent Application Publ. No. WO2017/025038 A1.

In some embodiments, the detecting hybridization steps can be performed by traditional molecular biology techniques known in the art, such as, but not limited to, gel electrophoresis, Southern blot, and/or the like.

In some embodiments, the CAR specific probe includes a nucleic acid sequence that is at least about 80% identical, at least about 85% identical, at least about 90% identical, or at least about 95% identical to a nucleic acid sequence selected from the group consisting of SEQ ID NO: 1, SEQ ID NO: 4, SEQ ID NO: 7, SEQ ID NO: 10, SEQ ID NO: 13, SEQ ID NO: 16 and SEQ ID NO: 19.

In some embodiments, the probe includes a nucleic acid sequence that is at least about 96% identical, at least about 97% identical, at least about 98% identical, or at least about 99% identical to a nucleic acid sequence selected from the group consisting of SEQ ID NO: 1, SEQ ID NO: 4, SEQ ID NO: 7, SEQ ID NO: 10, SEQ ID NO: 13, SEQ ID NO: 16 and SEQ ID NO: 19. In specific embodiments, the probe includes a nucleic acid sequence that is at least about 95% identical to a nucleic acid sequence selected from the group consisting of SEQ ID NO: 1, SEQ ID NO: 4, SEQ ID NO: 7, SEQ ID NO: 10, SEQ ID NO: 13, SEQ ID NO: 16 and SEQ ID NO: 19.

In some embodiments, the CAR specific probe includes a nucleic acid sequence that is at least about 80% identical, at least about 85% identical, at least about 90% identical, or at least about 95% identical to a nucleic acid sequence set forth as SEQ ID NO: 1. In some embodiments, the probe includes a nucleic acid sequence that is at least about 96% identical, at least about 97% identical, at least about 98% identical, or at least about 99% identical to a nucleic acid sequence set forth as SEQ ID NO: 1. In specific embodiments, the probe includes a nucleic acid sequence that is at least about 95% identical to a nucleic acid sequence set forth as SEQ ID NO: 1.

In some embodiments, the CAR specific probe includes a nucleic acid sequence that is at least about 80% identical, at least about 85% identical, at least about 90% identical, or at least about 95% identical to a nucleic acid sequence set forth as SEQ ID NO: 4. In some embodiments, the probe includes a nucleic acid sequence that is at least about 96% identical, at least about 97% identical, at least about 98% identical, or at least about 99% identical to a nucleic acid sequence set forth as SEQ ID NO: 4. In specific embodiments, the probe includes a nucleic acid sequence that is at least about 95% identical to a nucleic acid sequence set forth as SEQ ID NO: 4.

In some embodiments, the CAR specific probe includes a nucleic acid sequence that is at least about 80% identical, at least about 85% identical, at least about 90% identical, or at least about 95% identical to a nucleic acid sequence set forth as SEQ ID NO: 7. In some embodiments, the probe includes a nucleic acid sequence that is at least about 96% identical, at least about 97% identical, at least about 98% identical, or at least about 99% identical to a nucleic acid sequence set forth as SEQ ID NO: 7. In specific embodiments, the probe includes a nucleic acid sequence that is at least about 95% identical to a nucleic acid sequence set forth as SEQ ID NO: 7.

In some embodiments, the CAR specific probe includes a nucleic acid sequence that is at least about 80% identical, at least about 85% identical, at least about 90% identical, or at least about 95% identical to a nucleic acid sequence set forth as SEQ ID NO: 10. In some embodiments, the probe includes a nucleic acid sequence that is at least about 96% identical, at least about 97% identical, at least about 98% identical, or at least about 99% identical to a nucleic acid sequence set forth as SEQ ID NO: 10. In specific embodiments, the probe includes a nucleic acid sequence that is at least about 95% identical to a nucleic acid sequence set forth as SEQ ID NO: 10.

In some embodiments, the CAR specific probe includes a nucleic acid sequence that is at least about 80% identical, at least about 85% identical, at least about 90% identical, or at least about 95% identical to a nucleic acid sequence set forth as SEQ ID NO: 13. In some embodiments, the probe includes a nucleic acid sequence that is at least about 96% identical, at least about 97% identical, at least about 98% identical, or at least about 99% identical to a nucleic acid sequence set forth as SEQ ID NO: 13. In specific embodiments, the probe includes a nucleic acid sequence that is at least about 95% identical to a nucleic acid sequence set forth as SEQ ID NO: 13.

In some embodiments, the CAR specific probe includes a nucleic acid sequence that is at least about 80% identical, at least about 85% identical, at least about 90% identical, or at least about 95% identical to a nucleic acid sequence set forth as SEQ ID NO: 16. In some embodiments, the probe includes a nucleic acid sequence that is at least about 96% identical, at least about 97% identical, at least about 98% identical, or at least about 99% identical to a nucleic acid sequence set forth as SEQ ID NO: 16. In specific embodiments, the probe includes a nucleic acid sequence that is at least about 95% identical to a nucleic acid sequence set forth as SEQ ID NO: 16.

In some embodiments, the CAR specific probe includes a nucleic acid sequence that is at least about 80% identical, at least about 85% identical, at least about 90% identical, or at least about 95% identical to a nucleic acid sequence set forth as SEQ ID NO: 19. In some embodiments, the probe includes a nucleic acid sequence that is at least about 96% identical, at least about 97% identical, at least about 98% identical, or at least about 99% identical to a nucleic acid sequence set forth as SEQ ID NO: 19. In specific embodiments, the probe includes a nucleic acid sequence that is at least about 95% identical to a nucleic acid sequence set forth as SEQ ID NO: 19.

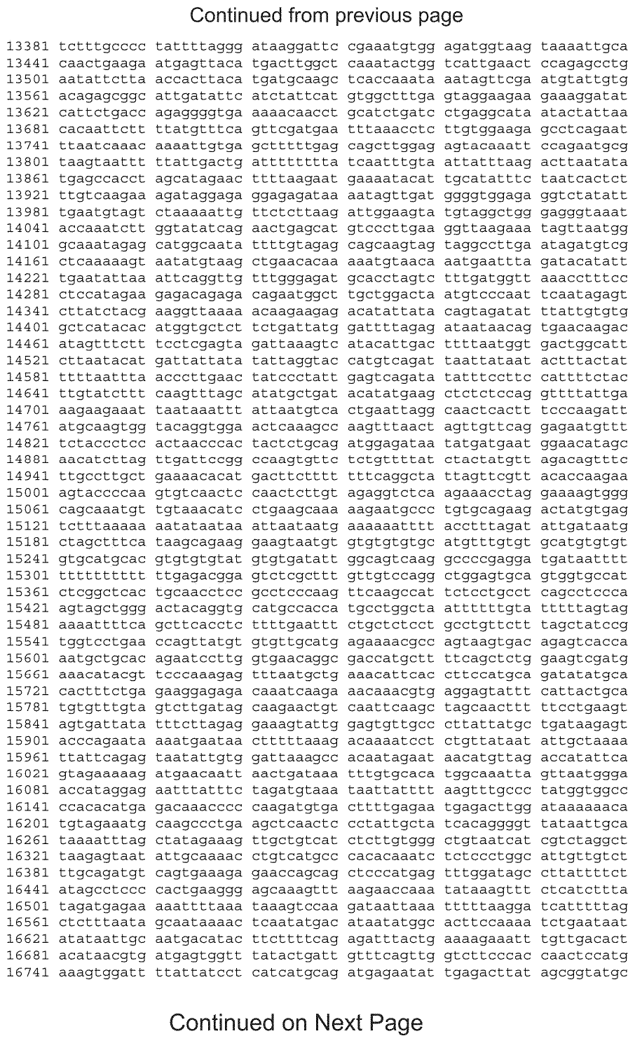

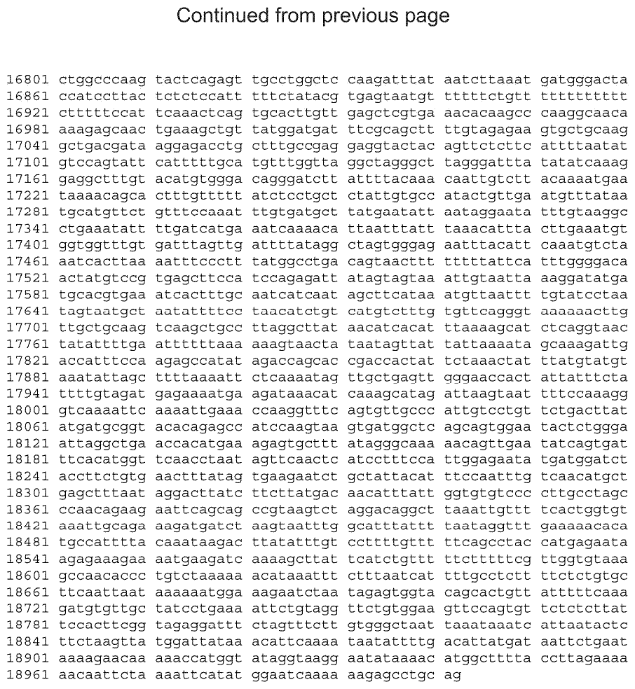

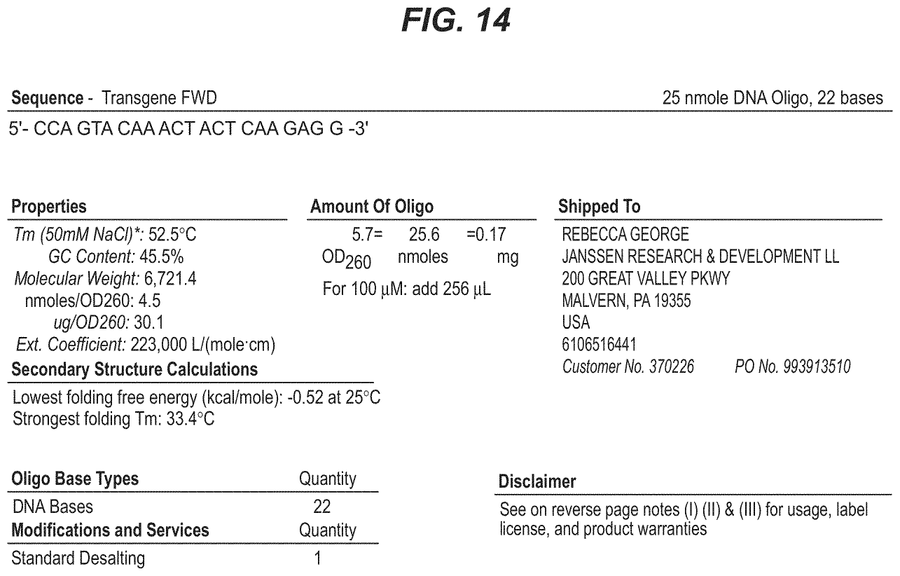

In some embodiments, the methods involve amplifying hALB nucleic acids with a first hALB primer between about 20 and about 40 nucleotides in length. In some embodiments, the first hALB primer is capable of hybridizing under conditions of high stringency to a hALB nucleic acid sequence set forth as SEQ ID NO:31 (FIG. 13).

In some embodiments, the first hALB primer includes a nucleic acid sequence that is at least about 80% identical, at least about 85% identical, at least about 90% identical, or at least about 95% identical to a nucleic acid sequence selected from the group consisting of SEQ ID NO: 23, SEQ ID NO: 26 and SEQ ID NO: 29. In some embodiments, the first hALB primer includes a nucleic acid sequence that is at least about 96% identical, at least about 97% identical, at least about 98% identical, or at least about 99% identical to a nucleic acid sequence selected from the group consisting of SEQ ID NO: 23, SEQ ID NO: 26 and SEQ ID NO: 29. In specific embodiments, the first hALB primer includes a nucleic acid sequence that is at least about 95% identical to a nucleic acid sequence selected from the group consisting of SEQ ID NO: 23, SEQ ID NO: 26 and SEQ ID NO: 29.

In some embodiments, the first hALB primer includes a nucleic acid sequence that is at least about 80% identical, at least about 85% identical, at least about 90% identical, or at least about 95% identical to a nucleic acid sequence set forth as SEQ ID NO: 23. In some embodiments, the first hALB primer includes a nucleic acid sequence that is at least about 96% identical, at least about 97% identical, at least about 98% identical, or at least about 99% identical to a nucleic acid sequence set forth as SEQ ID NO: 23. In specific embodiments, the first hALB primer includes a nucleic acid sequence that is at least about 95% identical to a nucleic acid sequence set forth as SEQ ID NO: 23.

In some embodiments, the first hALB primer includes a nucleic acid sequence that is at least about 80% identical, at least about 85% identical, at least about 90% identical, or at least about 95% identical to a nucleic acid sequence set forth as SEQ ID NO: 26. In some embodiments, the first hALB primer includes a nucleic acid sequence that is at least about 96% identical, at least about 97% identical, at least about 98% identical, or at least about 99% identical to a nucleic acid sequence set forth as SEQ ID NO: 26. In specific embodiments, the first hALB primer includes a nucleic acid sequence that is at least about 95% identical to a nucleic acid sequence set forth as SEQ ID NO: 26.

In some embodiments, the first hALB primer includes a nucleic acid sequence that is at least about 80% identical, at least about 85% identical, at least about 90% identical, or at least about 95% identical to a nucleic acid sequence set forth as SEQ ID NO: 29. In some embodiments, the first hALB primer includes a nucleic acid sequence that is at least about 96% identical, at least about 97% identical, at least about 98% identical, or at least about 99% identical to a nucleic acid sequence set forth as SEQ ID NO: 29. In specific embodiments, the first hALB primer includes a nucleic acid sequence that is at least about 95% identical to a nucleic acid sequence set forth as SEQ ID NO: 29.

In some embodiments, the methods involve amplifying hALB nucleic acids with a second hALB primer between about 20 and about 40 nucleotides in length. In some embodiments, the second hALB primer is capable of hybridizing under conditions of high stringency to a hALB nucleic acid sequence set forth as SEQ ID NO:31.

In some embodiments, the second hALB primer includes a nucleic acid sequence that is at least about 80% identical, at least about 85% identical, at least about 90% identical, or at least about 95% identical to a nucleic acid sequence selected from the group consisting of SEQ ID NO: 24, SEQ ID NO: 27 and SEQ ID NO: 30. In some embodiments, the second hALB primer includes a nucleic acid sequence that is at least about 96% identical, at least about 97% identical, at least about 98% identical, or at least about 99% identical to a nucleic acid sequence selected from the group consisting of SEQ ID NO: 24, SEQ ID NO: 27 and SEQ ID NO: 30. In specific embodiments, the second hALB primer includes a nucleic acid sequence that is at least about 95% identical to a nucleic acid sequence selected from the group consisting of SEQ ID NO: 24, SEQ ID NO: 27 and SEQ ID NO: 30.

In some embodiments, the second hALB primer includes a nucleic acid sequence that is at least about 80% identical, at least about 85% identical, at least about 90% identical, or at least about 95% identical to a nucleic acid sequence set forth as SEQ ID NO: 24. In some embodiments, the second hALB primer includes a nucleic acid sequence that is at least about 96% identical, at least about 97% identical, at least about 98% identical, or at least about 99% identical to a nucleic acid sequence set forth as SEQ ID NO: 24. In specific embodiments, the second hALB primer includes a nucleic acid sequence that is at least about 95% identical to a nucleic acid sequence set forth as SEQ ID NO: 24.

In some embodiments, the second hALB primer includes a nucleic acid sequence that is at least about 80% identical, at least about 85% identical, at least about 90% identical, or at least about 95% identical to a nucleic acid sequence set forth as SEQ ID NO: 27. In some embodiments, the second hALB primer includes a nucleic acid sequence that is at least about 96% identical, at least about 97% identical, at least about 98% identical, or at least about 99% identical to a nucleic acid sequence set forth as SEQ ID NO: 27. In specific embodiments, the second hALB primer includes a nucleic acid sequence that is at least about 95% identical to a nucleic acid sequence set forth as SEQ ID NO: 27.

In some embodiments, the second hALB primer includes a nucleic acid sequence that is at least about 80% identical, at least about 85% identical, at least about 90% identical, or at least about 95% identical to a nucleic acid sequence set forth as SEQ ID NO: 30. In some embodiments, the second hALB primer includes a nucleic acid sequence that is at least about 96% identical, at least about 97% identical, at least about 98% identical, or at least about 99% identical to a nucleic acid sequence set forth as SEQ ID NO: 30. In specific embodiments, the second hALB primer includes a nucleic acid sequence that is at least about 95% identical to a nucleic acid sequence set forth as SEQ ID NO: 30.

In some embodiments, the methods involve hybridizing a hALB nucleic acid molecule to a hALB specific probe between about 20 and about 40 nucleotides in length, and detecting hybridization between the hALB nucleic acid and the probe. In some embodiments, the probe is detectably labeled. In some embodiments, the hALB specific probe is capable of hybridizing under conditions of high stringency to hALB nucleic acid sequence set forth as SEQ ID NO:31.

In some embodiments, the detecting hybridization steps can be performed by traditional molecular biology techniques known in the art, such as, but not limited to, gel electrophoresis, Southern blot, and/or the like.

In some embodiments, the probe includes a nucleic acid sequence that is at least about 80% identical, at least about 85% identical, at least about 90% identical, or at least about 95% identical to a nucleic acid sequence selected from the group consisting of SEQ ID NO: 22, SEQ ID NO: 25 and SEQ ID NO: 28. In some embodiments, the probe includes a nucleic acid sequence that is at least about 96% identical, at least about 97% identical, at least about 98% identical, or at least about 99% identical to a nucleic acid sequence selected from the group consisting of SEQ ID NO: 22, SEQ ID NO: 25 and SEQ ID NO: 28. In specific embodiments, the probe includes a nucleic acid sequence that is at least about 95% identical to a nucleic acid sequence selected from the group consisting of SEQ ID NO: 22, SEQ ID NO: 25 and SEQ ID NO: 28.

In some embodiments, the probe includes a nucleic acid sequence that is at least about 80% identical, at least about 85% identical, at least about 90% identical, or at least about 95% identical to a nucleic acid sequence set forth as SEQ ID NO: 22. In some embodiments, the probe includes a nucleic acid sequence that is at least about 96% identical, at least about 97% identical, at least about 98% identical, or at least about 99% identical to a nucleic acid sequence set forth as SEQ ID NO: 22. In specific embodiments, the probe includes a nucleic acid sequence that is at least about 95% identical to a nucleic acid sequence set forth as SEQ ID NO: 22.

In some embodiments, the probe includes a nucleic acid sequence that is at least about 80% identical, at least about 85% identical, at least about 90% identical, or at least about 95% identical to a nucleic acid sequence set forth as SEQ ID NO: 25. In some embodiments, the probe includes a nucleic acid sequence that is at least about 96% identical, at least about 97% identical, at least about 98% identical, or at least about 99% identical to a nucleic acid sequence set forth as SEQ ID NO: 25. In specific embodiments, the probe includes a nucleic acid sequence that is at least about 95% identical to a nucleic acid sequence set forth as SEQ ID NO: 25.

In some embodiments, the probe includes a nucleic acid sequence that is at least about 80% identical, at least about 85% identical, at least about 90% identical, or at least about 95% identical to a nucleic acid sequence set forth as SEQ ID NO: 28. In some embodiments, the probe includes a nucleic acid sequence that is at least about 96% identical, at least about 97% identical, at least about 98% identical, or at least about 99% identical to a nucleic acid sequence set forth as SEQ ID NO: 28. In specific embodiments, the probe includes a nucleic acid sequence that is at least about 95% identical to a nucleic acid sequence set forth as SEQ ID NO: 28.

In a specific aspect, the present invention provides methods for quantitating transgene integration into a CAR T cell, comprising:

contacting nucleic acids from the CAR T cell with a first CAR primer comprising a nucleic acid sequence of SEQ ID NO: 11 and contacting the nucleic acids from the CAR T cell with a second CAR primer comprising a nucleic acid sequence of SEQ ID NO: 12;

contacting the nucleic acids from the CAR T cell with a first hALB primer comprising a nucleic acid sequence of SEQ ID NO: 23 and contacting the nucleic acids from the CAR T cell with a second hALB primer comprising a nucleic acid sequence of SEQ ID NO: 24;

contacting the nucleic acids from the CAR T cell with a CAR probe, wherein the CAR probe comprises a nucleotide sequence of SEQ ID NO: 10;

contacting the nucleic acids from the CAR T cell with a hALB probe, wherein the hALB probe comprises a nucleotide sequence of SEQ ID NO: 22;

amplifying CAR nucleic acids with the first CAR primer and second CAR primer, thereby generating amplified CAR nucleic acid molecules;

amplifying hALB nucleic acids with the first hALB primer and second hALB primer, thereby generating amplified hALB nucleic acid molecules;

detecting hybridization between the amplified CAR nucleic acid molecules and the CAR probe via a target signal from at least one label attached to the CAR probe;

detecting hybridization between the amplified hALB nucleic acid molecules and the hALB probe via a reference signal from at least one label attached to the hALB probe; and quantitating transgene copy number by comparison of the target signal relative to the reference signal.

In certain embodiments, the detecting hybridization among the amplified CAR nucleic acid molecules and the CAR probe step comprises detecting a change in target signal from the at least one label attached to the CAR probe during or after hybridization relative to target signal from the at least one label attached to the CAR probe before hybridization.

In certain embodiments, the detecting hybridization among the amplified hALB nucleic acid molecules and the hALB probe step comprises detecting a change in target signal from the at least one label attached to the hALB probe during or after hybridization relative to target signal from the at least one label attached to the hALB probe before hybridization.

In another aspect, the present invention provides methods for quantitating transgene integration into a CAR T cell, comprising:

amplifying nucleic acids from the CAR T cell with a first CAR primer comprising a nucleic acid sequence of SEQ ID NO:11 and a second CAR primer comprising a nucleic acid sequence of SEQ ID NO: 12, thereby generating amplified CAR nucleic acids;

amplifying the nucleic acids from the CAR T cell with a first reference gene primer and a second reference gene primer, thereby generating amplified reference gene nucleic acids;

detecting hybridization between the amplified CAR nucleic acids and a CAR probe comprising a nucleotide sequence of SEQ ID NO: 10 via a target signal from at least one label attached to the CAR probe;

detecting hybridization between the amplified reference gene nucleic acids and the reference gene probe via a reference signal from at least one label attached to the reference gene probe; and

quantitating transgene copy number by comparison of the target signal relative to the reference signal.

In yet another aspect, the present invention provides methods of generating a CAR T cell, comprising:

introducing a CAR transgene into a T cell to obtain a transgene integrated T cell;

determining CAR transgene integration, comprising:
amplifying nucleic acids from the transgene integrated T cell with a first CAR primer comprising a nucleic acid sequence of SEQ ID NO: 11 and a second CAR primer comprising a nucleic acid sequence of SEQ ID NO: 12, thereby generating amplified CAR nucleic acids;
amplifying the nucleic acids from the transgene integrated T cell with a first reference gene primer and a second reference gene primer, thereby generating amplified reference gene nucleic acids;
detecting hybridization between the amplified CAR nucleic acids and a CAR probe comprising a nucleotide sequence of SEQ ID NO: 10 via a target signal from at least one label attached to the CAR probe;
detecting hybridization between the amplified reference gene nucleic acids and the reference gene probe via a reference signal from at least one label attached to the reference gene probe; and
quantitating transgene copy number by comparison of the target signal relative to the reference signal;

and

obtaining a CAR T cell comprising at least one copy of the integrated CAR transgene.

Aspects of the invention also provide CAR T cells generated by the methods described herein.

A “reference gene” refers to an internal reaction control that have sequences different than the target gene. For a gene to be regarded as a reference, it must meet several important criteria (Chervoneva I, Li Y, Schulz S, Croker S, Wilson C, Waldman S A, Hyslop T. Selection of optimal reference genes for normalization in quantitative RT-PCR. BMC Bioinforma. 2010; 11:253. doi: 10.1186/1471-2105-11-253.). The most important is that its expression level should be unaffected by experimental factors. Also, it should show minimal variability in its expression between tissues and physiological states of the organism. It is desirable to pick a reference gene that would show a similar threshold cycle with the gene of interest. The reference gene should demonstrate the variability resulting from imperfections of the technology used and preparatory procedures—this ensures that any variation in the amount of genetic material will relate to the same extent as the object of research and control. Examples of “reference genes” that fulfill the aforementioned criteria are the basic metabolism genes called housekeeping genes, which, by definition, are involved in processes essential for the survival of cells. The housekeeping genes useful as reference genes should also be expressed in a stable and non-regulated constant level (Thellin O, Zorzi W, Lakaye B, De Borman B, Coumans B, Hennen G, Grisar T, Igout A, Heinen E. Housekeeping genes as internal standards: Use and limits. J Biotechnol. 1999; 75:291-295. doi: 10.1016/S0168-1656(99)00163-7). Housekeeping genes that are useful as “reference genes” in the methods, kits and primers/probes of the present invention include, but are not limited to, LDHA, NONO, PGK1, PPIH, C1orf43, CHMP2A, EMC7, GPI, PSMB2, PSMB4, RAB7A, REEPS, SNRPD3, VCP, and VPS29.

In another aspect, the present invention provides methods for quantitating transgene integration into a chimeric antigen receptor (CAR) T cell, comprising:

contacting nucleic acids from the CAR T cell with a first CAR primer, a second CAR primer, a first reference gene primer and a second reference gene primer, wherein the first CAR primer comprises a nucleic acid sequence of SEQ ID NO: 11 and the second CAR primer comprises a nucleic acid sequence of SEQ ID NO: 12;

amplifying the CAR nucleic acids with the first CAR primer and second CAR primer, thereby generating amplified CAR nucleic acids;

amplifying reference gene nucleic acids with the first reference gene primer and the second reference gene primer, thereby generating amplified reference gene nucleic acids;

detecting hybridization between the amplified CAR nucleic acids and a CAR probe comprising a nucleotide sequence of SEQ ID NO: 10 via a target signal from at least one label attached to the CAR probe;

detecting hybridization between the amplified reference gene nucleic acids and the reference gene probe via a reference signal from at least one label attached to the reference gene probe; and

quantitating transgene copy number by comparison of the target signal relative to the reference signal.

In some embodiments, detecting hybridization among the amplified CAR nucleic acids and the CAR probe comprises detecting a change in target signal from the at least one label attached to the CAR probe during or after hybridization relative to a target signal from the label attached to the CAR probe before hybridization. In some embodiments, detecting hybridization among the amplified reference gene nucleic acid molecules and the reference gene probe comprises detecting a change in target signal from the at least one label attached to the reference gene probe during or after hybridization relative to a target signal from the label attached to the reference gene probe before hybridization. In some embodiments, the at least one label attached to the reference gene probe comprises a fluorophore.

In some embodiments, the detecting hybridization steps can be performed using traditional molecular biology techniques known in the art, such as, but not limited to, gel electrophoresis, Southern blot, and/or the like.

In certain embodiments, at least one of the amplifying steps comprises polymerase chain reaction (PCR), for example, real-time PCR, reverse transcriptase-polymerase chain reaction (RT-PCR), real-time reverse transcriptase-polymerase chain reaction (rt RT-PCR), ligase chain reaction (LCR), or transcription-mediated amplification (TMA).

PCR is well-known by those skilled in the art. It is a method widely used in molecular biology to make many copies of a specific DNA segment. Using PCR, a single copy (or more) of a DNA sequence is exponentially amplified to generate thousands to millions more copies of that specific DNA segment. Most PCR methods rely on thermal cycling. Thermal cycling exposes reactants to repeated cycles of heating and cooling to permit different temperature-dependent reactions—DNA melting and enzyme-driven DNA replication. PCR employs two main reagents—primers (short single strand nucleotide fragments known as oligonucleotides that are a complementary sequence to the target DNA region) and a DNA polymerase. In the first step of PCR, the two strands of the DNA double helix are physically separated at a high temperature by DNA melting. In the second step, the temperature is lowered and the primers bind to the complementary sequences of DNA. The two DNA strands then become templates for DNA polymerase to enzymatically assemble a new DNA strand from free nucleotides available in the reaction mixture. As PCR progresses, the DNA generated is itself used as a template for replication such that the original DNA template is exponentially amplified. Typically, PCR applications employ a heat-stable DNA polymerase, such as Taq polymerase, an enzyme originally isolated from the thermophilic bacterium Thermus aquaticus.

Quantitative PCR or Real Time PCR (qPCR), as known by those skilled in the art, allow the estimation of the amount of a given sequence present in a sample—a technique often applied to quantitatively determine levels of gene expression. Quantitative PCR is an established tool for DNA quantification that measures the accumulation of DNA product after each round of PCR amplification. qPCR allows the quantification and detection of a specific DNA sequence in real time since it measures concentration while the synthesis process is taking place.

Reverse transcription polymerase chain reaction (RT-PCR), as known by those skilled in the art, is a laboratory technique combining reverse transcription of RNA into DNA (complementary DNA or cDNA) and amplification of specific DNA targets using PCR. It is generally used to measure the amount of a specific RNA. This is achieved by monitoring the amplification reaction using fluorescence by a technique called real-time PCR or quantitative PCR (qPCR). Combined RT-PCR and qPCR are routinely used for analysis of gene expression and quantification of RNA.

As known by those skilled in the art, ligase chain reaction (LCR) is a method of DNA amplification. The ligase chain reaction (LCR) is an amplification process that differs from PCR in that it involves a thermostable ligase to join two probes or other molecules together which can then be amplified by standard PCR cycling.

Transcription-mediated amplification (TMA), as known by those skilled in the art, is an isothermal, single-tube nucleic acid amplification system utilizing two enzymes, RNA polymerase and reverse transcriptase. In contrast to PCR and LCR, the TMA method involves RNA transcription (via RNA polymerase) and DNA synthesis (via reverse transcriptase) to produce an RNA amplicon (the source or product of amplification) from a target nucleic acid.

In some embodiments, the methods described by the present disclosure utilize other quantitative PCR methods known in the art, such as but not limited to digital PCR (dPCR).

In some embodiments, the at least one label attached to the CAR probe comprises a fluorophore. In some embodiments, the at least one label attached to the hALB probe comprises a fluorophore. The term “fluorophore” as used herein refers to any fluorescent compound or protein that can be used in the quantification and detection of the nucleotide sequences to which the probes hybridize.

This disclosure also relates to primers capable of hybridizing to and amplifying a CAR nucleic acid, e.g., a nucleic acid sequence spanning a CD137/CD3z junction of a CAR construct. The primers described can be utilized in the methods described herein. In some embodiments, these primers are between about 20 and about 40 nucleotides in length and capable of hybridizing under very high stringency conditions to a CAR nucleic acid sequence set forth as any of SEQ ID NOs: 175-197, 202-205, 218-227, 239, 261-264, and 271-276 from

International Patent Application Publ. No. WO2017/025038 A1. In some embodiments, these primers comprise a nucleic acid sequence at least 95% identical to a nucleic acid sequence set forth as SEQ ID NO: 2, SEQ ID NO: 5, SEQ ID NO: 8, SEQ ID NO: 11, SEQ ID NO: 14, SEQ ID NO: 17, or SEQ ID NO: 20. In some embodiments, these primers further comprise a nucleic acid sequence at least 95% identical to a nucleic acid sequence set forth as SEQ ID NO: 3, SEQ ID NO:6, SEQ ID NO: 9, SEQ ID NO: 12, SEQ ID NO: 15, SEQ ID NO: 18 or SEQ ID NO: 21.

This disclosure also relates to probes capable of hybridizing to and discriminating between various CAR nucleic acid sequences, e.g., various nucleic acid sequences spanning a CD137/CD3z junction of a CAR construct. The probes described can be utilized in the methods described herein. In some embodiments, these probes are between about 20 and about 40 nucleotides in length and capable of hybridizing under very high stringency conditions to a CAR nucleic acid sequence set forth as any of SEQ ID NOs: 175-197, 202-205, 218-227, 239, 261-264, and 271-276 from International Patent Application Publ. No. WO2017/025038 A1. In some embodiments, these probes comprise a nucleic acid sequence at least 95% identical to a nucleic acid sequence set forth as SEQ ID NO: 1, SEQ ID NO: 4, SEQ ID NO: 7, SEQ ID NO: 10, SEQ ID NO: 13, SEQ ID NO: 16, or SEQ ID NO: 19.

In one aspect, the invention provides probe and primer sets comprising a CAR probe comprising a nucleotide sequence of SEQ ID NO: 10 and at least one label attached to the probe; a first CAR primer comprising a nucleic acid sequence of SEQ ID NO: 11; and a second CAR primer comprising a nucleic acid sequence of SEQ ID NO: 12. In one embodiment, the probe and primer sets further comprise a hALB probe comprising a nucleotide sequence of SEQ ID NO: 22 and at least one label attached to the probe; a first hALB primer comprising a nucleic acid sequence of SEQ ID NO: 23; and a second hALB primer comprising a nucleic acid sequence of SEQ ID NO: 24.

In some embodiments, the at least one label comprises a radioactive isotope, an enzyme substrate, a chemiluminescent agent, a fluorophore, a fluorescence quencher, an enzyme, a chemical, or a combination thereof. In some embodiments, labels can be made to luminesce through photochemical, chemical, and electrochemical means.

The invention also provides kits for quantitating transgene integration into a CAR T cell. The term “kit” as used herein refers to a combination of reagents and other materials. It is contemplated that the kit may include reagents such as buffering agents, protein stabilizing reagents, signal producing systems (e.g., florescence signal generating systems), antibodies, control proteins, as well as testing containers (e.g., microtiter plates, etc.). It is not intended that the term “kit” be limited to a particular combination of reagents and/or other materials. In one embodiment, the kit further comprises instructions for using the reagents. The kit may be packaged in any suitable manner, typically with the elements in a single container or various containers as necessary along with a sheet of instructions for carrying out the test. In some embodiments, the kits also include a positive control sample. Kits may be produced in a variety of ways known in the art.

In one aspect, the kits for quantitating transgene integration into a CAR T cell, comprise: a probe comprising a nucleotide sequence of SEQ ID NO: 10 and at least one label attached to the probe; a first primer comprising a nucleic acid sequence of SEQ ID NO: 11; and a second primer comprising a nucleic acid sequence of SEQ ID NO: 12. In one embodiment, the kits further comprise a hALB probe comprising a nucleotide sequence of SEQ ID NO: 22 and at least one label attached to the probe; a first hALB primer comprising a nucleic acid sequence of SEQ ID NO: 23; and a second hALB primer comprising a nucleic acid sequence of SEQ ID NO: 24.

In some embodiments of the kits of the invention, the at least one label attached to the probe comprises a radioactive isotope, an enzyme substrate, a chemiluminescent agent, a fluorophore, a fluorescence quencher, an enzyme, a chemical, or a combination thereof.

In one embodiment, the kits of the invention comprise an array that comprises the probe. In some embodiments, the array is a multi-well plate.

In certain embodiments, the at least one label attached to the hALB probe comprises a radioactive isotope, an enzyme substrate, a chemiluminescent agent, a fluorophore, a fluorescence quencher, an enzyme, a chemical, or a combination thereof.

As used in the aspects described by the invention, the term “label” refers to a moiety which is capable of producing a detectable signal, i.e., which can be detected in small quantities by detection means which generate a signal. Examples of suitable such means include spectroscopic or photochemical means, e.g., fluorescence or luminescence, or biochemical, immunochemical, or chemical means such as changes in physical, biochemical, immunochemical or chemical properties on contact with a detector analysis compound or reaction with a polypeptide or polypeptide/enzyme mixture to form a detectable complex. Thus, as used herein the term “label” is intended to include both moieties that may be detected directly, such as radioisotopes or fluorochromes, and reactive moieties that are detected indirectly via a reaction which forms a detectable product, such as enzymes that are reacted with substrate to form a product that may be detected spectrophotometrically. It is noted that the labeling reagent may contain a radioactive label moiety such as a radioisotope. In one embodiment, the hybridization probe herein is nonradioactively labeled to avoid the disadvantages associated with radioactivity analysis.

For use of label detection schemes in the methods and kits described by the invention, nucleotide bases are labeled by covalently attaching a compound such that a fluorescent or chemiluminescent signal is generated following incorporation of a dNTP into the extending

DNA primer/template. Examples of fluorescent compounds for labeling dNTPs include but are not limited to fluorescein, rhodamine, and BODIPY (4,4-difluoro-4-bora-3a,4a-diaza-s-indacene). See “Handbook of Molecular Probes and Fluorescent Chemicals”, available from Molecular Probes, Inc. (Eugene, Oreg.). Examples of chemiluminescence based compounds that may be used include but are not limited to luminol and dioxetanones (See, Gundennan and McCapra, “Chemiluminescence in Organic Chemistry”, Springer-Verlag, Berlin Heidleberg, 1987).

Fluorescently or chemiluminescently labeled dNTPs are added individually to a DNA template system containing template DNA annealed to the primer, DNA polymerase and the appropriate buffer conditions. After the reaction interval, the excess dNTP is removed, and the system is probed to detect whether a fluorescent or chemiluminescent tagged nucleotide has been incorporated into the DNA template. Detection of the incorporated nucleotide can be accomplished using different methods that will depend on the type of tag utilized.

For fluorescently-tagged dNTPs the DNA template system may be illuminated with optical radiation at a wavelength which is strongly absorbed by the tag entity. Fluorescence from the tag is detected using for example a photodetector together with an optical filter which excludes any scattered light at the excitation wavelength.

In a further embodiment utilizing fluorescent detection in the kits and methods described herein, the fluorescent tag is attached to the dNTP by a photocleavable or chemically cleavable linker, and the tag is detached following the extension reaction and removed from the template system into a detection cell where the presence, and the amount, of the tag is determined by optical excitation at a suitable wavelength and detection of fluorescence. In this embodiment, the possibility of fluorescence quenching, due to the presence of multiple fluorescent tags immediately adjacent to one another on a primer strand which has been extended complementary to a single base repeat region in the template, is minimized, and the accuracy with which the repeat number can be determined is optimized. In addition, excitation of fluorescence in a separate chamber minimizes the possibility of photolytic damage to the DNA primer/template system.

In one embodiment, the probe comprises a 5′ 6-FAM™ (fluorescein) label. 6-AM™ is a single isomer derivative of fluorescein. FAM™ is a fluorescent dye attachment for oligonucleotides and is compatible with most fluorescence detection equipment. It becomes protonated and has decreased fluorescence below pH 7; it is typically used in the pH range 7.5-8.5. FAM™ can be attached to 5′ or 3′ end of oligos.

In one embodiment, the probe comprises a 5′HEX™ (hexachlorofluorescein) label. Hexachlorofluorescein is a chemical relative of fluorescein that is utilized for multiplexed assays with FAM™. HEX™ can be added only to the 5′ end of an oligonucleotide.

The present disclosure also contemplates use of any other labels known in the art to be used for labeling of probes as described herein, such as e.g., but not limited to, VIC®, TET™, JOE™ NED™ PET®, ROX™, TAMRA™, TET™, Texas Red®, ATTO™ 532, Cy3, Tye 563, Tye™ 665, TEX 615™, Cy5, ZEN™, Iowa Black® FQ, Iowa Black® RQ, DABYCL and Yakima Yellow™.

In one embodiment, the probe comprises a fluorescence quencher label. The quencher label can be used as a double quencher in the reactions disclosed herein. In one embodiment, the probe comprises a Iowa Black® FQ quencher. Iowa Black® FQ has a broad absorbance spectra ranging from 420 to 620 nm with peak absorbance at 531 nm. This quencher is utilized with fluorescein and other fluorescent dyes that emit in the green to pink spectral range. The present disclosure contemplates use of any fluorescence quencher labels known in the art, such as e.g., but not limited to, ZEN™, Black Hole Quencher® (BHQ-1, BHQ-2, BHQ-3, etc.).

The transgene qPCR method and kits described by the present invention comprise a multiplexed quantitative polymerase chain reaction (qPCR) assay designed for the quantitation of the BCMA CAR transgene plasmid integrated into a CAR T drug product. There are two targets amplified in this qPCR method: (1) a BCMA CAR transgene plasmid (Transgene) and (2) human albumin (hALB) reference gene. The primer and probe set for the Transgene target amplify the junction between the CD137 and CD3z regions of the plasmid to ensure that only the BCMA CAR transgene plasmid present and integrated into the CAR T drug product is detected. The hALB reference gene copy number results are used to calculate the vector copy number (VCN) per cell reportable result for each sample tested in the qPCR reaction.

A description of example embodiments follows

Embodiment 1. A probe and primer set comprising: a probe comprising a nucleotide sequence of SEQ ID NO: 10 and at least one label attached to the probe; a first primer comprising a nucleic acid sequence of SEQ ID NO: 11; and a second primer comprising a nucleic acid sequence of SEQ ID NO: 12.

Embodiment 2. The probe and primer set of Embodiment 1, wherein the at least one label comprises a radioactive isotope, an enzyme substrate, a chemiluminescent agent, a fluorophore, a fluorescence quencher, an enzyme, a chemical, or a combination thereof.

Embodiment 3. A kit for quantitating transgene integration into a chimeric antigen receptor (CAR) T cell, comprising: a probe comprising a nucleotide sequence of SEQ ID NO: 10 and at least one label attached to the probe; a first primer comprising a nucleic acid sequence of SEQ ID NO: 11; and a second primer comprising a nucleic acid sequence of SEQ ID NO: 12.

Embodiment 4. The kit of Embodiment 3, wherein the at least one label attached to the probe comprises a radioactive isotope, an enzyme substrate, a chemiluminescent agent, a fluorophore, a fluorescence quencher, an enzyme, a chemical, or a combination thereof.

Embodiment 5. The kit of Embodiment 3, wherein the kit comprises an array that comprises the probe.

Embodiment 6. The kit of Embodiment 5, wherein the array is a multi-well plate.

Embodiment 7. The kit of Embodiment 3, wherein the kit further comprises a human albumin (hALB) probe comprising a nucleic acid sequence of SEQ ID NO: 22 and at least one label attached to the hALB probe, a first hALB primer comprising a nucleic acid sequence of SEQ ID NO: 23, and a second hALB primer comprising a nucleic acid sequence of SEQ ID NO: 24.

Embodiment 8. The kit of Embodiment 7, wherein the at least one label attached to the hALB probe comprises a radioactive isotope, an enzyme substrate, a chemiluminescent agent, a fluorophore, a fluorescence quencher, an enzyme, a chemical, or a combination thereof.

Embodiment 9. The kit of Embodiment 3, wherein the kit further comprises a reference gene probe and at least one label attached to the reference gene probe, a first reference gene primer, and a second reference gene primer.

Embodiment 10. A method for quantitating transgene integration into a chimeric antigen receptor (CAR) T cell, comprising:

amplifying nucleic acids from the CAR T cell with a first CAR primer comprising a nucleic acid sequence of SEQ ID NO: 11 and a second CAR primer comprising a nucleic acid sequence of SEQ ID NO: 12, thereby generating amplified CAR nucleic acids;

amplifying the nucleic acids from the CAR T cell with a first hALB primer comprising a nucleic acid sequence of SEQ ID NO: 23 and a second hALB primer comprising a nucleic acid sequence of SEQ ID NO: 24, thereby generating amplified hALB nucleic acids;

detecting hybridization between the amplified CAR nucleic acids and a CAR probe comprising a nucleotide sequence of SEQ ID NO: 10 via a target signal from at least one label attached to the CAR probe;

detecting hybridization between the amplified hALB nucleic acids and the hALB probe comprising a nucleotide sequence of SEQ ID NO: 22 via a reference signal from at least one label attached to the hALB probe; and

quantitating transgene copy number by comparison of the target signal relative to the reference signal.

Embodiment 11. A method for quantitating transgene integration into a chimeric antigen receptor (CAR)-T cell, comprising:

contacting nucleic acids from the CAR T cell with a first CAR primer, a second CAR primer, a first hALB primer and a second hALB primer, wherein the first CAR primer comprises a nucleic acid sequence of SEQ ID NO: 11, the second CAR primer comprises a nucleic acid sequence of SEQ ID NO: 12, the first hALB primer comprises a nucleic acid sequence of SEQ ID NO: 23 and the second hALB primer comprises a nucleic acid sequence of SEQ ID NO: 24;

amplifying the CAR nucleic acids with the first CAR primer and second CAR primer, thereby generating amplified CAR nucleic acids;

amplifying hALB nucleic acids with the first hALB primer and second hALB primer, thereby generating amplified hALB nucleic acids;

detecting hybridization between the amplified CAR nucleic acids and a CAR probe comprising a nucleotide sequence of SEQ ID NO: 10 via a target signal from at least one label attached to the CAR probe;

detecting hybridization between the amplified hALB nucleic acids and the hALB probe via a reference signal from at least one label attached to the hALB probe; and

quantitating transgene copy number by comparison of the target signal relative to the reference signal.

Embodiment 12. The method of Embodiment 10 or 11, wherein detecting hybridization among the amplified CAR nucleic acids and the CAR probe comprises detecting a change in target signal from the at least one label attached to the CAR probe during or after hybridization relative to a target signal from the label attached to the CAR probe before hybridization.

Embodiment 13. The method of Embodiment 10 or 11, wherein detecting hybridization among the amplified hALB nucleic acid molecules and the hALB probe comprises detecting a change in target signal from the at least one label attached to the hALB probe during or after hybridization relative to a target signal from the label attached to the hALB probe before hybridization.

Embodiment 14. The method of Embodiment 10 or 11, wherein the amplifying comprises polymerase chain reaction (PCR).

Embodiment 15. The method of Embodiment 14, wherein the PCR is real-time PCR, reverse transcriptase-polymerase chain reaction (RT-PCR), real-time reverse transcriptase-polymerase chain reaction (rt RT-PCR), digital PCR (dPCR), ligase chain reaction, or transcription-mediated amplification (TMA).

Embodiment 16. The method of Embodiment 10, wherein at least one label attached to the CAR probe comprises a fluorophore.

Embodiment 17. The method of Embodiment 10, wherein at least one label attached to the hALB probe comprises a fluorophore.

Embodiment 18. A method for quantitating transgene integration into a chimeric antigen receptor (CAR) T cell, comprising:

amplifying nucleic acids from the CAR T cell with a first CAR primer comprising a nucleic acid sequence of SEQ ID NO: 11 and a second CAR primer comprising a nucleic acid sequence of SEQ ID NO: 12, thereby generating amplified CAR nucleic acids;

amplifying the nucleic acids from the CAR T cell with a first reference gene primer and a second reference gene primer, thereby generating amplified reference gene nucleic acids;

detecting hybridization between the amplified CAR nucleic acids and a CAR probe comprising a nucleotide sequence of SEQ ID NO: 10 via a target signal from at least one label attached to the CAR probe;

detecting hybridization between the amplified reference gene nucleic acids and the reference gene probe via a reference signal from at least one label attached to the reference gene probe; and

quantitating transgene copy number by comparison of the target signal relative to the reference signal.

Embodiment 19. A method for quantitating transgene integration into a chimeric antigen receptor (CAR) T cell, comprising:

contacting nucleic acids from the CAR T cell with a first CAR primer, a second CAR primer, a first reference gene primer and a second reference gene primer, wherein the first CAR primer comprises a nucleic acid sequence of SEQ ID NO: 11, the second CAR primer comprises a nucleic acid sequence of SEQ ID NO: 12;

amplifying the CAR nucleic acids with the first CAR primer and second CAR primer, thereby generating amplified CAR nucleic acids;

amplifying reference gene nucleic acids with the first reference gene primer and second reference gene primer, thereby generating amplified reference gene nucleic acids;

detecting hybridization between the amplified CAR nucleic acids and a CAR probe comprising a nucleotide sequence of SEQ ID NO: 10 via a target signal from at least one label attached to the CAR probe;

detecting hybridization between the amplified reference gene nucleic acids and the reference gene probe via a reference signal from at least one label attached to the reference gene probe; and

quantitating transgene copy number by comparison of the target signal relative to the reference signal.

Embodiment 20. The method of Embodiment 18 or 19, wherein detecting hybridization among the amplified CAR nucleic acids and the CAR probe comprises detecting a change in target signal from the at least one label attached to the CAR probe during or after hybridization relative to a target signal from the label attached to the CAR probe before hybridization.

Embodiment 21. The method of Embodiment 18 or 19, wherein detecting hybridization among the amplified reference gene nucleic acid molecules and the reference gene probe comprises detecting a change in target signal from the at least one label attached to the reference gene probe during or after hybridization relative to a target signal from the label attached to the reference gene probe before hybridization.

Embodiment 22. The method of Embodiment 18 or 19, wherein the amplifying comprises polymerase chain reaction (PCR).

Embodiment 23. The method of Embodiment 22, wherein the PCR is real-time PCR, reverse transcriptase-polymerase chain reaction (RT-PCR), real-time reverse transcriptase-polymerase chain reaction (rt RT-PCR), digital PCR (dPCR), ligase chain reaction, or transcription-mediated amplification (TMA).

Embodiment 24. The method of Embodiment 18, wherein at least one label attached to the CAR probe comprises a fluorophore.

Embodiment 25. The method of Embodiment 18, wherein at least one label attached to the reference gene probe comprises a fluorophore.

Embodiment 26. A method of generating a chimeric antigen receptor (CAR) T cell, comprising:

introducing a CAR transgene into a T cell to obtain a transgene integrated T cell;

determining CAR transgene integration, comprising:
amplifying nucleic acids from the transgene integrated T cell with a first CAR primer comprising a nucleic acid sequence of SEQ ID NO: 11 and a second CAR primer comprising a nucleic acid sequence of SEQ ID NO: 12, thereby generating amplified CAR nucleic acids;
amplifying the nucleic acids from the transgene integrated T cell with a first reference gene primer and a second reference gene primer, thereby generating amplified reference gene nucleic acids;
detecting hybridization between the amplified CAR nucleic acids and a CAR probe comprising a nucleotide sequence of SEQ ID NO: 10 via a target signal from at least one label attached to the CAR probe;
detecting hybridization between the amplified reference gene nucleic acids and the reference gene probe via a reference signal from at least one label attached to the reference gene probe; and
quantitating transgene copy number by comparison of the target signal relative to the reference signal; and

obtaining a CAR T cell comprising at least one copy of the integrated CAR transgene.

Embodiment 27. The method of Embodiment 26, wherein detecting hybridization among the amplified CAR nucleic acids and the CAR probe comprises detecting a change in target signal from the at least one label attached to the CAR probe during or after hybridization relative to a target signal from the label attached to the CAR probe before hybridization.

Embodiment 28. The method of Embodiment 26, wherein detecting hybridization among the amplified reference gene nucleic acid molecules and the reference gene probe

comprises detecting a change in target signal from the at least one label attached to the reference gene probe during or after hybridization relative to a target signal from the label attached to the reference gene probe before hybridization.

Embodiment 29. The method of Embodiment 26, wherein the amplifying comprises polymerase chain reaction (PCR).

Embodiment 30. The method of Embodiment 29, wherein the PCR is real-time PCR, reverse transcriptase-polymerase chain reaction (RT-PCR), real-time reverse transcriptase-polymerase chain reaction (rt RT-PCR), digital PCR (dPCR), ligase chain reaction, or transcription-mediated amplification (TMA).

Embodiment 31. The method of Embodiment 26, wherein at least one label attached to the CAR probe comprises a fluorophore.

Embodiment 32. The method of Embodiment 26, wherein at least one label attached to the reference gene probe comprises a fluorophore.

Embodiment 33. A CAR T cell generated by the method of any of Embodiments 26-32.

The following examples are provided to describe further some of the embodiments disclosed herein. The examples are intended to illustrate, not to limit, the disclosed embodiments.

### EXAMPLE 1

### Transgene qPCR Method Development

Method Overview:

The example transgene qPCR method described is a multiplexed quantitative polymerase chain reaction (qPCR) assay designed for the quantitation of a BCMA CAR transgene plasmid (referred to in the examples as “the pLLV-LICAR2SIN plasmid”) integrated into a CAR T drug product. The following are amplified in this qPCR method: (1) transgene pLLV-LICAR2SIN plasmid (Transgene) (the transgene having a nucleotide sequence comprising any of SEQ ID NOs: 175-197, 202-205, 218-227, 239, 261-264, and 271-276 from International Patent Application Publ. No. WO2017/025038 A1) and (2) human albumin (hALB) reference gene. The primer and probe set for the Transgene target amplify the junction between the CD137 and CD3z regions of the plasmid to ensure that only the pLLV-LICAR2SIN plasmid present and integrated into the CAR T drug product is detected. The hALB reference gene copy number results are used to calculate the vector copy number (VCN) per cell reportable result for each sample tested in the qPCR reaction. The VNC/cell sample results of the transgene qPCR method are reported for safety, purity and identity of CAR T drug product samples.

Design of the Transgene Primers and Probes:

The BCMA CAR transgene plasmid, termed the “pLLV-LICAR2SIN plasmid,” is an 8,518 base pair (bp) plasmid containing sequences coding for the various different components of a B-cell maturation antigen (BCMA) chimeric antigen receptor (CAR). The Transgene target of the qPCR was required to only detect the pLLV-LICAR2SIN plasmid present and integrated into the CAR T drug product and that the region targeted need specifically belong to the BCMA CAR construct. To ensure the specificity of the Transgene qPCR target, primers and probe pairs designed to target at least one junction between at least two regions of the pLLV-LICAR2SIN plasmid coding for the CAR construct were designed. First, suitable regions of the pLLV-LICAR2SIN plasmid had to be identified.

The longest base pair (bp) coding regions that integrate into the genome of the CAR T drug product and are specific to the CAR construct belong to the two variable heavy chain portions of the BCMA CAR construct. These two regions are separated by a short linker sequence. The nucleotide sequence region of the plasmid corresponding to the two variable heavy chain portions and the linker was entered in the Nucleotide Basic Local Alignment Search Tool (BLAST) site of the National Center for Biotechnology Information (NCBI) (https://blast.ncbi.nlm.nih.gov/Blast.cgi) to determine if these regions may be suitable targets for the Transgene qPCR method. However, the BLAST results gave multiple hits for various immunoglobulin variable regions across many different species, including homo sapiens. Therefore, the coding regions for the two variable heavy chain components of the CAR were determined to not be suitable targets for the Transgene qPCR method.

The junction between the CD137 and CD3z regions of the pLLV-LICAR2SIN plasmid is included in the region of the plasmid that is integrated into the genome of the CAR T drug product and are components of the BCMA CAR. The size of the CD3z coding region of the pLLV-LICAR2SIN plasmid is the second longest bp coding region of the CAR segment of the plasmid, making it a more suitable region to target due to the potential for a greater number of potentially suitable primer and probe pairs that may be found from the larger coding region. The CD3z coding region is directly adjacent to the CD137 coding region of the plasmid. On the opposite side of the CD137 coding region are plasmid backbone sequences that are not specific to the BCMA CAR construct. Therefore, the junction between CD137 and CD3z was the only option suitable to target if the larger CD3z coding region was to be included in the primers and probe design.

The PrimerQuest® Tool from Integrated DNA Technologies, Inc. (IDT) (Coralville, Iowa) (https://www.idtdna.com/Primerquest/Home/Index) was used to design suitable primers and probe pairs to test in the qPCR method development. The nucleotide sequence of the pLLV-LICAR2SIN plasmid corresponding to the CD137 and CD3z coding regions was entered in the PrimerQuest Tool. The optimal primer melting temperature (Tm) was set to 60° C. and the nucleotides corresponding to the junction between CD137 and CD3z were entered in the “Overlap Junction List” in order to make sure that either the forward or reverse primers would overlap this junction. This resulted in four primers and probe pairs (see Table 1). Two pairs have the forward primer spanning the CD137/CD3z junction and two pairs have the reverse primer spanning the junction. The assay design parameters were then adjusted to restrict the probe design to span the CD137/CD3z junction. This resulted in three additional primers and probe pairs (see Table 1) for a total of seven primers and probe pairs suitable for testing for qPCR method development. All 7 primers/probe pairs were put through the NCBI BLAST site to check for the potential for cross reactivity in the human genome. None of the BLAST results for any of the 7 pairs indicated a potential for cross reactivity.

TABLE 1
Primers and Probe Pairs Targeting the
CD137/CD3z Junction

| | | | PCR |
|---|---|---|---|
| | | | Product |
| Oligo Set | | | Size |
| Name | Oligo | Sequence (3′-5′) | (bp) |
| Transgene | Forward | GGATGTGAACTGAGAGTGAA | 104 |
| (FP) Set 1 | | G (SEQ ID NO: 5) | |
| | Reverse | TCCTCTCTTCGTCCTAGATT | |
| | | G (SEQ ID NO: 6) | |
| | Probe | TTATAGAGCTGGTTCTGGCCCTG | |
| | | C (SEQ ID NO: 4) | |
| Transgene | Forward | TGAACTGAGAGTGAAGTTCAGC | 93 |
| (FP) Set 2 | | A (SEQ ID NO: 2) | |
| | Reverse | CTTCGTCCTAGATTGAGCTCG | |
| | | T (SEQ ID NO: 3) | |
| | Probe | AGCAGGGCCAGAACCAGCTCTAT | |
| | | A (SEQ ID NO: 1) | |
| Transgene | Forward | GGCAGAAAGAAACTCCTGTA | 129 |
| (RP) Set 1 | | T (SEQ ID NO: 8) | |
| | Reverse | CTTCACTCTCAGTTCACATC | |
| | | C (SEQ ID NO: 9) | |
| | Probe | TCTTCTGGAAATCGGCAGCTACA | |
| | | GC (SEQ ID NO: 7) | |
| Transgene | Forward | CCAGTACAAACTACTCAAGAG | 90 |
| (RP) Set 2 | | G (SEQ ID NO: 11) | |
| | Reverse | GCTGAACTTCACTCTCAGT | |
| | | T (SEQ ID NO: 12) | |
| | Probe | TCTTCTGGAAATCGGCAGCTACA | |
| | | GC (SEQ ID NO: 10) | |
| Transgene | Forward | CTGCCGATTTCCAGAAGAA | 132 |
| (PRB) Set 1 | | G (SEQ ID NO: 14) | |
| | Reverse | TCCTCTCTTCGTCCTAGATTG | |
| | | (SEQ ID NO: 15) | |
| | Probe | AGAAGGAGGATGTGAACTGAGAG | |
| | | TGAAGT (SEQ ID NO: 13) | |
| Transgene | Forward | CTGTAGCTGCCGATTTC | 145 |
| (PRB) Set 2 | | C (SEQ ID NO: 17) | |
| | Reverse | ATCGTACTCCTCTCTTCGT | |
| | | C (SEQ ID NO: 18) | |
| | Probe | AGGAGGATGTGAACTGAGAGTGA | |
| | | AGT (SEQ ID NO: 16) | |
| Transgene | Forward | CTGCCGATTTCCAGAAGAA | 132 |
| (PRB) Set 3 | | G (SEQ ID NO: 20) | |
| | Reverse | TCCTCTCTTCGTCCTAGATT | |
| | | (SEQ ID NO: 21) | |
| | Probe | AGGAGGATGTGAACTGAGAGTGA | |
| | | AGT (SEQ ID NO: 19) | |

Note:
FP = forward primer spans junction;
RP = reverse primer spans junction;
PRB = probe spans junction

Three hALB primers and probe sets were used to test in qPCR method development (see Table 2). One set was taken from a published paper (S Charrier et al. Lentiviral vectors targeting WASp expression to hematopoietic cells, efficiently transduce and correct cells from WAS patients. Gene Therapy (2007) 14, 415-428.), a second set was taken from a CRO digital PCR method assay that was not ultimately used, and a third set was designed using the PrimerQuest Tool and the hALB gene region targeted by both the published paper as well as the CCHMC hALB primers and probe sets. All 3 primers/probe pairs were put through the NCBI BLAST site to check for the potential for cross reactivity with any non-hALB product in the human genome. None of the BLAST results for any of the 3 pairs indicated a potential for cross reactivity.

TABLE 2
hALB Primers and Probe Pairs

| | | | PCR |
|---|---|---|---|
| | | | Product |
| Oligo Set | | | Size |
| Name | Oligo | Sequence (3′-5′) | (bp) |
| hALB Set 1 | Forward | TCATCTCTTGTGGGCTGTAATC | 123 |
| | | (SEQ ID NO: 23) | |
| | Reverse | TGCTGGTTCTCTTTCACTGAC | |
| | | (SEQ ID NO: 24) | |
| | Probe | AGGGAGAGATTTGTGTGGGCATG | |
| | | AC (SEQ ID NO: 22) | |
| hALB Set 2 | Forward | GCTGTCATCTCTTGTGGGCTGT | 139 |
| | | (SEQ ID NO: 26) | |
| | Reverse | ACTCATGGGAGCTGCTGGTTC | |
| | | (SEQ ID NO: 27) | |
| | Probe | CCTGTCATGCCCACACAAATCTC | |
| | | TCC (SEQ ID NO: 25) | |
| hALB Set 3 | Forward | CTGTCATGCCCACACAAA | 95 |
| | | (SEQ ID NO: 29) | |
| | Reverse | ATAAGGCTATCCAAACTCATGG | |
| | | (SEQ ID NO: 30) | |
| | Probe | CCCTGGCATTGTTGTCTTTGCAG | |
| | | A (SEQ ID NO: 28) | |

Note:
Set 1 = CCHMC hALB set;
Set 2 = Published paper hALB set;
Set 3 = Internally designed hALB set

Screening Primers and Probes:

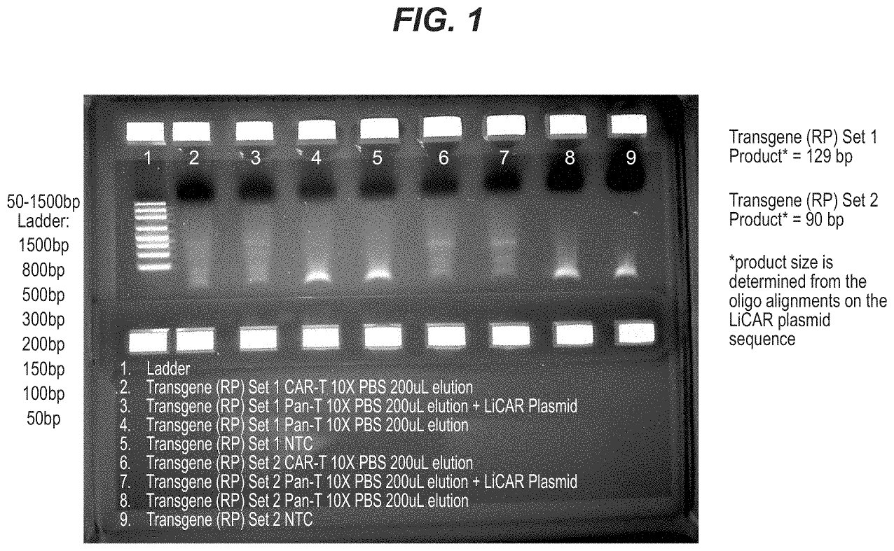

The 7 different Transgene primers and probe sets and the 3 different hALB primers and probe sets were screened by running singleplex qPCR reactions with the CAR T drug product, mock T-cell DNA as samples spiked with pLLV-LICAR2SIN plasmid, and mock T-cell DNA. The mock T-cell DNA was harvested from T-cells that had gone through the same selection and amplification process as the CAR T product does before transduction with the lentivector. The qPCR products of the CAR T cell, mock T-cell, mock T-cell DNA spiked with pLLV-LICAR2SIN plasmid, and the “no template control” (NTC) sample qPCR products were then run out on an agarose gel. Any primers/probe set that did not produce a single band of expected PCR product size were excluded from further transgene qPCR method development. The presence of an ˜25 bp primer dimer band was seen in all qPCR products as well, including the NTC sample, but this band is an expected bi-product of the qPCR reaction. Only the Transgene (FP) Set 1 and Transgene (RP) Set 2 primers/probe sets produced the expected target band with only two <50 bp bands seen on the gel. One of the <50 bp band was likely expected primer dimers. The second <50 bp band could not clearly be seen in the NTC sample and it was undetermined what this additional low molecular weight band may be the result of See FIG. 1 for examples of a gel image from the singleplex primers/probe screening assays. Only the hALB Set 3 primers/probe set was eliminated from further qPCR method development due to additional unexpected bands seen in the qPCR products of CAR T, mock T-cell and mock T-cell DNA spiked with plasmid samples. All 3 hALB primers/probe sets also resulted in two <50 bp bands seen on the gel. One of the <50 bp band was likely expected primer dimers. The second <50 bp band seen was not clearly seen in the NTC samples and it was undetermined what this additional low molecular weight band may be the result of. However, with this being the same low molecular weight banding pattern seen with all the Transgene primers/probe sets as well, and given that optimizing the amount of primers or probe in the reactions and testing different annealing temperatures did not remove the band, it was expected that this additional band is the result of having uracil-DNA glycosylases (UNG) in the qPCR master mix or some other irrelevant qPCR bi-product.

The next step in the Transgene primers/probe sets screening process was to run the 2 acceptable sets (FP Set 1 and RP Set 2) in multiplex qPCR reactions with the two acceptable hALB primers/probe sets (Set 1 and Set 2) using a standard curve. The standard curve was made by spiking mock T-cell DNA with a known concentration of pLLV-LICAR2SIN plasmid and making five, 5-fold serial dilutions of this spiked mock T-cell sample using low EDTA TE buffer as the diluent. Each of the standard curve points were made and frozen in single use aliquots. Both transgene primers/probe sets were first tested with hALB Set 2 in multiplex qPCR. In addition, a CAR T DNA and mock T-cell DNA sample were run to access specificity of the multiplex reaction. The criteria the standard curve was expected to meet for both the transgene and hALB targets to be acceptable for further transgene qPCR method development were as follows: (1) R² of ≥0.98 and (2) qPCR efficiency of 90-110%. The CART DNA was required to have measurable amplification in both the transgene and hALB targets while the mock T-cell DNA sample was required to have only measurable amplification in the hALB target and no amount of amplification in the transgene target to meet the requirements for assay specificity.

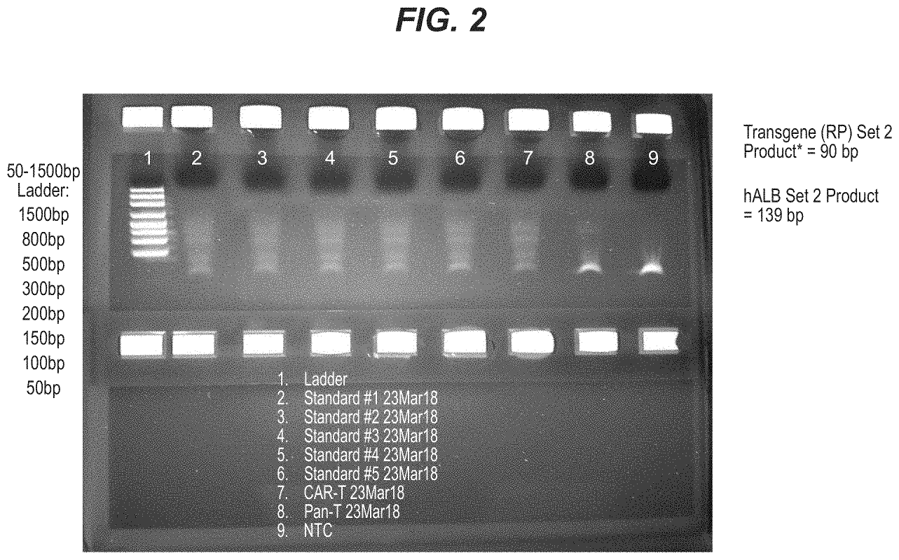

The Transgene (FP) Set 1 and hALB Set 2 multiplex reaction gave acceptable R² results of >0.98 for both the transgene and hALB targets, but neither target standard curve resulted in qPCR efficiencies within the acceptable range. The Transgene (RP) Set 2 and hALB Set 2 multiplex reaction also gave acceptable R² results of >0.98 for both the transgene and hALB targets. The transgene standard curve also resulted in a qPCR efficiency within the acceptable range, but the hALB standard curve did not. Both the Transgene (FP) Set 1/hALB Set 2 and Transgene (RP) Set 2/hALB Set 2 multiplex reactions gave acceptable specificity results with the CAR T DNA sample having measurable amplification in both targets and the mock T-cell DNA having only measurable amplification in the hALB target with no amplification seen in the transgene target. The multiplex qPCR products were also run out on a gel to determine if there were any off-target bands when the two primers/probe sets were multiplexed (see FIG. 2 for an example gel image). No unexpected bands were seen in the gel results for either multiplex reaction. It was decided to test the standard curve in singleplex reactions to determine if multiplexing the reaction was potentially affecting the qPCR efficiency. Given that only the efficiency of the Transgene (RP) Set 2 reaction was within the acceptable range in the multiplex reaction, only the Transgene (RP) Set 2 and hALB Set 2 primers/probe sets were tested in singleplex. The Transgene (RP) Set 2 singleplex reaction resulted in a lower qPCR efficiency that was outside the acceptable range. The hALB Set 2 singleplex reaction resulted in a higher qPCR efficiency that was within the acceptable range. Neither attempting to optimize the primers/probe concentrations of both target oligo sets nor trying a higher annealing temperature improved the efficiencies of either target standard curve in the multiplex reactions. Both acceptable Transgene primers/probe sets were then run in multiplex reactions with the hALB Set 1 primers/probe.

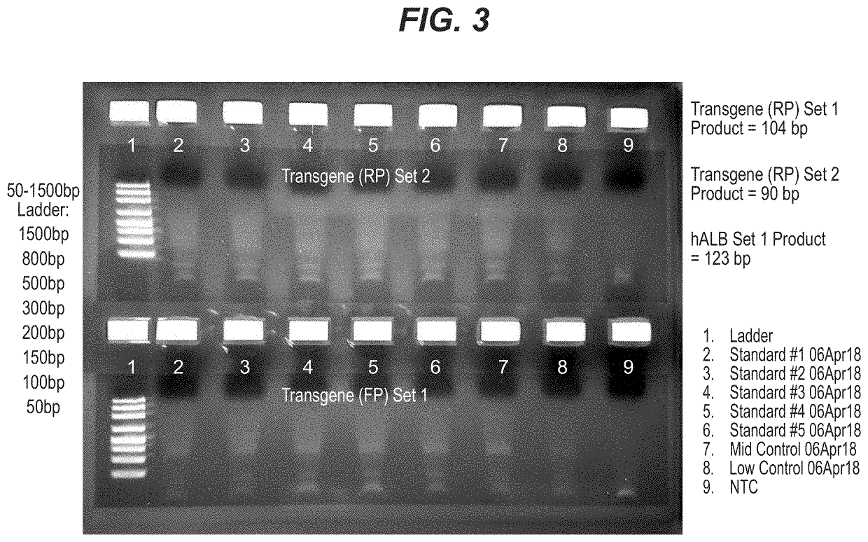

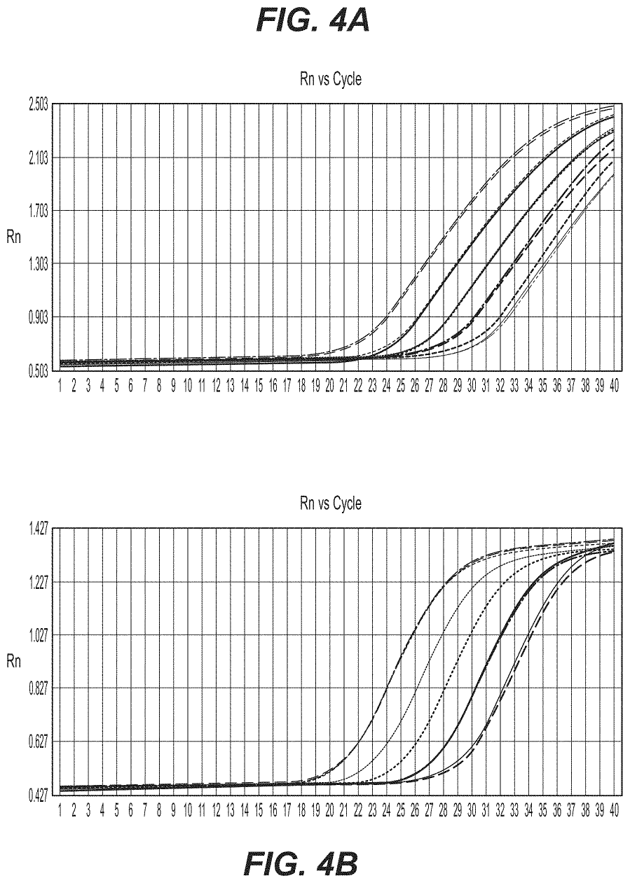

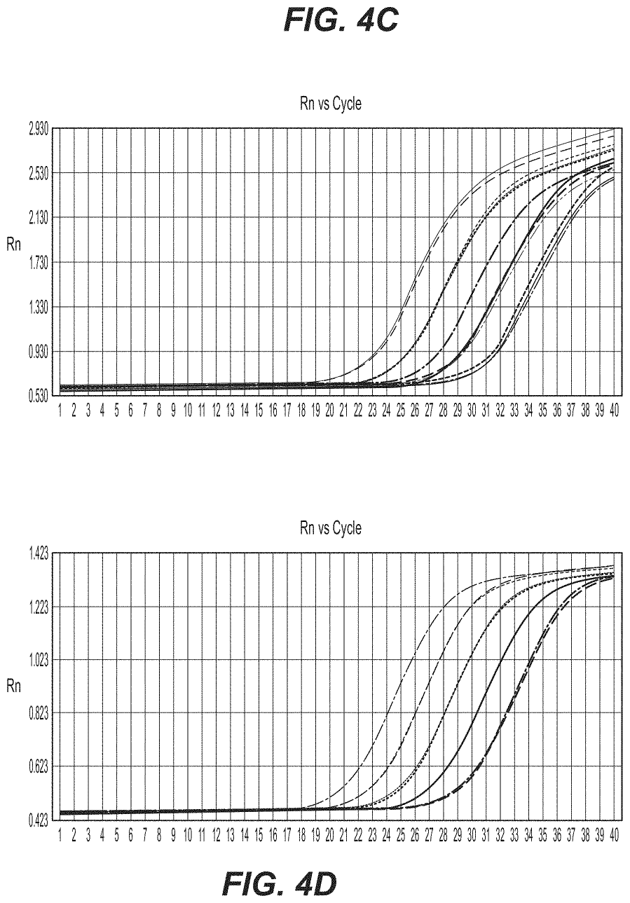

The Transgene (FP) Set 1 and hALB Set 1 multiplex reaction was tried first and had an R²>0.98 for only the hALB standard curve. The Transgene target standard curve R² was <0.97. Both the Transgene and hALB target standard curves resulted in efficiencies outside the acceptable range and the Transgene target had a lower efficiency than that seen for the multiplex reaction with the hALB Set 2 primers/probe. The hALB Set 1 singleplex reaction had an R² of ≥0.98 and resulted in a similar efficiency to that seen in the multiplex reaction. A new standard curve was made using a 5-point, 4-fold dilution scheme and contained a lower amount of pLLV-LICAR2SIN plasmid and mock T-cell DNA in Standard #1. This was done in an attempt to improve the qPCR efficiencies by potentially diluting out any possible PCR inhibitors that may be present in the mock T-cell DNA stock as well as lowering the amount of mock T-cell DNA needed to make larger lots of standards. Both the acceptable transgene primers/probe sets and the hALB Set 1 primers/probe set were then tested in multiplex reactions using this new standard curve. The R² and efficiencies of both the transgene and hALB standard curves were well within the acceptable range for both the Transgene (FP) Set 1/hALB Set 1 multiplex reaction and Transgene (RP) Set 2/hALB Set 1 multiplex reaction using the new standard curve. The multiplex qPCR reaction products were also run out on a gel to ensure no off-target bands were detected (see FIG. 3). No off-target bands were detected for either multiplexed reaction. While the efficiencies were similar between the multiplex reactions of the two transgene primers/probe sets, the shape of the transgene target amplification curves for the Transgene (RP) Set 2/hALB Set 1 multiplex reaction was more of a typical sigmoidal curve, having a more defined upper plateau than that of the Transgene (FP) Set 1/hALB Set 1 multiplex reaction (see FIG. 4). Therefore, the Transgene (RP) Set 2 and hALB Set 1 primers/probe sets were selected for further transgene qPCR method development.

Troubleshooting Efficiency Repeatability:

The Transgene (RP) Set 2/hALB Set 1 multiplex reaction using the new standard curve scheme was repeated to determine if the acceptable results for R² and efficiencies were repeatable. However, the standard curve for both the Transgene and hALB targets were only 88% and 89%, respectively, for the repeat assay. Attempting to optimize the primers/probe concentrations for both targets slightly improved the efficiency for both targets but trying to increase the annealing temperature and trying PCR enhancers DMSO, TMAC and betaine did not. At the same time, the repeatability of the efficiency results was being investigated, it was asked if the VCN/cell range covered by the 4-fold standard curve could be increased in order to lower the potential LOQ of the assay. Multiplex reactions were, therefore, run using the five frozen 4-fold standard point samples as well as making a five point, 5-fold standard curve by diluting the frozen standard #1. The two standard curves were then run side-by-side in a multiplex qPCR reaction. The 4-fold standard curve resulted in efficiencies of 94% and 91% for the transgene and hALB targets, respectively. The 5-fold standard curve resulted in efficiencies of 102% and 99% for the Transgene and hALB targets, respectively. It was thought that the increase in efficiencies seen in the 5-fold vs 4-fold standard curve may be due to an increased variability in the lower standard curve points when those lower standard points are frozen vs diluting a frozen standard #1 to make standards #2-5 fresh just prior to running in the curve in an assay.

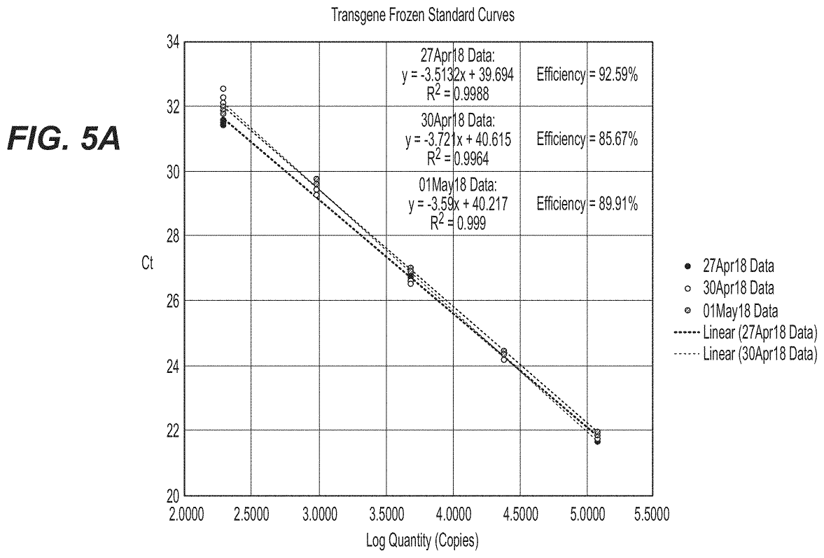

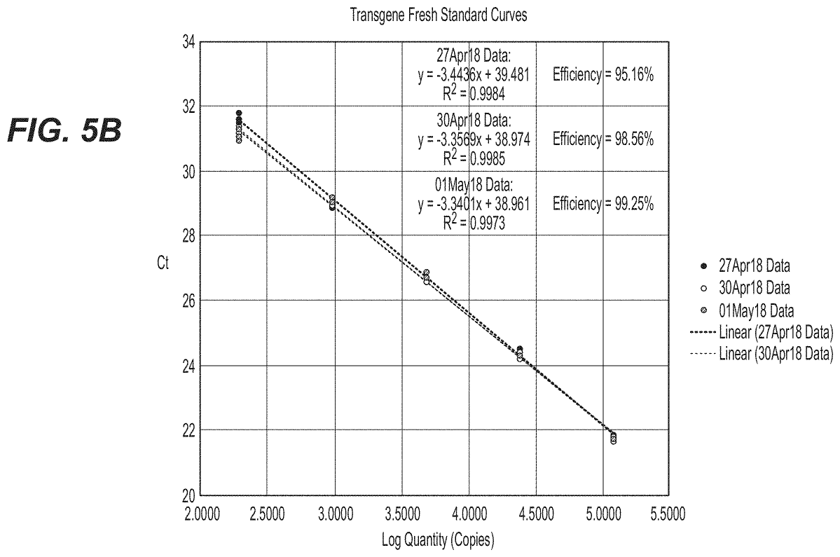

The 4-fold and 5-fold standard curves were repeated to see if the efficiencies still showed an improvement with the 5-fold, “fresh” standard curve vs the 4-fold, “frozen” standard curve points. For the repeat assay, the 4-fold, “frozen” standard curve resulted in efficiencies of 94% and 88% for the transgene and hALB targets, respectively. The 5-fold, “fresh” standard curve resulted in efficiencies of 102% and 99% for the transgene and hALB targets, respectively. Addition runs were performed to further ensure the repeatability of these “fresh” vs “frozen” standard curve results (see FIG. 5). It was determined that the main cause of the variability issues seen in standard curve efficiencies was due to using frozen standard curve points. Therefore, it was decided that only standard #1 would be made and frozen in single use aliquots. This frozen standard #1 would be used to make standards #2-5 fresh just prior to running any assay going forward. It was also determined that the 5-fold standard curve would be used going forward to increase the VCN/cell range of the assay.

Troubleshooting VCN/Cell Discrepancy with DDPCR

Assays were run to collect data as well as to release at least the first 6 batches of material. The VCN/cell assay run targets the RU5 promoter regions of the pLLV-LICAR2SIN plasmid backbone [INVENTORS: Is more detail needed to describe these regions are would this be sufficiently specific to one skilled in the art?] The transgene qPCR method is intended to replace this backbone method as it is a regulatory requirement that the VCN/cell qPCR assay target the transgene portion the CAR plasmid for cell therapies. Therefore, it was a requirement that the method VCN/cell results be comparable to the transgene qPCR method results. Genomic DNA from CAR T was tested in the transgene qPCR method and compared to the results of the LB_12 sample. The transgene standards and controls were run in ddPCR to determine if the transgene and hALB copy values assigned were correct. The LB_12 DNA sample was also run in ddPCR to determine the true VCN/cell value.

The ddPCR reaction used the Transgene (RP) Set 2 and hALB Set 1 primers/probe in the BioRad Supermix for Probes ddPCR master mix. The thermocycling conditions used were those recommended in the Supermix kit. It is recommended that DNA be enzyme digested to obtain the most accurate ddPCR results, so EcoRI was added to the master mix. EcoRI was confirmed to only cut the pLLV-LICAR2SIN plasmid once and did not cut in the amplification region of either the transgene or hALB targets. The ddPCR results confirmed that the transgene standards and controls transgene and hALB copies were correct but the LB_12 result was more comparable to the RU5 VCN/cell result. It was unknown how the transgene standards and controls could be correct while the VCN/cell results for LB_12 qPCR results were determined to be inaccurate by ddPCR. Therefore, enzymes that cut the pLLV-LICAR2SIN plasmid more than once were used considering that smaller DNA pieces of the LB_12 gDNA might yield more accurate results. Two additional enzymes were tried, one that cut twice and one that cuts three times, but the VCN/cell ddPCR results did not change. Therefore, some CAR T DNA samples and the two transgene controls were enzyme digested, cleaned up the reactions and run the DNA in the transgene qPCR along with undigested standards, controls and CAR T samples. The VCN/cell results of the digested controls were ˜3.8 fold higher than those of the undigested VCN/cell results while the digested CAR T samples VCN/cell results were ˜1.3 fold lower than that for the undigested CAR T samples. These results called into question whether the pLLV-LICAR2SIN plasmid needed to be linearized in order to obtain accurate sample VCN/cell results.

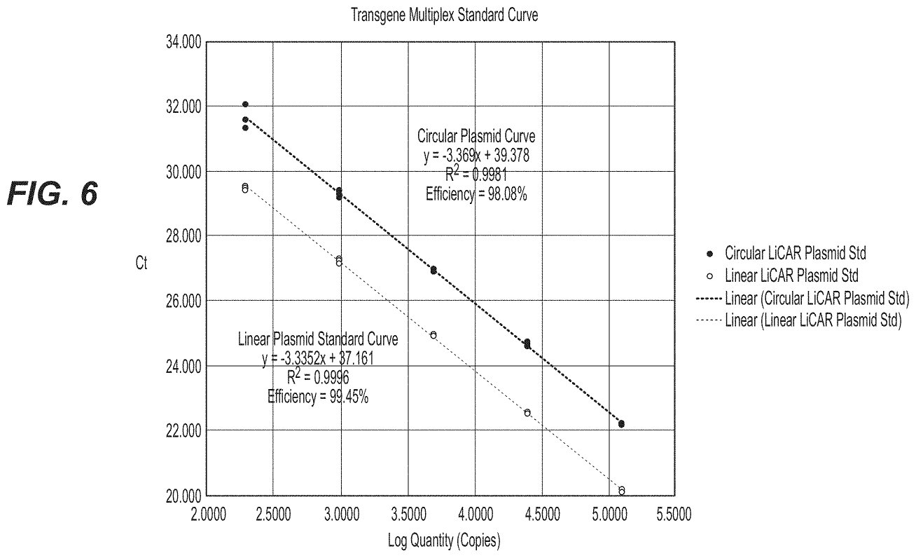

An aliquot of pLLV-LICAR2SIN plasmid was linearized by digesting the plasmid using the EcoRI enzyme. The enzyme digestion was cleaned up and the linearized plasmid quantified. This linearized plasmid was diluted to the transgene copy values of the 5-fold transgene standard curve and run in the transgene qPCR assay. The LB_12 CAR T DNA was also run in the assay and the VCN/cell results were calculated from both the circular (undigested) and linearized standard curve results. The Ct values for the linearized standard curve points were ˜2 fold lower than those of the undigested standard curve points (see FIG. 6). In addition, the LB_12 VCN/cell results calculated from the linearized standard curve were comparable to those obtained in ddPCR as well as those obtained by an additional RU5 qPCR method while the VCN/cell results calculated from the circular (undigested) standard curve were ˜4 fold higher. This confirmed the need to linearize the pLLV-LICAR2SIN plasmid in order to obtain accurate VCN/cell results. Two lots of linearized plasmid standard and controls were made, one large lot to be used as a GMP lot for clinical batch release testing and any other GMP study and one smaller lot to be used for analyst training and any non-GMP activity.

Linearity in a Constant Amount of DNA Typical of a Sample:

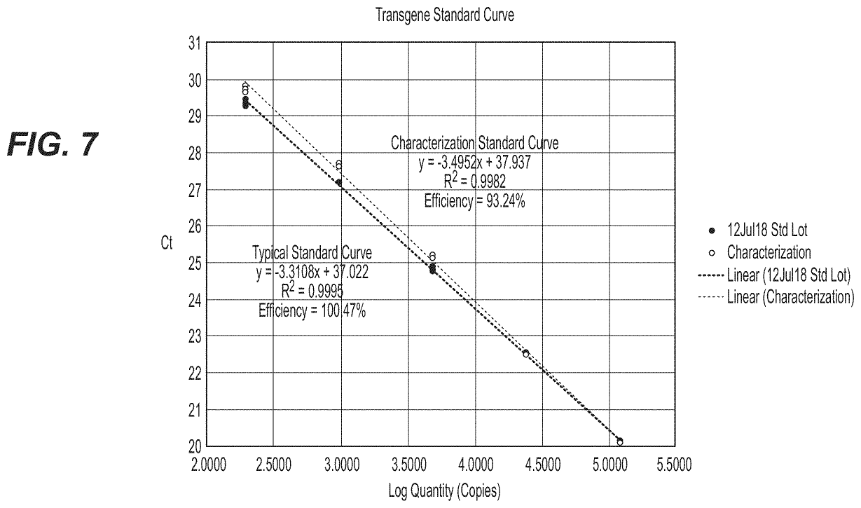

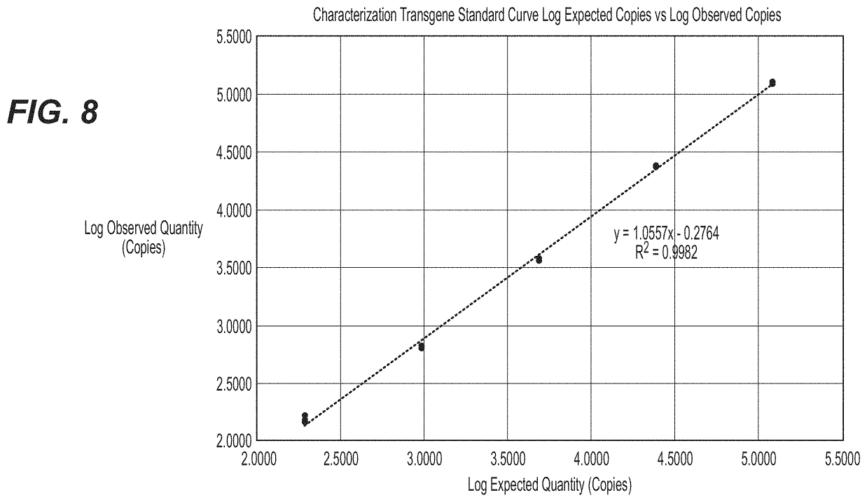

Sample DNA is diluted to a concentration of 0.02 ug/uL and 5 uL are loaded into the qPCR assay for a total of 100 ng of DNA per reaction. Samples with stock concentrations of <0.02 ug/uL can be run straight in the assay, but the acceptance range for hALB copies must be adjusted based on the amount of DNA loaded on the reactions. The standard curve is made with a starting mock T-cell DNA concentration of 0.05 ug/uL and diluted with low EDTA TE buffer in order to achieve a standard curve for both the Transgene and hALB targets (see Table 3). To ensure the linearity of the assay remains within the acceptable range if the standard curve was made in a constant amount of mock T-cell gDNA typical of a sample, a characterization standard curve was made using a mock T-cell DNA concentration of 0.02 ug/uL and serially diluted using 0.02 ug/uL mock T-cell DNA (see Table 4). This standard curve was then run side-by-side with the typical standard curve to ensure linearity of the assay (see FIG. 7). The log Observed Copies vs log Expected Copies were also plotted to ensure the measured transgene copy results for the characterization standard curve resulted in a linear response with an R² of ≥0.98 (see FIG. 8).

The characterization standard curve results showed that the Transgene copies could still be accurately quantified in a DNA concentration consistent with a typical sample concentration (0.02 ug/uL).

TABLE 3
Transgene qPCR Standard Curve

| | Volume of | | | | |
|---|---|---|---|---|---|
| | Previous | Volume of | | | |
| | Standard | TE Buffer | Fold | Transgene | hALB |
| Standard# | (uL) | (uL) | Dilution | Copies | Copies |
| 1 | N/A | N/A | N/A | 121212.121 | 75757.576 |
| 2 | 5 | 20 | 5 | 24242.424 | 15151.515 |
| 3 | 5 | 20 | 5 | 4848.485 | 3030.303 |
| 4 | 5 | 20 | 5 | 969.697 | 606.061 |
| 5 | 5 | 20 | 5 | 193.939 | 121.212 |

TABLE 4
Characterization Transgene qPCR Standard Curve

| | Volume of | Volume of | | | |
|---|---|---|---|---|---|
| | Previous | 0.02 ug/uL | | | |
| | Standard | mock DNA | Fold | Transgene | hALB |
| Standard# | (uL) | (uL) | Dilution | Copies | Copies |
| 1 | N/A | N/A | N/A | 121212.121 | 30303.030 |
| 2 | 5 | 20 | 5 | 24242.424 | 30303.030 |
| 3 | 5 | 20 | 5 | 4848.485 | 30303.030 |
| 4 | 5 | 20 | 5 | 969.697 | 30303.030 |
| 5 | 5 | 20 | 5 | 193.939 | 30303.030 |

Method Qualification:

The transgene qPCR method was qualified according to International Conference on Harmonization (ICH) and MIQE (minimum information for publication of quantitative real-time PCR experiments) guidelines. Three assays were run to complete method qualification. The assay passed the acceptance criteria for all method qualification parameters specified in the method qualification protocol. Table 5 summaries the method qualification parameters, acceptance criteria and the qualification results (see Example 2).

TABLE 5
Summary Transgene Multiplex qPCR Method Qualification

| Parameter | Acceptance Criteria | Results |
|---|---|---|
| Repeatability | The % CV of the triplicate VCN/cell results | Mid Control (2.00 |
| | for the mid and low assay controls within | VCN/cell): 4-6% |
| | each valid qualification assay must be ≤30%. | Low Control (0.20 |
| | | VCN/cell): 4-6% |
| Intermediate | The % CV of the VCN/cell results for the | Mid Control (2.00 |
| Precision | mid and low assay controls across all | VCN/cell): 4% |
| | valid qualification assays must be ≤30%. | Low Control (0.20 |
| | | VCN/cell): 6% |
| Specificity | All replicate Ct values for the Mock T-cell | Transgene target: All |
| (Mock T-cell | DNA must be “Undetermined” for the | replicate Ct values |
| DNA) | Transgene target in addition having | were “Undetermined” |
| | mean hALB copies within 21,212-39,394 | in each assay. |
| | copies for each valid qualification | hALB target: mean |
| | assay. | hALB copies ranged |
| | | from 28,719-29,611. |
| Specificity | All replicates of the CAR T DNA must | Transgene target: all |
| (CAR T DNA) | have a quantifiable Transgene result in | copy values were |
| | addition having mean hALB copies within | quantifiable and ranged |
| | 21,212-39,394 copies for each valid | from 4,592.801- |
| | qualification assay. | 5,153.907. |
| | | hALB target: mean |
| | | hALB copies ranged |
| | | from 31,552-33,725. |
| Range | The range is defined as the copy range | Range: 193.939- |
| (Transgene | covered by the 5-point standard curve | 121212.121 copies |
| Target) | provided the Transgene target satisfies all | |
| | criteria for accuracy, linearity and | |
| | intermediate precision. | |
| Range (hALB | The range is defined as the copy range | Range: 121.212- |
| Target) | covered by the 5-point standard curve | 75757.576 copies |
| | provided the hALB target satisfies all | |
| | criteria for accuracy, linearity and | |
| | intermediate precision. | |
| LOQ (Transgene | LOQ is defined as the Transgene copy | LOQ: 0.02 VCN/cell |
| Target) | result for the lowest LOQ sample to have | LOQ sample Transgene |
| | % CV ≤20% for both the mean Transgene | copies of 303.030. |
| | copy result and mean VCN/cell results as | |
| | well as % recovery within 70-130% for | |
| | both the mean Transgene copy result and | |
| | mean VCN/cell result for each valid | |
| | qualification assay. | |
| LOQ (hALB | LOQ is defined as the copy value of | LOQ: 121.212 copies |
| Target) | Standard #5 provided the hALB | |
| | target satisfies all criteria for | |
| | accuracy, linearity and intermediate | |
| | precision. | |

### EXAMPLE 2

### Transgene qPCR Method

1.0 Purpose

1.1 This example describes an example procedure for performing the quantitative real time PCR (qPCR) assay for the quantitation of the LiCAR plasmid integrated into CAR T product. The assay is designed as a multiplex qPCR where the junction between the CD137 and CD3z regions of the LiCAR plasmid as well as human albumin (reference gene) are targeted.

2.0 Scope

2.1 This method is applicable to post-harvest CAR T cells, just prior to dose formulation for determination of:
2.1.1 Vector Copy Number
2.1.2 Transduction Efficiency
2.1.3 LiCAR Expression Identity

3.0 Definitions and Abbreviations
3.1 LOQ (Limit of Quantitation)
3.2 NTC (No Template Control)
3.3 Ct (Cycle Threshold)
3.4 CV (Coefficient of Variation)
3.5 SD (Standard Deviation)
3.6 hALB (human Albumin)
3.7 BCMA (B-cell Maturation Antigen)
3.8 VCN (Vector Copy Number)

4.0 Equipment
4.1 Centrifuge capable of spinning 96 well PCR plates (For example: Beckman Coulter, Allegra X-14R with SX4750 rotor and swing set for 96 well plate)
4.2 QuantStudio 6 Real-Time PCR System
4.3 Freezer capable of −70° C.
4.4 Freezer capable of −20° C.
4.5 Calibrated 8 or 12 Channel Pipettes (20, 50 uL or other appropriate size), for example: Rainin pipettes
4.6 Calibrated single Channel Pipettes (20, 100, 200, 1000 uL or other appropriate size), for example: Rainin pipettes
4.7 Refrigerator or cold room capable of maintaining 2-8° C.
4.8 QuantStudio PCR Software v1.3 or greater
4.9 Vortex mixer
4.10 Biosafety Cabinet

5.0 Materials
Note: Materials designated “for example” may be substituted by similar materials
without prior qualification. For materials designated “or equivalent”, alternatives must be demonstrated to be equivalent prior to use for testing samples.
   5.1 DNase/RNase-Free Water, for example: Invitrogen Cat #10977015.
   5.2 TaqPath ProAmp Master Mix, ThermoFisher Cat #A30866, or equivalent.
   5.3 Qualified Lot of BCMA Transgene and ALB Primers and Probe, custom sequences through IDT or equivalent.
   5.4 Qualified Lot of BCMA Transgene Standard #1
   5.5 Qualified Lot of BCMA Transgene Mid and Low Controls
   5.6 1× Low EDTA TE Buffer pH 8.0, RNase/DNase free, for example: Quality Biological Cat #351-324-721.
   5.7 0.5 mL Centrifuge tubes, sterile, RNase/DNase free, for example: Eppendorf Cat #022431005
   5.8 1.5 mL Centrifuge tubes, sterile, RNase/DNase free, for example: Eppendorf Cat #022431021
   5.9 2 mL Centrifuge tubes, sterile, RNase/DNase free, for example: Eppendorf Cat #022431048
   5.10 5 mL Centrifuge tubes, sterile, RNase/DNase free, for example: Eppendorf Cat #0030119460
   5.11 Pipette tips, sterile, filtered, (20, 200, 1000 uL or other appropriate size), for example: Rainin Cat #30389226, 30389240, 30398213
   5.12 96 well PCR plates, Applied Biosystems, Cat #4483343, 4483354, 4483349, 4483350, 4483395 or equivalent
   5.13 Micro Amp Optical Adhesive Film, Applied Biosystems, Cat #4311971, or equivalent
   5.14 Reagent reservoirs, sterile, RNase/DNase free, for example: VistaLabs Cat #3054-1002

6.0 Precautions
6.1 Wear appropriate PPE when working in the laboratory.
6.2 Follow site specific guidelines for working with hazardous chemicals. Consult manufacturer's SDS for more details.
6.3 All pipetting steps must be performed using aseptic technique in a BSC. It is recommended that analysts wear disposable sleeve covers during all steps of the procedure to minimize the risk of contamination to any of the materials or reagents.

7.0 Procedure
7.1 Obtain an aliquot of each of the following:
   7.1.1 BCMA Transgene Forward Primer 10 μM working stock
   7.1.2 BCMA Transgene Reverse Primer 10 μM working stock
   7.1.3 BCMA Transgene Probe 10 μM working stock
   7.1.4 hALB Forward Primer 10 μM working stock
   7.1.5 hALB Reverse Primer 10 μM working stock
   7.1.6 hALB Probe 10 μM working stock
   7.1.7 Qualified BCMA Transgene qPCR Standard #1
   7.1.8 Qualified BCMA Transgene qPCR 2.00 copies/cell mid control
   7.1.9 Qualified BCMA Transgene qPCR 0.20 copies/cell low control
7.2 Prepare the multiplex master mix for the appropriate number of reactions according to Table 6. Additional excess reactions can be added by including more samples than will actually be run in the assay. For example, if 10 samples will be run, but more than the 10 excess reactions that are already included in the math of table below are desired, indicate at 11 samples will be run (i.e. N=11). Each additional sample will add the volume of 3 reactions.

TABLE 6
Master Mix Composition

| | | Final |
|---|---|---|
| | | Concentration. in |
| | Volume for N Samples | 25 μL Total |
| Reagent | (μL)* | Reaction |
| TaqPath ProAmp Master Mix | 12.5 × (34 + 3N) | 1X |
| DNase/RNase Free Water | 5.625 × (34 + 3N) | N/A |
| Transgene Forward | 0.25 × (34 + 3N) | 100 nM |
| Primer (10 μM) | | |
| Transgene Reverse | 0.25 × (34 + 3N) | 100 nM |
| Primer (10 μM) | | |
| Transgene FAM | 0.5 × (34 + 3N) | 200 nM |
| Probe (10 μM) | | |
| hALB Forward | 0.1875 × (34 + 3N) | 75 nM |
| Primer (10 μM) | | |
| hALB Reverse | 0.1875 × (34 + 3N) | 75 nM |
| Primer (10 μM) | | |
| hALB HEX Probe (10 μM) | 0.5 × (34 + 3N) | 200 nM |

*Formula is volume of component needed for a single 25 uL reaction multiplied by the sum of 24 standards/control wells plus 10 excess reactions (34) and 3*number of samples (3 reaction wells per sample).

7.3 Briefly vortex mix the master mix tube and set aside.
7.4 Prepare Standards #2-5 using Low EDTA TE buffer according to Table 7. Make sure to briefly mix each dilution prior to moving on to making the next dilution.

TABLE 7
Preparation of 5-Point, 5-Fold Standard Curve

| | Volume of | | | | |
|---|---|---|---|---|---|
| | Previous | Volume of | | | |
| | Standard | TE Buffer | Fold | Transgene | hALB |
| Standard# | (uL) | (uL) | Dilution | Copies | Copies |
| 1 | N/A | N/A | N/A | 121212.121 | 75757.576 |
| 2 | 5 | 20 | 5 | 24242.424 | 15151.515 |
| 3 | 5 | 20 | 5 | 4848.485 | 3030.303 |
| 4 | 5 | 20 | 5 | 969.697 | 606.061 |
| 5 | 5 | 20 | 5 | 193.939 | 121.212 |

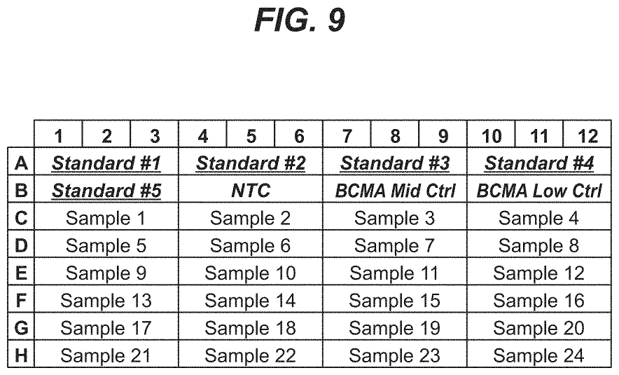

7.5 Briefly vortex mix the master mix solution again and pipette the mix into a reagent reservoir.
7.6 Pipette 20 μL of master mix to the appropriate wells of a 96 well PCR plate using a multichannel pipette (see FIG. 9).
7.7 Load 5 μL of standards, controls, and sample DNA to the appropriate wells of the 96 well PCR plate using a single channel pipette according to the plate layout in FIG. 9. Load 5 uL of low EDTA TE buffer to the NTC wells.
7.8 Seal the plate with the optical adhesive film and centrifuge briefly at ˜300×g.
7.9 Load the PCR plate into the PCR instrument.
7.10 Open the “Assay Template.edt”, select “Save As”, enter an appropriate name for the qPCR experiment and save as a .eds file. Do not save over the template file.
7.11 Enter a name for the experiment in the name section.
7.12 Ensure Experiment Properties are set as specified below and the thermocycling conditions (Run Method tab) are set correctly according to those specified in Table 8 using a 25 uL reaction volume.
   7.12.1 Instrument Type: QuantStudio 6 Flex System
   7.12.2 Block Type: 96-well (0.2 mL)
   7.12.3 Experiment Type: Standard Curve
   7.12.4 Detection Reagent: TaqMan Reagents
   7.12.5 Instrument Properties: Standard

TABLE 8
Thermocycling Conditions

| | | |
|---|---|---|
| Stage 1 | Stage 2 | Stage 3 (40 Cycles) |

| | | | | | | | |
|---|---|---|---|---|---|---|---|
| 50° | C. | 95° | C. | 95° | C. | 60° | C. |
| 2 | mins | 10 | mins | 15 | Sec | 1 | min |

| | | | |
|---|---|---|---|
| UNG | Polymerase | Denaturation/Melt | Anneal/Extend |
| activation | Activation | | |

7.13 Select run and the click the instrument serial number to run the assay.

8.0 Data Analysis
8.1 Use the auto baseline and auto Ct feature of the software to analyze the data by clicking Analyze. Then click save to save the analysis.
8.2 Print a report PDF and include this in the assay documentation.
8.3 Calculate the VCN/cell for each sample and positive control triplicate as follows.

$$VCN / cell = ( \frac{Transgene Quality}{hALB Quantity} ) * 2$$

8.4 Calculate the mean, standard deviation and % CV for the triplicate VCN/cell values for each sample and positive control.

9.0 Assay Acceptance Criteria
9.1 Assay Acceptance Criteria
   9.1.1 R2 value for both the Transgene and hALB standard curves must be ≥0.97.
   9.1.2 The slope of the standard curve must be between −3.585 and −3.104 (equates to a PCR efficiency of 90.08-109.97%)
   9.1.3 None of the Ct replicates for any of the standards can be “Undetermined.”
   9.1.4 All Ct replicates of the NTC must be “Undetermined” for both the Transgene and hALB targets.
   9.1.5 The mean Ct for Standard #1 must be <23.0 for the Transgene target and <22.0 for the hALB target.
   9.1.6 The Ct SD for each Standard must be ≤0.60 for both the Transgene and hALB targets.
   9.1.7 The average hALB copies for both the Mid and Low Controls must be 30,303.030 copies +/−30% (expected range: 21,212.121-39,393.939 copies).
   9.1.8 The mean VCN/cell result for the 2.00 VCN/cell Mid control and
   0.20 VCN/cell Low control must be +/−35% of the target VCN/cell value for each control.
   9.1.9 The % CV of the for the Mid and Low positive controls VCN/cell replicates must be ≤20%
   9.1.10 If any of the above criteria are not met, the assay is invalid.
9.2 Sample Acceptance Criteria
   9.2.1 The average hALB copies for each sample must be 30,303.030 copies +/−30% (expected range:21,212.121-39,393.939 copies).
      9.2.1.1 If the concentration of the sample gDNA is <0.02 ug/uL, calculate the expected copies of hALB for that sample from the amount of DNA actually loaded into the reactions.
      Example: Concentration of the sample gDNA is 0.01 ug/uL. (5 uL)(0.01 ug/uL)=0.05 ug=50 ng of sample gDNA per reaction (50ng DNA)(1 copy ALB/0.0033 ng gDNA)=15,152 copies ALB Expected range: 10,606-19,697 copies of ALB
   9.2.2 The triplicate hALB target copy values for a sample must be within the Ct range covered by the hALB standard curve. The Ct range is defined as the lowest Ct value of the Standard #1 triplicate and the highest Ct value of the Standard #5 triplicate.
   9.2.3 The triplicate transgene target copy values for a sample must be above the Transgene LOQ copies of 303.030.
      9.2.3.1 If 1 or more replicate of a sample for the Transgene target is lower than the transgene LOQ of 303.030 copies, the sample must be reported as below LOQ.
   9.2.4 If 1 or more replicate of a sample for the transgene target is lower than the lowest transgene Ct value for Standard #1, the sample must be reported as Above Standard Curve Range, Sample Unquantifiable. For example: if the transgene Ct values of a sample are 20.1, 19.9 and 20.2, but the lowest transgene Ct value achieved in Standard #1 is only 20.0, the 19.9 sample replicate cannot be accurately quantified and therefore the sample must be reported as Above Standard Curve Range, Sample Unquantifiable. Notify management and the study director if sample is determined to be unquantifiable.
   9.2.5 The % CV of the sample VCN/cell replicates must be ≤20%.
      Note: % CV is not accessed on samples with replicates below LOQ or for samples determined to be unquantifiable.
   9.2.6 Any sample with all triplicate transgene copy values above LOQ, and meeting all above acceptance criteria will report the average VCN/cell value out to 2 decimal places (example: 2.02 VCN/cell).
   9.2.7 Any sample that does not meet all above acceptance criteria is invalid.

### Genomic DNA Isolation, Qualification and Dilution

1.0 Purpose
1.1 An example procedure for isolating and quantifying gDNA from CAR T samples or mock T-cell suspensions or frozen cell pellets is described.

2.0 Scope
2.1 The procedure is described for isolating gDNA from:
   2.1.1 Post-harvest CAR T cell samples supplied as frozen cell pellets or fresh cell suspension.
   2.1.2 Mock T-cells supplied as frozen cell pellets or fresh cell suspension.

3.0 Equipment
3.1 Centrifuge capable of spinning 1.5 mL and 5 mL microcentrifuge tubes (For example: Beckman Coulter, Allegra X-14R with SX4750 rotor with adaptors for 1.5 mL microcentrifuge tubes)
3.2 Qubit 4 Fluorometer, Invitrogen Cat #Q33226
3.3 Freezer capable of −70° C.
3.4 Freezer capable of −20° C.
3.5 Calibrated single Channel Pipettes (20, 100, 200, 1000 uL or other appropriate size), for example: Rainin pipettes
3.6 Heat block capable of 55° C. and suitable for 1.5 mL and 2 mL microcentrifuge tubes
3.7 Refrigerator or cold room capable of maintaining 2-8° C.
3.8 Vortex mixer
3.9 Biosafety Cabinet

4.0 Materials

Note: Materials designated “for example” may be substituted by similar materials without prior qualification. For materials designated “or equivalent”, alternatives should be demonstrated to be equivalent prior to use for testing samples.
4.1 DNase/RNase-Free Water, for example: Invitrogen Cat #10977015.
4.2 RPMI 1640 media with L-Glutamine and 25 mM HEPES, for example: Corning Cat #10-041-CV
4.3 1× Low EDTA TE Buffer pH 8.0, RNase/DNase free, molecular biology grade, for example: Quality Biological Cat #351-324-721.
4.4 200 Proof (96-100%) Ethanol molecular biology grade, for example: Decon Labs Cat #3616EA
4.5 10× PBS molecular biology grade, for example: Affymetirx Cat #75889
4.6 PureLink Genomic DNA Mini Kit, Invitrogen Cat #K182001
4.7 Qubit™ Assay Tubes, Invitrogen™ Cat #Q32856 (Invitrogen, Carlsbad, Calif.). 4.9 Qubit dsDNA BR Assay Kit, Invitrogen Cat #Q328350
4.9 0.5 mL Centrifuge tubes, sterile, RNase/DNase free, for example: Eppendorf Cat #022431005
4.10 1.5 mL Centrifuge tubes, sterile, RNase/DNase free, for example: Eppendorf Cat #022431021
4.11 2 mL Centrifuge tubes, sterile, RNase/DNase free, for example: Eppendorf Cat #022431048
4.12 5 mL Centrifuge tubes, sterile, RNase/DNase free, for example: Eppendorf Cat #0030119460
4.13 15 mL Conical tubes, sterile, for example: Corning Cat #431470
4.14 Pipette tips, sterile, filtered, (20, 200, 1000 uL or other appropriate size), for example: Rainin Cat #30389226, 30389240, 30398213

5.0 Precautions
5.1 Wear appropriate PPE when working in the laboratory.
5.2 Follow site specific guidelines for working with hazardous chemicals. Consult manufacturer's SDS for more details.
5.2 All pipetting steps must be performed using aseptic technique in a BSC. It is recommended that analysts wear disposable sleeve covers during all steps of the procedure to minimize the risk of contaminating any materials or reagents.

6.0 Procedure
6.1 When opening a new PureLink Genomic DNA mini kit, make sure to add ethanol to the Wash Buffer 1 and Wash Buffer 2 bottles according to the instructions on each bottle label. Mix the bottles well after ethanol addition and mark on each label that ethanol was added. Include initials and date along with the ethanol addition mark. The kit is stable for up to 1 year when all components are stored at room temperature.
6.2 Set a heat block to 55° C. and allow it to reach temperature before beginning DNA extraction.
6.3 DNA extraction is performed using cell pellets. Either fresh cell pellets or cell pellets stored frozen at −70° C. can be used. It is recommended that a minimum of 2×10⁶ cells is extracted per column, however anywhere up to 4×10⁶ viable cells can be extracted per column.
Note: While the manufacturer specification states that up to 5×10⁶ cells can be extracted per column, this method uses the viable cell count to determine the number of cells to carry out DNA isolation. Therefore a 20% buffer is given to allow for the presence of dead cells that will also be present in the cell suspension. Do not exceed the specified 4×10⁶ viable cells per column. If the percent viability is less than 80%, the maximum number of viable cells extracted per column should be lowered to compensated for the >20% dead cells present in the cell suspension.
6.4 Preparing cell pellets from fresh cell suspension.
   6.4.1 At least a 150 uL aliquot of cell suspension is needed to count on the NC-200. The aliquot can be a dilution of the stock cell suspension in RPMI media as needed to stay within the dynamic range of the NC-200 (5.0×10⁴-5.0×10⁶ cell/ml).
   6.4.2 Use a counting protocol to count the cells.
   6.4.3 Using the viable cell count, determine the volume of cells needed to achieve the desired number of cells to extract per PureLink column and aliquot the cell suspension into 1.5 mL or 2 mL microcentrifuge tubes.
      For example: Viable cell count from the NC-200 is 2×10⁷ cells/mL with a percent viability of 88%. The desired number of cells to extract per PureLink column is 4×10⁶ viable cells:

$$( 4 e 6 cells ) ( \frac{1 mL}{20 e 6 cells} ) = 0.2 mL$$

Aliquot 200 uL of the cell suspension into 1.5 mL or 2 mL microcentrifuge tubes.
6.4.3.1 It is not recommended to aliquot less than 100 uL of cells per microcentrifuge tube. If needed, dilute the cells in RPMI media to achieve a cell count that will allow for aliquoting a minimum of 100 uL of cell suspension per tube.
   6.4.4 Centrifuge the tubes at 300×g for 5 min at RT to pellet out the cells.
   6.4.5 Carefully remove and discard the media from the cell pellet(s).
   6.4.6 The cells pellet(s) may be directly used to isolate DNA or stored at −70° C.
6.5 DNA Extraction:
   6.5.1 If extracting from frozen cell pellets, thaw the cell pellets at room temperature before beginning the DNA extraction procedure.
   6.5.2 Dilute 10× PBS buffer to 1× using RNase/DNase free water. 200 uL of 1× PBS is used per cell pellet. Prepare enough 1× PBS to resuspend the total number of cell pellets to be extracted.
   6.5.3 Resuspend each cell pellet in 200 uL of 1× PBS. Pipette mix to ensure the cell pellet is completely resuspended.
   6.5.4 Add 20 uL of Proteinase K to each tube and vortex briefly to mix.
   6.5.5 Add 20 uL of RNase A to each tube and vortex briefly to mix.
   6.5.6 Incubate the tubes for 2 min at room temperature.
   6.5.7 Add 200 uL of PureLink Genomic Lysis/Binding Buffer to each tube and vortex briefly to obtain a homogeneous solution.
   6.5.8 Place the tubes in the pre-warmed 55° C. heat block and incubate for 10 min.
   6.5.9 Once incubation is complete, remove the tubes from the heat block and add 200 uL of 96-100% ethanol to each tube. Briefly vortex mix to yield a homogeneous solution.
      Note: Condensation accumulates in the lid of the tubes during the incubation at 55° C. Be careful when opening the tubes so not to splash the contents from the lid.
   6.5.10 Set up a single PureLink Spin Column in a collection tube for each sample tube. Label the lid of each spin column with a specific sample identifier so that samples are not mixed up inadvertently. For example, spin columns can be labelled with a sample lot#. Do not label the collection tube as collection tubes are discarded periodically throughout the extraction protocol.
   6.5.11 Add the contents of each sample tube from step 5.5.9 to the corresponding labelled spin column.
   6.5.12 Centrifuge the column(s) at 10,000×g for 1 minute at room temperature. During centrifugation, set up new clean collection tubes for each sample.
   6.5.13 After centrifugation, remove the spin column/collection tubes from the centrifuge. Transfer each spin column to a new clean collection tube and discard the old collection tube with the flow through.
   6.5.14 Add 500 uL of Wash Buffer 1 to each spin column.
   6.5.15 Centrifuge the column(s) at 10,000×g for 1 minute at room temperature. During centrifugation, set up new clean collection tubes for each sample.
   6.5.16 After centrifugation, remove the spin column/collection tubes from the centrifuge. Transfer each spin column to a new clean collection tube and discard the old collection tube with the flow through.
   6.5.17 Add 500 uL of Wash Buffer 2 to each spin column.
   6.5.18 Centrifuge the column(s) at maximum speed for 3 min at room temperature. During centrifugation, set up a single 2 mL microcentrifuge tube for each sample.
   6.5.19 After centrifugation, remove the spin column(s)/collection tubes from the centrifuge. Transfer each spin column a 2 mL microcentrifuge tube and discard the old collection tube with the flow through. Note: Do not use a collection tube supplied in the PureLink kit for step 5.5.19. The kit does not supply additional tubes for the added centrifugation step, so 2 mL microcentrifuge tubes must be used.
   6.5.20 Centrifuge the column(s) at maximum speed for 2 min at room temperature to dry the columns. During centrifugation, set up a single 1.5 mL microcentrifuge tube for each sample. Label each tube at a minimum with the sample name and extraction date. These are the tubes the DNA will be eluted in.
   6.5.21 After centrifugation, remove the spin column(s)/tube(s) from the centrifuge. Transfer each spin column the corresponding labelled 1.5 mL microcentrifuge tube and discard the 2 mL microcentrifuge tube with any flow through.
   6.5.22 Add 25 uL of PureLink Genomic Elution Buffer to the columns. Make sure to place the elution buffer onto the silica membrane but do not touch or pierce the membrane with the pipette tip.
   6.5.23 Incubate the columns for 1 min at room temperature.
   6.5.24 Centrifuge the column(s)/tube(s) at maximum speed for 1 min at room temperature to elute the DNA.
   6.5.25 Remove the column(s)/tube(s) from the centrifuge and repeat step 5.5.22-5.5.23 with an additional 25 uL of PureLink Genomic Elution Buffer.
   6.5.26 After incubation, centrifuge the column(s)/tube(s) at maximum speed for 1.5 min at room temperature to elute additional DNA.
   6.5.27 Remove the column(s)/tube(s) from the centrifuge. Remove the column from the 1.5 mL tube and discard the column.
   6.5.28 The eluted DNA can either be stored at −20° C. or immediately quantified and diluted to the working concentration.
      Note: Do not quantify the DNA unless it will be immediately diluted to the working concentration after quantification. If samples are quantified but cannot immediately be diluted to the qPCR working concentration, the samples should be place in −20° C. and must be re-quantified before diluting to the qPCR working concentration after thaw.
6.6 DNA Quantification:
   6.6.1 DNA is quantified using the Qubit dsDNA Broad Range kit and the Qubit 4 Fluorometer. The kit is highly selective for double-stranded DNA (dsDNA) over RNA and is designed to be accurate for initial sample concentrations from 100 pg/uL-1,000 ng/uL. All kit components must be handled in a BSC and handled aseptically to prevent contamination of any kit component.
      Note: The Qubit dsDNA BR Reagent contains DMSO and will freeze at temperatures below RT. Repeated freeze/thaw cycles of the Qubit Reagent must be avoided so the Reagent must be stored at RT. The Qubit Buffer is designed to be stored at RT and is the recommended storage condition. The Qubit Standards must be stored at 2-8° C.
   6.6.2 The Qubit 4 Fluorometer is calibrated using the two standards supplied in the Qubit dsDNA Broad Range kit. The standards need to be prepared and run with each set of DNA samples to be quantitated. Never re-use the calibration from a previous run as the most accurate quantitation is achieved when the standards and DNA samples are prepared using the same Qubit working solution.
   6.6.3 Label 1 Qubit assay tube for each standard and sample to be quantitated. Only label the lids the of tubes. Do not label the sides of the tubes as this will interfere with the quantitation.
      Note: Only Qubit assay tubes can be used in the Qubit Fluorometer. These tubes are specifically designed to give the most accurate results.
   6.6.4 Prepare Qubit working solution by diluting the Qubit dsDNA BR Reagent 1:200 in Qubit dsDNA BR Buffer. Make sure to prepare sufficient working solution to accommodate both standards and all samples to be quantitated. The minimum volume needed is equal to (2 (2 standards)+# of samples to be quantitated+1)*200.
      For example: To quantitate 8 samples, prepare enough working solution for the samples and 2 standards plus at least one additional sample for overage. Assume 200 uL of working reagent per tube in 11 tubes (8+2+1=11 samples): (200 uL)(11 tubes)=2200 mL of Qubit working solution (11 uL of Qubit reagent plus 2189 uL of Qubit buffer).
   6.6.5 Add 190 uL of Qubit working solution into each of the 2 standard tubes. Make sure to pre-wet the pipette tip with Qubit working solution before adding the solution to the tubes to prevent introduction of bubbles to the reaction.
   6.6.6 3-20 uL of sample DNA can be used for quantitation with the Qubit dsDNA Broad Range kit with a total volume assay tube volume of 200 uL. Add the necessary volume of Qubit working solution to each assay tube. For example: when 3 uL of sample is used for quantitation, add 197 uL of Qubit working solution to the sample tube. Make sure to pre-wet the pipette tip with Qubit working solution before adding the solution to the tubes.
   6.6.7 Add 10 uL of each standard to the appropriate standard tube. Briefly vortex mix each standard tube.
   6.6.8 Add the necessary amount of sample stock DNA to the appropriate sample tube to yield a total reaction volume of 200 uL. For example: 3 uL of sample DNA to the sample reaction tube containing 197 uL of Qubit working solution. Briefly vortex mix each sample tube.
   6.6.9 Incubate all standard and sample tubes for 2 min at room temperature.
   6.6.10 First calibrate the Qubit 4 Fluorometer using the standards
      6.6.10.1 Tap the screen of the fluorometer to get the instrument out of standby mode.
      6.6.10.2 Select the dsDNA option on the home screen
      6.6.10.3 On the next screen, select dsDNA: Broad range
      6.6.10.4 The fluorometer will prompt to choose between reading new standards and using the previous calibration. Always select Read Standards. Note: Never choose to read samples using the previous calibration (Run samples selection) as this will not yield the most accurate sample concentration.
      6.6.10.5 When prompted, insert Standard #1 sample into the Qubit. Close the lid on the sample chamber. Select Read Standard to read Standard #1.
      6.6.10.6 When prompted, remove Standard #1 from the sample chamber and insert Standard #2. Close the sample chamber lid and select Read standard to read Standard #2.
      6.6.10.7 If the calibration is successful, the Qubit will display the results of the calibration once it is done reading Standard #2. If the calibration fails, the Qubit will display a Calibration error message.
      6.6.10.8 Confirm that the reading given for Standard #2 is at least 10 times greater than that reading given for Standard #1.
      6.6.10.9 If a calibration error is displayed, or if Standard #2 is not 10 times greater than Standard #1. The samples and standards should be made again with fresh Qubit working solution. Do not reuse the tube used to make the previous Qubit working solution. Repeat the calibration using the fresh standards.
         6.6.10.9.1 If the calibration passes and the reading for Standard #2 is at least 10 times greater than that for Standard #1, continue to read the samples (see steps 5.6.9.10-5.6.9.14).
         6.6.10.9.2 If another calibration error is displayed, or if Standard #2 is not 10 times greater than Standard #1, contact the assay SME or management.
      6.6.10.10 Once the calibration has been determined to be successful, select Run samples at the bottom of the Standards results screen to begin to run the samples.
      6.6.10.11 A sample volume screen will be displayed before running the first sample. Use the + or − symbols to select the sample volume used for all of the samples to be run (3-20 uL). Then select the units (ug/uL) for the output of the sample concentration from the dropdown menu.
      6.6.10.12 Once the correct sample volume and sample concentration units are selected, insert sample 1 into the sample chamber. Close the sample chamber lid and select Read tube.
      6.6.10.13 Both the original calculated sample concentration and the concentration of the sample in the Qubit tube are displayed. The original calculated sample concentration is that of the stock DNA sample.
         6.6.10.13.1 If the sample concentration is outside that of the kit range, an Out of Range error will be displayed.
         6.6.10.13.2 Press the right arrow to open the graph of the standards and sample results to determine if the sample was too high or too low.
         6.6.10.13.3 Samples that are out of range should be run again. Use a higher sample volume for sample concentrations that were too low. Use either lower sample volume or a dilution of the stock DNA (made in low EDTA TE buffer) for samples concentrations that were too high. Repeated samples should be run against new standards. Both the samples and standards must be set up using fresh Qubit working solution (do not reuse the tube for the previous Qubit working solution).
      6.6.10.14 Remove sample 1 from the sample chamber. If more than one sample is to be read, insert the next sample into the sample chamber, close the sample chamber lid and select Read tube.
      6.6.10.15 Continue to repeat 5.6.9.14 until all samples are run.
      6.6.10.16 Connect the Qubit to a computer using the USB cable provided with the Qubit fluorometer and when the AutoPlay window opens, select Open device to view files. Continue with step 5.6.9.17 on the Qubit before making any further selections on the computer.
      6.6.10.17 Select Data from the Sample concentration screen for the last sample read or the Home screen to open the Export data screen showing a list of the assays run on the Qubit. Data is listed by assay, showing the Date/Time of the assay, the assay name (dsDNA Broad Range) and the number of sample(s) run in that assay.
         Note: The Qubit 4 Fluorometer saves the data for up to 1000 samples.
      6.6.10.18 Touch the box next to the assay data to be exported. A check mark will appear in the box. Then select Export to export the entire data set to the computer.
      6.6.10.19 On the computer, double click Internal storage, then the Qubit 4 folder to access the exported data.
         Note: The folder will be named QubitData Day-Month-Year with the date being the day the data was exported, not the date the exported data was run.
      6.6.10.20 Open the data folder and save the QubitData_Day-Month-Year_Minute-Hour-Seconds.csv file to a secure data backup system (for example: OpenLab) or per site specific procedures. This file should also be attached to the assay documentation per site specific procedures. Note: The folder will also contain a QubitData_Rna_iq_Day-Month-Year_Minute-Hour-Seconds.csv file that is specific to the RNA IQ assay only.
         This .csv file is empty when running any assay other than RNA IQ and therefore does not need to be saved.
      6.6.10.21 The .csv file contains the results for the assay run. The results will be given in the reverse order in which the samples were read (i.e.: last sample read to first sample read). The Test Date column also indicates the time which each sample was read and can be used to confirm the sample order, with the samples run first having the earlier time stamp vs those run last having the later time stamps.
      6.6.10.22 The Original sample concentration is the concentration of the stock DNA. If a dilution of the stock DNA was run in the Qubit assay, the Original sample concentration will need to be multiplied by the dilution factor of the diluted DNA stock used in the Qubit assay to determine the concentration of the stock DNA. For example: Stock DNA diluted 1:10 in low EDTA TE buffer and 3 uL of the 1:10 dilution used in the Qubit assay, Original sample concentration value from the Qubit is 0.0561 ug/uL, then the undiluted stock DNA concentration is (0.0561 ug/uL)(10) =0.561 ug/uL.
6.7 Diluting Sample DNA in Preparation of Running in the Transgene qPCR Assay:
   6.7.1.1 Samples should be diluted to 0.020 ug/uL in low EDTA TE buffer. Immediately after quantifying the stock DNA. Samples that have stock DNA concentrations less than 0.020 ug/uL should be aliquoted per step 5.7.1.5 and used straight (neat) in the qPCR assay.
   6.7.1.2 Use the following formula to determine the total volume of 0.020 ug/uL diluted DNA that can be made:

$$Total volume of 0.020 u g \text{/} u L diluted DNA = \frac{Volume of stock DNA * Stock DNA concentration}{0.020 u g \text{/} u L}$$

6.7.1.3 Then determine the amount of low EDTA TE buffer needed to dilute the stock DNA to 0.020 ug/uL as follows:

Total volume of 0.02 ug/uL diluted DNA−Volume of Stock DNA=Volume of TE buffer
6.7.1.4 Dilute the desired volume of stock DNA with the calculated volume of low EDTA TE buffer calculated in 5.7.1.3 to make the 0.020 ug/uL working concentration of DNA. It is recommended that as much of the stock DNA is diluted to the qPCR working concentration of 0.020 ug/uL to ensure as many single use aliquots of sample are made as possible. However, a minimum of 3 single use aliquots of 0.020 ug/uL sample DNA is required to be made to ensure enough aliquots for a minimum of 3 qPCR assays.
   For example: Stock DNA concentration of 0.0561 ug/uL. After using 3 uL of DNA in the Qubit assay, minimum of 47 uL of stock DNA remaining. 40 uL of stock DNA will be used to dilute to the qPCR working concentration of

$$Total volume of 0.020 u g \text{/} u L diluted DNA = \frac{40 u L * 0.0561 u g \text{/} u L}{0.020 u g \text{/} u L}$$

Total volume of 0.020 ug/uL diluted DNA=112.2 uL

112.2 uL−40 uL=Volume of TE buffer

Volume of TE buffer=72.2 uL
Dilute 40 uL of stock DNA in 72.2 uL of low EDTA TE buffer to make 112.2 uL total volume of 0.020 ug/uL qPCR working stock sample DNA.
6.7.1.5 Make as many 20 uL single use aliquots of the 0.020 ug/uL working stock sample DNA as possible. 15 uL of DNA is used in each qPCR assay, so each aliquot will have ˜5 uL of excess DNA. It is recommended that a minimum of 3 single use aliquots are made whenever possible.
6.7.1.6 All aliquots should be labeled with a minimum of the sample name, concentration of the aliquot (either 0.020 ug/uL or stock concentration if the stock concentration is less than 0.020 ug/uL) and date aliquots were made.
6.7.1.7 All aliquots as well as any remaining stock sample DNA should be stored at −20° C.

Making and Qualifying New Lots of Oligos

1.0 Purpose
1.1 An example procedure is described for making and qualifying new lots of oligos for the transgene qPCR Method.

2.0 Equipment
2.1 Centrifuge capable of spinning 1.5 mL microcentrifuge tubes (For example: Beckman Coulter, Allegra X-14R with SX4750 rotor and swing set for 96 well plate and adaptors for 1.5 mL microcentrifuge tubes)
2.2 QuantStudio 6 Real-Time PCR System
2.3 Freezer capable of −20° C.
2.4 Calibrated 8 or 12 Channel Pipettes (20, 50 uL or other appropriate size), for example: Rainin pipettes
2.5 Calibrated single Channel Pipettes (20, 100, 200, 1000 uL or other appropriate size), for example: Rainin pipettes
2.6 Refrigerator or cold room capable of maintaining 2-8° C.
2.7 QuantStudio PCR Software v1.3 or greater
2.8 Heat block capable of 55° C. and suitable for 1.5 mL microcentrifuge tubes
2.9 Vortex mixer

3.0 Materials
A Note: Materials designated “for example” may be substituted by similar materials without prior qualification. For materials designated “or equivalent”, alternatives should be demonstrated to be equivalent prior to use for testing samples.
3.1 DNase/RNase-Free Water, for example: Invitrogen Cat #10977015.
3.2 TaqPath ProAmp Master Mix, ThermoFisher Cat #A30866, or equivalent.
3.3 BCMA Transgene and hALB Primers and Probe lyophilized stocks, custom sequences through IDT or equivalent.
3.4 Qualified Lot of BCMA Transgene and ALB Primers and Probe, custom sequences through IDT or equivalent.
3.5 Qualified Lot of BCMA Transgene Standard #1
3.6 Qualified Lot of BCMA Transgene Mid and Low Controls
3.7 1× Low EDTA TE Buffer pH 8.0, RNase/DNase free, for example: Quality Biological Cat #351-324-721.
3.8 0.5 mL Centrifuge tubes, sterile, RNase/DNase free, for example: VWR Screw-Cap Microcentrifuge Tubes Cat #89004-286 or Eppendorf Cat #022431005
3.9 1.5 mL Centrifuge tubes, sterile, RNase/DNase free, for example: Eppendorf Cat #022431021
3.10 2 mL Centrifuge tubes, sterile, RNase/DNase free, for example: Eppendorf Cat #022431048
3.11 5 mL Centrifuge tubes, sterile, RNase/DNase free, for example: Eppendorf Cat #0030119460
3.12 Pipette tips, sterile, filtered, (20, 200, 1000 uL or other appropriate size), for example: Rainin Cat #30389226, 30389240, 30398213
3.13 96 well PCR plates, Applied Biosystems, Cat #4483343, 4483354, 4483349, 4483350, 4483395 or equivalent
3.14 Micro Amp Optical Adhesive Film, Applied Biosystems, Cat #4311971, or equivalent
3.15 Reagent reservoirs, sterile, RNase/DNase free, for example: VistaLabs Cat #3054-1002

4.0 Precautions
4.1 Wear appropriate PPE when working in the laboratory.
4.2 Follow site specific guidelines for working with hazardous chemicals. Consult manufacturer's SDS for more details.
4.3 All pipetting steps must be performed using aseptic technique in a BSC. It is recommended that analysts wear disposable sleeve covers during all steps of the procedure to minimize the risk of contaminating any materials or reagents.

6.0 Procedure
Note: Oligos are qualified as a multiplex set (BCMA transgene and hALB). A lot of oligos is depleted once the last aliquot of any of the of the 6 oligos in the set is used. Do not mix and match oligos from one qualified set to another. Any remaining oligos from a previously qualified set that has been depleted should be discarded.
6.1 Order the BCMA Transgene oligos and hALB oligos (see Table 9 for sequences).

TABLE 9
Transgene qPCR Oligos

| | | Quantity to Order | |
|---|---|---|---|
| Oligo Name | Sequence (5′-3′) | (minimum) | Purification |
| BCMA Transgene | CCA GTA CAA ACT ACT CAA GAG G | 25 nmole DNA Oligo | Standard |
| FORWARD | | | Desalting |
| BCMA Transgene | GCT GAA CTT CAC TCT CAG TT | 25 nmole DNA Oligo | Standard |
| REVERSE | | | Desalting |
| BCMA Transgene | /56-FAM/TC TTC TGG A/ZE/A ATC | 250 nm PrimeTime 5′ | HPLC |
| PROBE | GGC AGC TAC AGC /3IABkFQ/ | 6-FAM/ZEN/3′ IB FQ | |
| hALB FORWARD | TCA TCT CTT GTG GGC TGT AAT C | 25 nmole DNA Oligo | Standard |
| | | | Desalting |
| hALB REVERSE | TGC TGG TTC TCT TTC ACT GAC | 25 nmole DNA Oligo | Standard |
| | | | Desalting |
| hALB PROBE | /5HEX/AG GGA GAG A/ZEN/T TTG | 250 nm PrimeTime 5′ | HPLC |
| | TGT GGG CAT GAC /3IABkFQ/ | 6-HEX/ZEN/3′ IB FQ | |

6.2 Oligos will be supplied lyophilized from the vendor along with specification sheets for each oligo. Store the lyophilized oligos at −20° C. until ready to reconstitute. Lyophilized oligos can be stored at −20° C. for up to 12 months. Note: On average it takes at least 2 weeks to receive the oligos from the manufacturer. Therefore, at least one backup lyophilized set of oligos should be kept at all times to minimize impact to sample testing should an issue arise with either a new or qualified lot of oligos.
6.3 Reconstituting Oligos
   6.3.1 Remove the lyophilized oligo stocks from −20° C. and centrifuge at 10,000×g for 30 sec to ensure all oligo stocks are brought down to the bottom of the tubes before opening the tubes. Do not open the tubes before centrifuging to prevent loss of any oligo that was dislodged from the bottom of the tube during shipping.
   6.3.2 Reconstitute each oligo to 100 uM stocks using low EDTA TE buffer as follows:
      6.3.2.1 Find the nmoles quantity for each oligo on the individual oligo specification sheet.
      6.3.2.2 Multiply the nmoles quantity by 10 in order to get the volume of TE buffer that needs to be added to the oligo tube to generate a 100 uM stock.
      6.3.2.3 Add the volume of TE buffer calculated in step 11.3.2.2 for each oligo to the respective oligo tube.
      6.3.2.4 Briefly vortex each oligo tube to resuspend the lyophilized oligo completely in the TE buffer.
      6.3.2.5 Check the primer tubes to ensure all the lyophilized oligo have been completely dissolved in the TE buffer. If it looks like there may be some particles of oligo that has not been completely dissolved in the TE buffer, the tubes may be heated at 55° C. for 1-5 min to aid in resuspension. After heating, briefly vortex the tubes again.
   6.3.3 Diluting 100 uM Stocks to the Oligo Working Stock Concentrations
      6.3.3.1 Dilute the 100 uM primer stocks to 10 uM working stocks using low EDTA TE buffer
      6.3.3.2 Dilute the 100 uM probe stocks to 10 uM working stocks using low EDTA TE buffer

TABLE 10
Example Dilution of 100 uM Oligo Stock
to Working Stock Concentrations

| | | | Volume of | Volume | | |
|---|---|---|---|---|---|---|
| | Stock | Desired | 100 uM | of TE | Total | |
| Oligo | Conc | Conc | Stock | Buffer | Volume | Dilution |
| Name | (uM) | (uM) | (uL) | (uL) | (uL) | Factor |
| BCMA | 100 | 10 | 120 | 1080 | 1200 | X10 |
| Transgene | | | | | | |
| FORWARD | | | | | | |
| BCMA | 100 | 10 | 120 | 1080 | 1200 | X10 |
| Transgene | | | | | | |
| REVERSE | | | | | | |
| BCMA | 100 | 10 | 120 | 1080 | 1200 | X10 |
| Transgene | | | | | | |
| PROBE | | | | | | |

6.3.3.3 Make 20 uL aliquots of all primers and 35 uL aliquots of all probes in 0.5 mL screw-cap tubes. The aliquot volumes specified are enough for half PCR plate assay. Primers and probes may be aliquoted in larger volumes to support up to full plate assays if needed.
6.3.3.4 Label all aliquots with the minimum information:
   6.3.3.4.1 Oligo Name
   6.3.3.4.2 uM Concentration
   6.3.3.4.3 Lot#
   6.3.3.4.4 Expiry Date (1 year from date of reconstitution)
   6.3.3.4.5 Store all working stocks at −20° C.
6.3.4 Qualifying New Lots of Oligos
   6.3.4.1 Both the currently qualified lot of oligos and the new lot of oligos are run in a minimum of 3 independent transgene qPCR assays using a qualified lot of Standard #1, Mid and Low Controls following protocol as follows:
      6.3.4.1.1 Two master mixes are setup for each of the 3 assays, one master mix will be made using the currently qualified lot of oligos and the second master mix will be made using the new lot of oligos.
      6.3.4.1.2 Load the qPCR plate according to the plate map.
      6.3.4.1.3 Open the “Oligo Qualification.edt” template”, select “Save As”, enter an appropriate name for the qPCR experiment and save as a.eds file. Do not save over the template file.
      6.3.4.1.4 Load and run the qPCR plate according the protocol.
   6.3.4.2 In the Setup section, select Assign. Highlight wells D1-E12, right click and select omit. This will omit the new oligo lot reactions from analysis. Select the entire plate and click Analyze.
   6.3.4.3 Print a PDF report of the data and indicate that it is the analysis for the qualified oligo lot.
      6.3.4.3.1 All assay acceptance criteria described in DSTMD-24448 must be met. If any of the assay acceptance criteria are not met, the assay is invalid and must be repeated. Document any invalid assays in the reagent qualification report.
   6.3.4.4 In the Setup section, select Assign. Highlight wells D1-E12, right click and select include. Highlight wells A1-B12, right click and select omit. This will omit the qualified oligo lot reactions from analysis. Select the entire plate and click Analyze.
   6.3.4.5 Print a PDF report of the data and indicate that it is the analysis for the new oligo lot.
      6.3.4.5.1 All results from the new oligo lot analysis must meet all assay acceptance criteria described herein.
      6.3.4.5.2 Calculate the % Difference in the average Ct values for each standard in the qualified and new oligo lot reactions as follows:

$$\% Difference = ( \frac{\begin{matrix}ABS ( Std \# X Avg Ct Qualified Oligo - \\ Std \# X Avg Ct New Oligo )\end{matrix}}{\begin{matrix}Avg ( Std \# X Avg Ct Qualified Oligo \& \\ Std \# X Avg Ct New Oligo )\end{matrix}} ) * 100$$

6.3.4.5.3 All % Differences must be ≤1.60%
6.3.4.6 All valid qualification assays must meet the Ct % Difference criteria in order for the new oligo lot to pass qualification.
6.3.4.7 If the new lot does not meet the acceptance criteria, the lot fails qualification and must be discarded. Prepare another new lot of oligos and execute the qualification on the new lot.

Making Working Linear LiCAR Plasmid Stocks

1.0 Purpose
1.1 This example describes an example procedure for linearizing the LiCAR plasmid and converting mg/mL plasmid concentrations to copies/uL in order to make working stocks of the linear plasmid for use in making standard and controls for the transgene qPCR Method.

2.0 Equipment
2.1 Capable of spinning 1.5 mL and 5 mL microcentrifuge tubes (For example: Beckman Coulter, Allegra X-14R with SX4750 rotor with adaptors for 1.5 mL microcentrifuge tubes).
2.2 Electrophoresis apparatus, for example Lonza FlashGel DNA System and FlashGel Dock or Invitrogen E-Gel Electrophoresis Device
2.3 Freezer capable of −70° C.
2.4 Refrigerator or cold room capable of maintaining 2-8° C.
2.5 Calibrated single Channel Pipettes (20, 100, 200, 1000 uL or other appropriate size), for example: Rainin pipettes
2.6 Qubit 4 Fluorometer, Invitrogen Cat #Q33226
2.7 Heat block capable of 37° C. and suitable for 1.5 mL microcentrifuge tubes
2.8 Biosafety Cabinet
2.9 Vortex mixer

3.0 Materials
Note: Materials designated “for example” may be substituted by similar materials without prior qualification. For materials designated “or equivalent”, alternatives must be demonstrated to be equivalent prior to use for testing samples.
3.1 pLLV-LICAR2SIN LiCAR plasmid stock
3.2 DNase/RNase-Free Water, for example: Invitrogen Cat #10977015
3.3 EcoRI HF Enzyme, New England Biolabs Cat #R3101S, or equivalent
3.4 GeneJet Gel Extraction & DNA Cleanup Kit, ThermoFisher, Cat #K0831, or equivalent
3.5 Pre-cast electrophoresis gels, for example Lonza FlashGel DNA Cassette or Invitrogen E-Gels suitable for high molecular weight band resolution (for example: FlashGel DNA Cassettes, 1.2% 12+1 single-tier, Lonza Cat #57023)
3.6 Molecular weight DNA ladder suitable for determination of at least 8000 kb band size (for example: E-Gel 1 Kb Plus DNA Ladder, Invitrogen Cat #10488090) 1.1 1.5 mL Centrifuge tubes, sterile, RNase/DNase free, for example: Eppendorf Cat #022431021 1.2 2 mL Centrifuge tubes, sterile, RNase/DNase free, for example: Eppendorf Cat #022431048
3.7 5 mL Centrifuge tubes, sterile, RNase/DNase free, for example: Eppendorf Cat #0030119460
3.8 1× Low EDTA TE Buffer pH 8.0, RNase/DNase free, for example: Quality Biological Cat #351-324-721.
3.9 0.5 mL Centrifuge tubes, sterile, RNase/DNase free, for example: VWR Screw-Cap Microcentrifuge Tubes Cat #89004-286 or Eppendorf Cat #022431005
3.10 Pipette tips, sterile, filtered, (20, 200, 1000 uL or other appropriate size), for example: Rainin Cat #30389226, 30389240, 30398213

4.0 Precautions
4.1 Wear appropriate PPE when working in the laboratory.
4.2 Follow site specific guidelines for working with hazardous chemicals. Consult manufacturer's SDS for more details.
4.3 All pipetting steps must be performed using aseptic technique in a BSC. It is recommended that analysts wear disposable sleeve covers during all steps of the procedure to minimize the risk of contaminating any materials or reagents.

5.0 Procedure
5.1 Pre-warm the heat block to 37° C. before beginning plasmid digestion.
5.2 Thaw a vial of LiCAR plasmid stock.
5.3 Determine the concentration of LiCAR DNA using the Qubit 4 Fluorometer and the procedure described previously herein. The plasmid stock may need to be diluted in order to be within the range of the Qubit dsDNA BR kit (2-1000ng of DNA)
5.4 Dilute the necessary amount of plasmid needed to digest 0.09 ug/uL of plasmid DNA in water.
   For example: Plasmid stock concentration of 1.24 ug/uL is diluted to 0.09 ug/uL by adding 3.63 uL of plasmid DNA into 46.37 uL of DNase/RNase-Free water.
5.5 Prepare the EcoRI HF enzyme digestion reaction mixture at room temperature and add each reagent in the order indicated in Table 11.

TABLE 11
Protocol of EcoRI HF Digestion of LiCar Plasmid

| Reaction Component | Volume (uL) |
|---|---|
| 0.09 ug/uL Plasmid DNA | 10 |
| 10X CutSmart Buffer | 5 |
| DNase/RNase Free Water | 34 |
| EcoRI-HF (20,000 U/mL) | 1 |
| Total Volume | 50 uL |

Note:
Multiple 50 uL enzyme digestion reactions can be carried out in replicates, if needed, to yield a sufficient amount of linearized DNA for preparation of a working stock of LiCAR plasmid to make standards and controls.

5.6 Reserve at least 5 uL of undigested plasmid DNA to serve as an intact plasmid DNA control on a gel.
5.7 Gently mix digestion tubes by pipette mixing or flicking the tube (Do not vortex the reaction). Briefly spin down the reaction for 5 seconds at 300×g.
5.8 Incubate tubes in a 37° C. heat block for 15 minutes.
5.9 Purify the digested plasmid DNA using the GeneJet Gel Extraction and DNA clean-up Mini Kit.
   Note: The volume used for DNA purification should not exceed 200 uL and the total amount of plasmid DNA purified should not exceed 10 ug. If the total volume exceeds 200 uL, divide the volume equally into 2 or more tubes before proceeding with the following purification steps.
5.10 When opening a new GenJet kit, prior to starting the DNA cleanup procedure, add 96-100% Ethanol to the Wash Buffer bottles according to the instructions on each bottle. Confirm addition of ethanol by marking the date added and initials on the label. Store wash buffers with ethanol at RT. Also check all solutions in the kit for any salt precipitation before use. Dissolve any precipitates by warming the solutions to 37° C. and then equilibrating to RT before use.
   Note: DNA purification columns are to be stored at 2-8° C. upon kit arrival and when columns are not in use. Be sure to close the bag with the DNA purification columns tightly after each use.
5.11 Adjust the volume of the reaction mixture to 200 uL with DNase/RNase Free water. For Example: add 150 uL water to the 50 uL digested DNA mixture.
5.12 Add 100 μL of Binding Buffer. Mix thoroughly by pipetting.
5.13 Add 300 μL of ethanol (96-100%) and mix by pipetting.
5.14 Transfer the mixture to a DNA Purification Micro Column and collection tube. Centrifuge the column for 1 minute at 14,000×g. Discard the flow-through. Place the DNA Purification Micro Column back into the collection tube. Alternatively, the tube may be placed in a 2 mL microcentrifuge tube and the collection tube discarded.
5.15 Add 700 μL of Wash Buffer (supplemented with ethanol) to the column and centrifuge for 1 minute at 14,000×g. Discard the flow-through and place the column back into the collection tube. Alternatively, the tube may be placed in a 2 mL microcentrifuge tube and the collection tube discarded.
5.16 Perform another wash step by adding 700 μL of Wash Buffer to the column and centrifuge for 1 minute at 14,000×g. Discard the flow-through and place the column back into the same collection tube. Alternatively, the tube may be placed in a 2 mL microcentrifuge tube and the collection tube discarded.
5.17 Centrifuge the column for an additional 1 minute at 14,000×g to completely remove any residual wash buffer.
5.18 Transfer the column to a clean 1.5 mL microcentrifuge tube and add 10 uL of Elution Buffer (supplied with the GeneJet kit) to the center of the column. Do not touch or pierce the silica membrane with the pipette tip.
5.19 Centrifuge for 1 minute at 14,000×g to elute the DNA.
5.20 Confirm digestion of the VSV-G plasmid by gel electrophoresis of intact circular (undigested DNA) and linearized DNA. It is recommended to make a dilution of the linearized plasmid stock to use for gel electrophoresis to conserve the maximum amount of linearized plasmid possible.
5.21 Determine the concentration of purified linear VSV-G plasmid DNA using the Qubit 4 Fluorometer and the procedure described previously herein.
5.22 Convert the linear plasmid concentration determined in step 5.21 to copy number/uL of LiCAR plasmid as follows:

$$( plasmid conc . from Qubit ( u g \text{/} u L ) ) ( \frac{1 g}{1 \times {10}^{6} u g} ) = plasmid conc . ( g \text{/} u L )$$

$$\frac{\begin{matrix}( 6.023 \times {10}^{23} copies \text{/} mol ) \\ ( plsmid conc . ( g \text{/} u L ) )\end{matrix}}{( 660 g \text{/} mol ) ( plasmid size ( bp ) )} = plasmid conc . ( copies \text{/} u L )$$

For example: LiCAR DNA concentration is 1.03 ug/uL.

$$( 1.03 u g \text{/} u L ) ( \frac{1 g}{1 \times {10}^{6} u g} ) = 1.03 \times {10}^{- 6} g \text{/} u L$$

$$\frac{( 6.023 \times 10.23 copies \text{/} mol ) ( 1.03 \times {10}^{- 6} g \text{/} u L )}{( 660 g \text{/} mol ) ( 8518 bp )} = 1.103 \times {10}^{11} copies / u L$$

5.23 Dilute the plasmid stock to a working concentration appropriate for making standard and controls in low EDTA TE buffer.
5.24 Dilute the linear LiCAR plasmid stock in low EDTA TE buffer as necessary to make a working stock linear LiCAR plasmid appropriate for making standards and controls.
5.25 Make appropriate-sized single use aliquots of the working plasmid concentration labelled with the minimum information:
   5.25.1 Linear Plasmid
   5.25.2 Plasmid concentration in copies/uL
   5.25.3 Aliquot size
   5.25.4 Expiration date
5.26 Plasmids are stable for 12 months at −70° C.

Making and Qualifying New Lots of Standard

1.0 Purpose
1.1 An example procedure for making and qualifying new lots of standard for the transgene qPCR method is described.

2.0 Equipment
2.1 Centrifuge capable of spinning 1.5 mL microcentrifuge tubes (For example: Beckman Coulter, Allegra X-14R with SX4750 rotor and swing set for 96 well plate and adaptors for 1.5 mL microcentrifuge tubes)
2.2 QuantStudio 6 Real-Time PCR System
2.3 Freezer capable of −20° C.
2.4 Freezer capable of −70° C.
2.5 Calibrated 8 or 12 Channel Pipettes (20, 50 uL or other appropriate size), for example: Rainin pipettes
2.6 Calibrated single Channel Pipettes (20, 100, 200, 1000 uL or other appropriate size), for example: Rainin pipettes
2.7 Refrigerator or cold room capable of maintaining 2-8° C.
2.8 QuantStudio PCR Software v1.3 or greater
2.9 Heat block capable of 55° C. and suitable for 1.5 mL microcentrifuge tubes
2.10 Vortex mixer
2.11 Biosafety Cabinet

3.0 Materials

Note: Materials designated “for example” may be substituted by similar materials without prior qualification. For materials designated “or equivalent”, alternatives should be demonstrated to be equivalent prior to use for testing samples.
3.1 DNase/RNase-Free Water, for example: Invitrogen Cat #10977015.
3.2 TaqPath ProAmp Master Mix, ThermoFisher Cat #A30866, or equivalent.
3.3 Mock T-cell pellets with 2×106-4×106 cells per pellet.
3.4 Qualified Lot of BCMA Transgene and ALB Primers and Probe, custom sequences through IDT or equivalent.
3.5 Qualified Lot of BCMA Transgene Standard #1
3.6 Qualified Lot of BCMA Transgene Mid and Low Controls
3.7 Working Stock Aliquot of pLLV-LICAR2SIN
3.8 1× Low EDTA TE Buffer pH 8.0, RNase/DNase free, for example: Quality Biological Cat #351-324-721.
3.9 0.5 mL Centrifuge tubes, sterile, RNase/DNase free, for example: VWR Screw-Cap Microcentrifuge Tubes Cat #89004-286 or Eppendorf Cat #022431005
3.10 1.5 mL Centrifuge tubes, sterile, RNase/DNase free, for example: Eppendorf Cat #022431021
3.11 2 mL Centrifuge tubes, sterile, RNase/DNase free, for example: Eppendorf Cat #022431048
3.12 5 mL Centrifuge tubes, sterile, RNase/DNase free, for example: Eppendorf Cat #0030119460
3.13 Pipette tips, sterile, filtered, (20, 200, 1000 uL or other appropriate size), for example: Rainin Cat #30389226, 30389240, 30398213
3.14 96 well PCR plates, Applied Biosystems, Cat #4483343, 4483354, 4483349, 4483350, 4483395 or equivalent
3.15 Micro Amp Optical Adhesive Film, Applied Biosystems, Cat #4311971, or equivalent
3.16 Reagent reservoirs, sterile, RNase/DNase free, for example: VistaLabs Cat #3054-1002

4.0 Precautions
4.1 Wear appropriate PPE when working in the laboratory.
4.2 Follow site specific guidelines for working with hazardous chemicals. Consult manufacturer's SDS for more details.
4.3 All pipetting steps must be performed using aseptic technique in a BSC. It is recommended that analysts wear disposable sleeve covers during all steps of the procedure to minimize the risk of contaminating any materials or reagents.

5.0 Procedure
5.1 Isolated gDNA from mock T-cell pellets following the protocol previously described herein. Mock T-cells should be representative of the CAR T manufacturing process but without lentivector transduction.
5.2 Several silica DNA extraction columns will be used to isolate the needed quantity of gDNA. Combine the eluted DNA from all columns to make a single stock of mock T-cell gDNA before quantification.
5.3 After quantification, make sure enough gDNA is isolated to make the number of aliquots sufficient to support at least 4 months of testing.
   5.3.1 If the initial gDNA isolation does not yield enough gDNA to make a large enough lot to last at least 4 months, additional mock T-cell pellets can be isolated, combined with the initial mock T-cell gDNA stock and the combined stock quantitated using the Qubit.
5.4 Standard #1 is made up of mock T-cell DNA at a concentration of 0.05 ug/uL spiked with pLLV-LICAR2SIN plasmid (LiCAR plasmid) so that 5 uL of Standard #1 contains 121,212.1212 copies of LiCAR plasmid.
   5.4.1 Determine the total volume of Standard #1 that can be made from the stock of mock T-cell DNA as follows:

$$\frac{( Volume of gDNA ) ( Concentration of gDNA )}{0.05 u g \text{/} u L} = Total volume of Standard \#1$$

For Example: Approximately 397.0 uL of mock T-cell gDNA remains after DNA quantification. Concentration of gDNA is 0.0853 ug/uL. 392.0 uL of gDNA will be taken to make Standard #1.

$$\frac{( 392 u L ) ( 0.0853 u g \text{/} u L )}{0.05 u g \text{/} u L} = 668.8 u L Total volume of Standard \#1$$

5.4.2 Then determine the volume of low EDTA TE buffer+plasmid is needed to dilute the gDNA to 0.05 ug/uL as follows:

Total volume of Standard #1−Volume of gDNA=Volume of TE+plasmid

For Example: 392.0 uL of gDNA stock gDNA will be taken to make a total volume of 668.8 uL of Standard #1.

668.8 uL−392.0 uL=276.8 uL of TE+plasmid
5.4.3 Then determine the volume of just plasmid needed to achieve 121,212.121 copies of LiCAR plasmid per 5 uL of Standard #1 as follows:

$$\frac{121 , 212 , 121 copies}{5 u L} = 24 , 242 .4242 copies \text{/} u L$$

$$\frac{( 24 , 242.4242 copies \text{/} u L ) ( Total volume of Standard \#1 )}{Working Plasmid Stock Concentration ( copies \text{/} u L )} = Volume of plasmid$$

For Example: A total volume of 668.8 uL of Standard #1. LiCAR plasmid working stock of 1.1035×106 copies/uL.

$$\frac{( 24 , 242.4242 copies \text{/} u L ) ( 668.8 u L )}{1.1035 \times {10}^{6} ( copies \text{/} u L )} = 14.7 u L of plasmid$$

5.4.4 Finally, determine the volume of just low EDTA TE buffer needed to make the total volume of Standard #1.

Total volume of Standard #1−Volume of gDNA+Volume of plasmid=Volume of TE

For Example: A total volume of 668.8 uL of Standard #1 from 392.0 uL of mock T-cell gDNA and 14.7 uL of LiCAR plasmid.

668.8 uL−(392.0 uL+14.7 uL)=262.1 uL of TE
5.5 Begin to make Standard #1 by transferring the volume of stock mock T-cell gDNA to an appropriate-sized DNase/RNase free tube to accommodate the total volume of Standard #1 to be made (calculated in step 5.4.1)
5.6 To the tube from step 5.5, add the volume of low EDTA TE buffer calculated in step 5.4.4.
5.7 Then add the volume of LiCAR plasmid calculated in step 5.4.3. Briefly vortex, mix the tube.
5.8 Make 20 uL single use aliquots of Standard #1 labeled with the minimum information:
   5.8.1 BCMA Transgene Standard #1
   5.8.2 Lot #
   5.8.3 Date Standard #1 was made
5.9 Store all single use aliquots at −20 oC.
   5.9.1 Qualifying New Lots of Standard #
      5.9.1.1 Both the currently qualified Standard #1 and the new lot of Standard #1 are run in a minimum of 3 independent transgene qPCR assays using a qualified lot of oligos, Mid and Low Controls following protocol previously described herein as follows:
         5.9.1.1.1 One master mix is made for all standard and control samples
         5.9.1.1.2 Two 5-point standard curves are made, one using the qualified lot of Standard #1 and one using the new lot of Standard #1.
         5.9.1.1.3 Load the qPCR plate according to the plate map.
         5.9.1.1.4 Open the “Standard Qualification.edt” template”, select “Save As”, enter an appropriate name for the qPCR experiment and save as a.eds file. Do not save over the template file.
         5.9.1.1.5 Load and run the qPCR plate according to protocol described herein.
      5.9.1.2 In the Setup section, select Assign. Highlight wells C1-D3, right click and select omit. This will omit the new Standard #1 lot standard curve from analysis. Select the entire plate and click Analyze.
      5.9.1.3 Print a PDF report of the data and indicate that it is the analysis for the qualified Standard #1 lot.
         5.9.1.3.1 All assay acceptance criteria described herein must be met. If the any of the assay acceptance criteria are not met, the assay is invalid and must be repeated. Document any invalid assays in the reagent qualification report.
      5.9.1.4 In the Setup section, select Assign. Highlight wells C1-D3, right click and select include. Highlight wells A1-B3, right click and select omit. This will omit the qualified Standard #1 lot standard curve from analysis. Select the entire plate and click Analyze.
      5.9.1.5 Print a PDF report of the data and indicate that it is the analysis for the new Standard #1 lot.
         5.9.1.5.1 All results from the new Standard #1 lot analysis must meet all assay acceptance criteria described herein.
      5.9.1.6 The average of all triplicate VCN/cell results for the Mid and Low controls across all valid qualification assays must be ≤20% of the expected VCN/cell results for the new lot of Standard #1 to pass qualification.

Making and Qualifying New Lots of Mid and Low Controls

1.0 Purpose
1.1 An example procedure for making and qualifying new lots of standard for the Transgene qPCR Method is described.

2.0 Equipment
2.1 Centrifuge capable of spinning 1.5 mL microcentrifuge tubes (For example: Beckman Coulter, Allegra X-14R with SX4750 rotor and swing set for 96 well plate and adaptors for 1.5 mL microcentrifuge tubes)
2.2 QuantStudio 6 Real-Time PCR System
2.3 Freezer capable of −20° C.
2.4 Freezer capable of −70° C.
2.5 Calibrated 8 or 12 Channel Pipettes (20, 50 uL or other appropriate size), for example: Rainin pipettes
2.6 Calibrated single Channel Pipettes (20, 100, 200, 1000 uL or other appropriate size), for example: Rainin pipettes
2.7 Refrigerator or cold room capable of maintaining 2-8° C.
2.8 QuantStudio PCR Software v1.3 or greater
2.9 Heat block capable of 55° C. and suitable for 1.5 mL microcentrifuge tubes
2.10 Vortex mixer
2.11 Biosafety Cabinet

3.0 Materials

Note: Materials designated “for example” may be substituted by similar materials without prior qualification. For materials designated “or equivalent”, alternatives should be demonstrated to be equivalent prior to use for testing samples.
3.1 DNase/RNase-Free Water, for example: Invitrogen Cat #10977015.
3.2 TaqPath ProAmp Master Mix, ThermoFisher Cat #A30866, or equivalent.
3.3 Mock T-cell pellets with 2×106-4×10⁶ cells per pellet.
3.4 Qualified Lot of BCMA Transgene and ALB Primers and Probe, custom sequences through IDT or equivalent.
3.5 Qualified Lot of BCMA Transgene Standard #1
3.6 Qualified Lot of BCMA Transgene Mid and Low Controls
3.7 Working Stock Aliquot of pLLV-LICAR2SIN
3.8 1× Low EDTA TE Buffer pH 8.0, RNase/DNase free, for example: Quality Biological Cat #351-324-721.
3.9 0.5 mL Centrifuge tubes, sterile, RNase/DNase free, for example: VWR Screw-Cap Microcentrifuge Tubes Cat #89004-286 or Eppendorf Cat #022431005
3.10 1.5 mL Centrifuge tubes, sterile, RNase/DNase free, for example: Eppendorf Cat #022431021
3.11 2 mL Centrifuge tubes, sterile, RNase/DNase free, for example: Eppendorf Cat #022431048
3.12 5 mL Centrifuge tubes, sterile, RNase/DNase free, for example: Eppendorf Cat #0030119460
3.13 Pipette tips, sterile, filtered, (20, 200, 1000 uL or other appropriate size), for example: Rainin Cat #30389226, 30389240, 30398213
3.14 96 well PCR plates, Applied Biosystems, Cat #4483343, 4483354, 4483349, 4483350, 4483395 or equivalent
3.15 Micro Amp Optical Adhesive Film, Applied Biosystems, Cat #4311971, or equivalent
3.16 Reagent reservoirs, sterile, RNase/DNase free, for example: VistaLabs Cat #3054-1002

4.0 Precautions
4.1 Wear appropriate PPE when working in the laboratory.
4.2 Follow site specific guidelines for working with hazardous chemicals. Consult manufacturer's SDS for more details.
4.3 All pipetting steps must be performed using aseptic technique in a BSC. It is recommended that analysts wear disposable sleeve covers during all steps of the procedure to minimize the risk of contaminating any materials or reagents.

5.0 Procedure
5.1 Isolated gDNA from mock T-cell pellets following the protocol in protocol previously described herein. Mock T-cells should be representative of the CAR T manufacturing process but without lentivector transduction.
5.2 Several silica DNA extraction columns will be used to isolate the needed quantity of gDNA. Combine the eluted DNA from all columns to make a single stock of mock T-cell gDNA before quantification.
5.3 After quantification, make sure enough gDNA is isolated to make the number of aliquots sufficient to support at least 4 months of testing. The Low Control is a 1:10 dilution of the Mid Control using 0.02 ug/uL mock T-cell gDNA as the diluent. The Low and Mid Controls do not need to be made together from the same mock T-cell gDNA stock, but if they are made together, a portion of the isolated mock T-cell DNA stock will need to be preserved to make the Low Control. The example shown in the steps below is an example of how to make a Mid and Low Control with the same stock of mock T-cell gDNA.
   5.3.1 If the initial gDNA isolation does not yield enough gDNA to make a large enough lot of both controls to last at least 4 months, additional mock T-cell pellets can be isolated, combined with the initial mock T-cell gDNA stock and the combined stock quantitated using the Qubit.
5.4 BCMA Transgene Mid Control is made up of mock T-cell DNA at a concentration of 0.02 ug/uL spiked with pLLV-LICAR2SIN plasmid (also referenced herein as “LiCAR” plasmid) so that 5 uL of the Mid Control contains 30,303.030 copies of LiCAR plasmid. This equates to a vector copy number of 2.00 copies/cell in 5 uL of the control.
   5.4.1 Determine the total volume of Mid Control that can be made from the stock of mock T-cell DNA as follows:

$$\frac{( Volume of gDNA ) ( Concentration of gDNA )}{0.02 u g \text{/} u L} = Total volume of Mid Control$$

For Example: Approximately 547.0 uL of mock T-cell gDNA remains after DNA quantification. Concentration of gDNA is 0.0913 ug/uL. 298 uL of gDNA will be taken to make the Mid Control.

$$\frac{( 298.0 u L ) ( 0.0913 u g \text{/} u L )}{0.02 u g \text{/} u L} = 1360.4 u L Total volume of Mid Control$$

5.4.2 Then determine the volume of low EDTA TE buffer+plasmid is needed to dilute the gDNA to 0.02 ug/uL as follows:

Total volume of Mid Control−Volume of gDNA=Volume of TE+plasmid

For Example: 298.0 uL of gDNA stock gDNA will be taken to make a total volume of 1360.4 uL of Mid Control.

1360.4 uL−298.0 uL=1062.4 uL of TE+plasmid
5.4.3 Then determine the volume of just plasmid needed to achieve 30,303.0303 copies of LiCAR plasmid per 5 uL of Mid Control as follows:

$$\frac{30 , 303.030 copies}{5 u L} = 6 , 060.606 copies \text{/} u L$$

$$\frac{( 6 , 060.606 copies \text{/} u L ) ( Total volume of Mid Control )}{Working Plasmid Stock Concentration ( copies \text{/} u L )} = Volume of plasmid$$

For Example: A total volume of 1360.4 uL of Mid Control. LiCAR plasmid working stock of 1.1035×106 copies/uL.

$$\frac{( 6 , 060.606 \; 1 copies \text{/} u L ) ( 1360.4 uL )}{1.1035 \times {10}^{6} ( copies \text{/} u L )} = 7.5 u L of plasmid$$

5.4.4 Finally, determine the volume of just low EDTA TE buffer needed to make the total volume of Mid Control.

Total volume of Mid Control−Volume of gDNA+Volume of plasmid=Volume of TE

For Example: A total volume of 1360.4 uL of Mid Control from 298 uL of mock T-cell gDNA and 7.5 uL of LiCAR plasmid.

1360.4 uL−(298 uL+7.5 uL)=1054.9 uL of TE
5.5 Make the Mid Control by transferring the volume of stock mock T-cell gDNA to an appropriate sized DNase/RNase free tube to accommodate the total volume of Mid Control to be made (calculated in step 5.4.1).
5.6 To the tube from step 5.5, add the volume of low EDTA TE buffer calculated in step 5.4.4.
5.7 Then add the volume of LiCAR plasmid calculated in step 5.4.3. Briefly vortex mix the tube.
5.8 Make Low Control from a Mid Control stock by diluting the Mid Control 1:10 using 0.02 ug/uL mock T-cell gDNA as the diluent. For example: Make 1230.0 uL total volume of Low Control from the Mid Control stock.

$$\frac{1230.0 u L Low Control}{10} = 123.0 u L of Mid Control$$

As the example is to show how to make a Low and Mid Control from one stock of mock T-cell gDNA, the remaining volume of Mid Control will be used as the new lot of Mid Control.

1360.4 uL Mid Control−123.0 uL(to make Low Control)=1237.4 uL of Mid Control remaining
5.9 Determine the amount of 0.02 ug/uL mock T-cell gDNA needed to make the desired total volume of Low Control. For example: Total volume of Low Control stock to be made is 1230.0 uL by diluting the Mid Control 1:10 (123.0 uL Mid Control).

1230.0 uL−123.0 uL=1107.0 uL of 0.02 ug/uL gDNA
5.10 Then dilute the necessary volume of isolated mock T-cell gDNA to 0.02 ug/uL using low EDTA TE buffer. Make enough volume of 0.02 ug/uL gDNA (include overage) needed to make the Low Control (step 5.9). For example: 1107.0 uL of 0.02 ug/uL mock T-cell gDNA needed to make 1230.0 uL of the Low Control. Dilute 0.0913 ug/uL mock T-cell stock to 0.02 ug/uL.

$$\frac{( 244.0 u L gDNA stock ) ( 0.0913 u g \text{/} u L )}{0.02 u g \text{/} u L} = 1113.9 u L total volume 0.02 u g \text{/} u L gDNA$$

$$1113.9 u L - 244.0 u L = 869.9 u L volume TE buffer$$

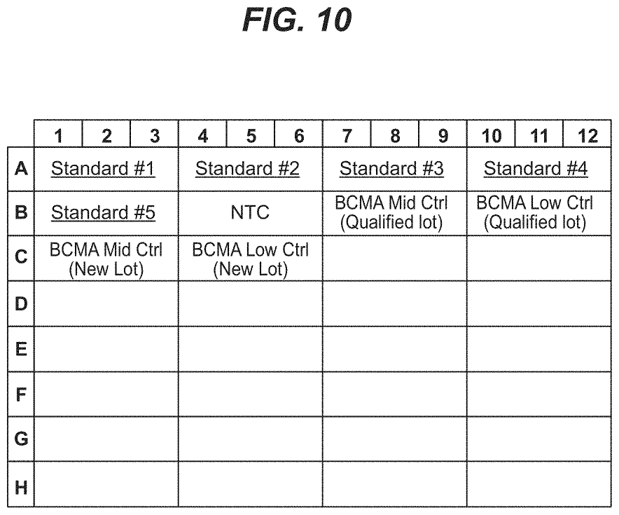

Dilute 244.0 uL of stock mock T-cell gDNA at a concentration of 0.0913 ug/uL with 869.9 uL of low EDTA TE buffer to make enough volume of 0.02 ug/uL mock Tcell gDNA to make 1230.0 uL of the Low Control.
5.11 Make the Low Control by transferring the volume of stock mock T-cell gDNA to an appropriate-sized DNase/RNase free tube to accommodate the desired total volume of Low Control to be made.
5.12 To the tube from step 5.11, add the volume of low EDTA TE buffer calculated in step 5.10.
5.13 Then add the volume of Mid Control needed to make the Low Control. Briefly vortex mix the tube.
5.14 Make 20 uL single use aliquots of Mid and Low Control labelled with the minimum information:
   5.14.1 BCMA Transgene Mid/Low Control
   5.14.2 Lot #
   5.14.3 Date Control was made
5.15 Store all single use aliquots at −20° C.
   5.15.1 Qualifying New Lots of Controls
      5.15.1.1 Both the currently qualified Mid and Low Controls and the new lot of Controls are run in a minimum of 3 independent transgene qPCR assays using a qualified lot of oligos and Standard #1 following protocol previously described herein as follows:
         5.15.1.1.1 One master mix is made for all standard and control samples
         5.15.1.1.2 One 5-point standard curve is made using the qualified lot of Standard #1.
         5.15.1.1.3 Load the qPCR plate according to the plate map in FIG. 10.
         5.15.1.1.4 Open the Controls Qualification template file, select “Save As”, enter an appropriate name for the qPCR experiment and save as a .eds file. Do not save over the template file.
         5.15.1.1.5 Load and run the qPCR plate according Controls Qualification template file.
      5.15.1.2 Analyze the data according to Controls Qualification template file with the new lot of controls analyzed as samples.
      5.15.1.2.1 All assay acceptance criteria described in Controls Qualification template file must be met.
      5.15.1.3 Qualification Acceptance Criteria
         5.15.1.3.1 The average of all triplicate VCN/cell results for the new lot of Mid and Low controls across all valid qualification assays must be ≤35% of the expected VCN/cell results for the new lot of controls to pass qualification.
         5.15.1.3.2 The % CV of all triplicate VCN/cell results for the new lot of Mid and Low controls across all valid qualification assays must be ≤20%.

### EXAMPLE 3

### Transgene qPCR Method Qualification

1.0 Purpose
1.1 The purpose of this example is to describe the results obtained during execution of the transgene qPCR method qualification protocol.

2.0 Scope
2.1 The qPCR assay for quantitation of the LiCAR plasmid integrated into CAR T product was qualified by examining the following qualification parameters: specificity, accuracy, linearity, precision (repeatability and intermediate precision), range and LOQ.

3.0 Definitions and Abbreviations
3.1 qPCR Quantitative Polymerase Chain Reaction
3.2 hALB Human Albumin
3.3 VCN Vector Copy Number
3.4 CV Coefficient of Variation
3.5 SD Standard Deviation
3.6 Ct Cycle Threshold
3.7 LOQ Limit of Quantitation
3.8 BMD Bioassay Methods Development
3.9 QC Quality Control

4.0 Study Approach
4.1 Assay linearity was qualified by testing a 5-point standard curve made by making 5-fold serial dilutions of the transgene qPCR Standard #1 and plotting the results of the standard curve as Log 10 Quantity vs Ct.
4.2 Assay precision, both repeatability and intermediate precision, was qualified by testing the 5-point standard curve as well as the Mid and Low assay controls.
4.3 Assay accuracy was qualified by testing the Mid and Low assay controls.
4.4 Assay specificity was demonstrated by testing mock T-cell DNA sample and a representative CAR T day 10 harvest DNA sample.
4.5 Assay LOQ was determined by testing 0.02 VCN/cell and 0.014 VCN/cell LOQ samples.
4.6 Three qualification assays were performed by two analysts with one of the three assays run on a separate day.

5.0 Materials
5.1 Refer to the qualification protocol described herein for a full list of materials required to perform the transgene qPCR assay.
5.2 Assay Standard and Controls:
   5.2.1 BCMA Transgene Standard #1, Lot #LM-RP3-00533A.
   5.2.2 BCMA Transgene Mid Control, Lot #LM-RP3-00533B.
   5.2.3 BCMA Transgene Low Control, Lot #LM-RP3-00533C.
5.3 Assay Specificity Samples:
   5.3.1 Mock T-cell DNA, Lot #LM-RP3-00533.
   5.3.2 CART DNA, LM-RP3-00541.
5.4 Assay LOQ Samples:
   5.4.1 0.02 VCN/cell and 0.014 VCN/cell LOQ Samples, Lot #LM-RP3-00533.
   5.4.2 Transgene Method Oligo Set Lot #LM-RP3-00460

6.0 Summary
6.1 This example outlines the results obtained during the execution of the transgene qPCR method qualification protocol.
6.2 The method demonstrated acceptable accuracy, precision, specificity and linearity as summarized in Table 12 (10.2-10.5). In addition, the assay range and LOQ were defined for both the transgene and hALB targets. The method is therefore qualified for testing of the CAR T drug product material prior to formulation with cryopreservation media.

TABLE 12
Summary of Acceptance Criteria and Results for the Qualification
of the Transgene Multiplexed qPCR Procedure

| Parameter | Acceptance Criteria | Results |
|---|---|---|
| Linearity | The R² of the linear regression | 1.00 |
| (Transgene | of the Log₁₀ vs Ct values for the | |
| Target) | standard curve across all valid | |
| | qualification assays must be ≥0.97. | |
| Repeatability | The % CV of the triplicate Ct | 0.06-0.54% |
| (Transgene Target | values within each valid | |
| Standard Curve) | qualification assay for each | |
| | standard must be ≤30%. | |
| Intermediate | The % CV of the Ct values | 0.26-0.47% |
| Precision | across all valid qualification | |
| (Transgene Target | assays for each standard must be ≤30%. | |
| Standard Curve) | | |
| Linearity | The R² of the linear regression | 1.00 |
| (hALB Target) | of the Log₁₀ vs Ct values for the | |
| | standard curve across all valid | |
| | qualification assays must be ≥0.97. | |
| Repeatability | The % CV of the triplicate Ct | 0.03-0.41% |
| (hALB Target | values within each valid | |
| Standard Curve) | qualification assay for each | |
| | standard must be ≤30%. | |
| Intermediate | The % CV of the Ct values | 0.17-0.55% |
| Precision | across all valid qualification | |
| (hALB Target | assays for each standard must be ≤30%. | |
| Standard Curve) | | |
| Accuracy | The % recovery for the mid and | Mid Control (2.00 |
| | low assay controls in each valid | VCN/cell): 93-95% |
| | qualification assay must be | recovery |
| | within 70-130% of the expected | Low Control (0.02 |
| | VCN/cell value for that control. | VCN/cell): 79-84% |
| | | recovery |
| Repeatability | The % CV of the triplicate VCN/cell results | Mid Control (2.00 |
| | for the mid and low assay controls within | VCN/cell): 4-6% |
| | each valid qualification assay must be ≤30%. | Low Control (0.02 |
| | | VCN/cell): 4-6%. |
| Intermediate | The % CV of the VCN/cell results for the | Mid Control (2.00 |
| Precision | mid and low assay controls across all | VCN/cell): 4% |
| | valid qualification assays must be ≤30%. | Low Control (0.02 |
| | | VCN/cell): 6% |
| Specificity | All replicate Ct values for the Mock T-cell | Transgene target: All |
| (Mock T-cell | DNA must be “Undetermined” for the | replicate Ct values |
| DNA) | Transgene target in addition having | were “Undetermined” |
| | mean hALB copies within 21,212-39,394 | in each assay. |
| | copies for each valid qualification | hALB target: mean |
| | assay. | hALB copies ranged |
| | | from 28,719-29,611. |
| Specificity | All replicates of the JNJ- | Transgene target: all |
| (JNJ-68284528 | 68284528 CAR-T DNA must | copy values were |
| CAR-T DNA) | have a quantifiable Transgene | quantifiable and ranged |
| | result in addition having mean | from 4,592.801- |
| | hALB copies within 21,212- | 5,153.907. |
| | 39,394 copies for each valid | hALB target: mean |
| | qualification assay. | hALB copies ranged |
| | | from 31,552-33,725. |
| Range | The range is defined as the copy range | Range: 193.939- |
| (Transgene | covered by the 5-point standard curve | 121212.121 copies |
| Target) | provided the Transgene target satisfies all | |
| | criteria for accuracy, linearity and | |
| | intermediate precision. | |
| Range (hALB | The range is defined as the copy range | Range: 121.212- |
| Target) | covered by the 5-point standard curve | 75757.576 copies |
| | provided the hALB target satisfies all | |
| | criteria for accuracy, linearity and | |
| | intermediate precision. | |
| LOQ (Transgene | LOQ is defined as the Transgene copy | LOQ: 0.02 VCN/cell |
| Target) | result for the lowest LOQ sample to have | LOQ sample Transgene |
| | % CV ≤20% for both the mean Transgene | copies of 303.030 |
| | copy result and mean VCN/cell results as | |
| | well as % recovery within 70-130% for | |
| | both the mean Transgene copy result and | |
| | mean VCN/cell result for each valid | |
| | qualification assay. | |
| LOQ (hALB | LOQ is defined as the copy value of | LOQ: 121.212 copies |
| Target) | Standard #5 provided the hALB | |
| | target satisfies all criteria for | |
| | accuracy, linearity and intermediate | |
| | precision. | |

7.0 Procedure
7.1 All assays were performed as described in the method qualification protocol. Only assays that met the assay acceptance criteria specified in the method were included in the evaluation of the method qualification acceptance criteria.

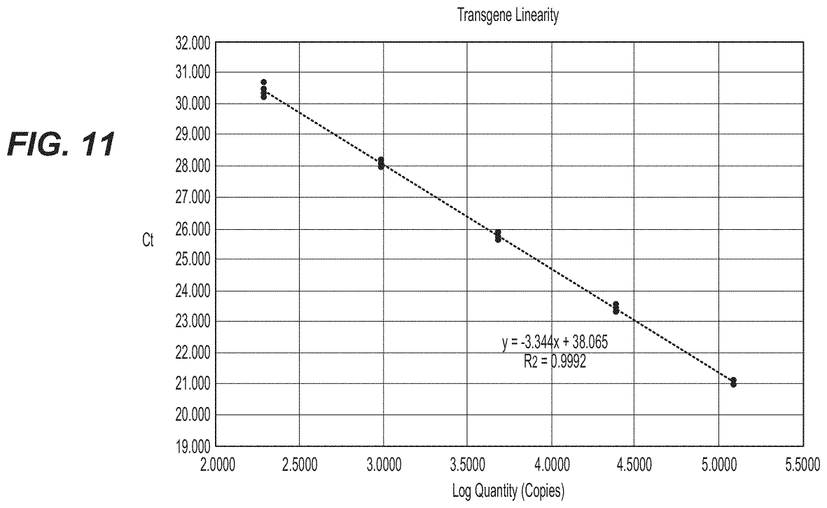

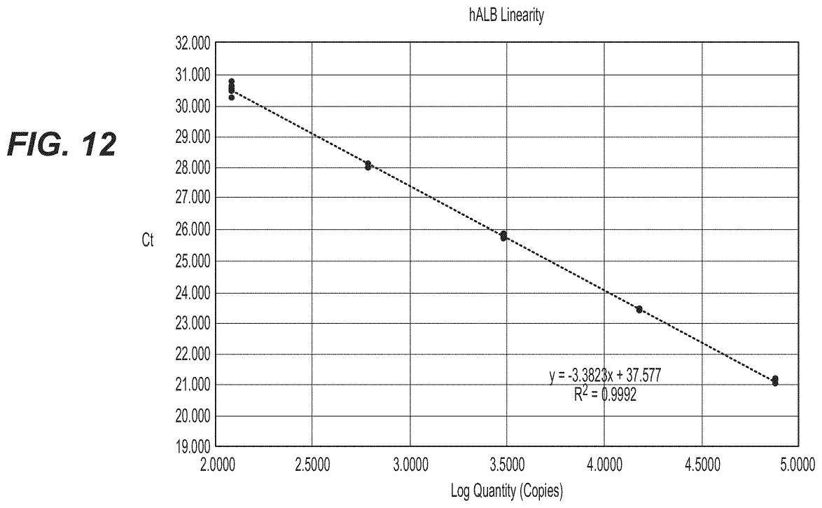

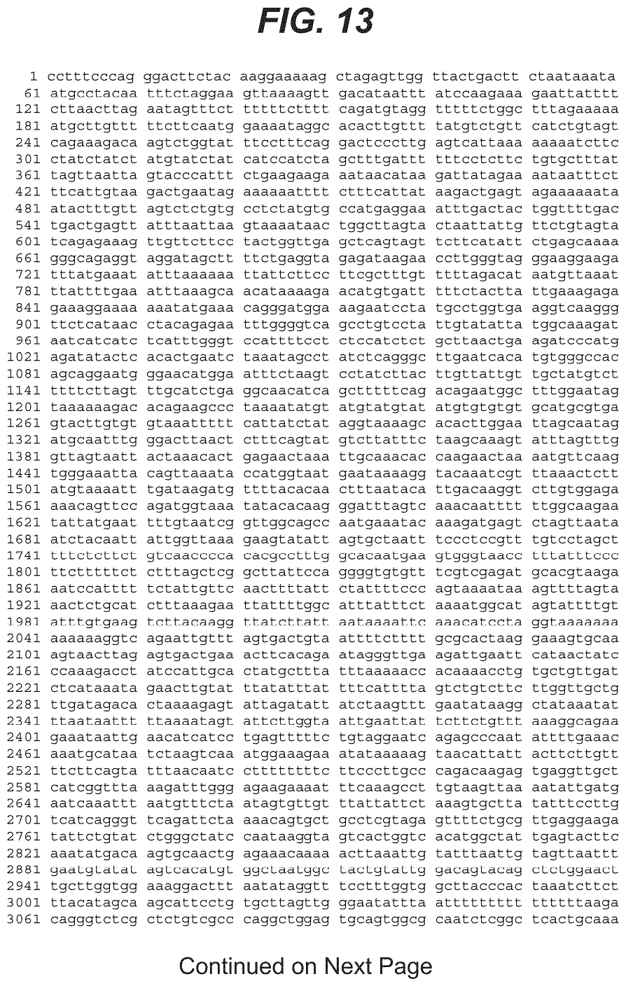

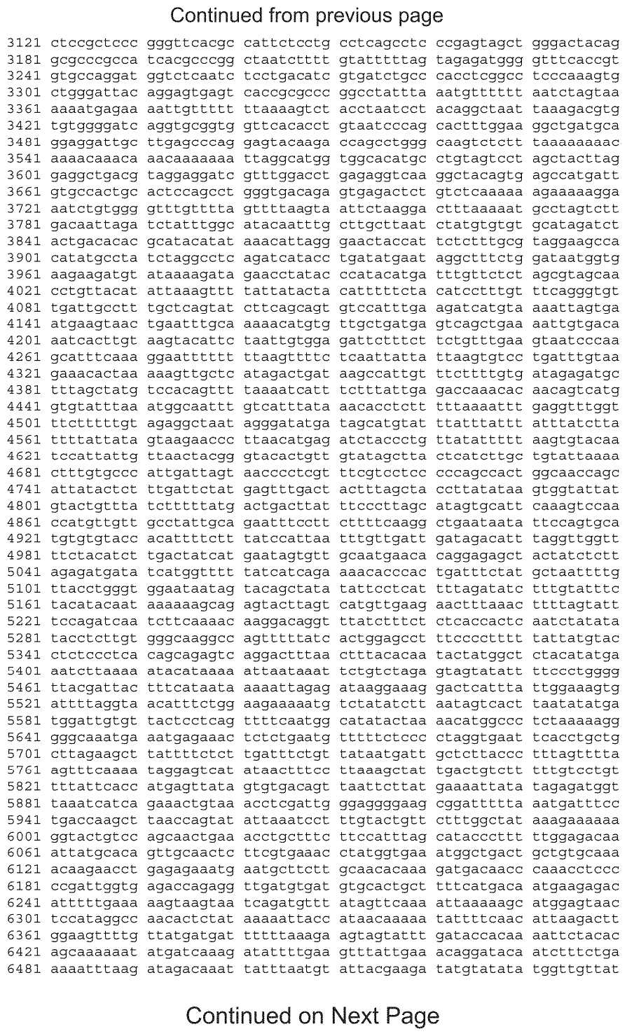

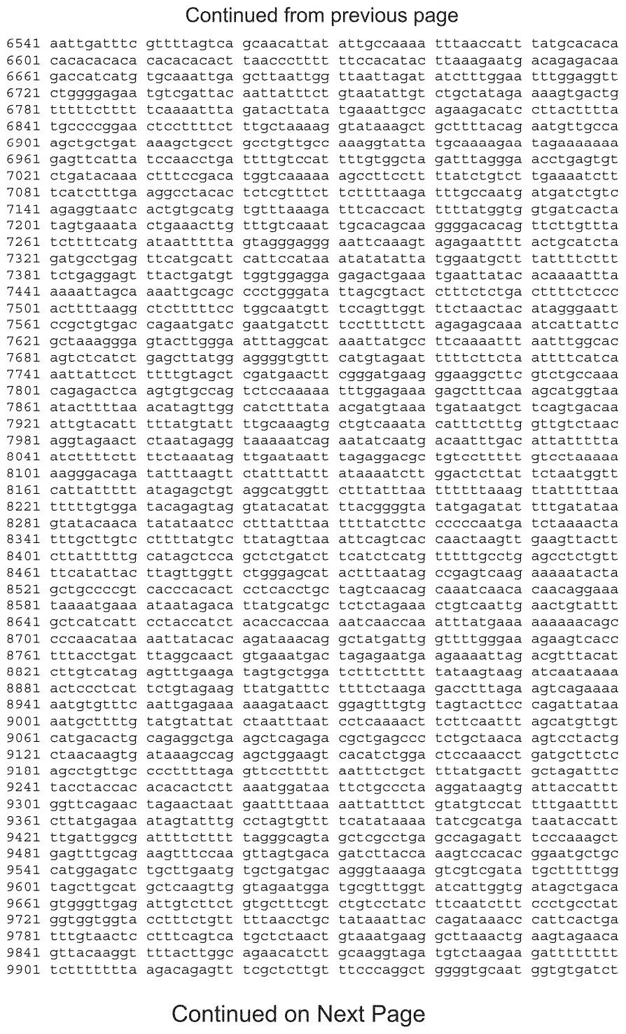

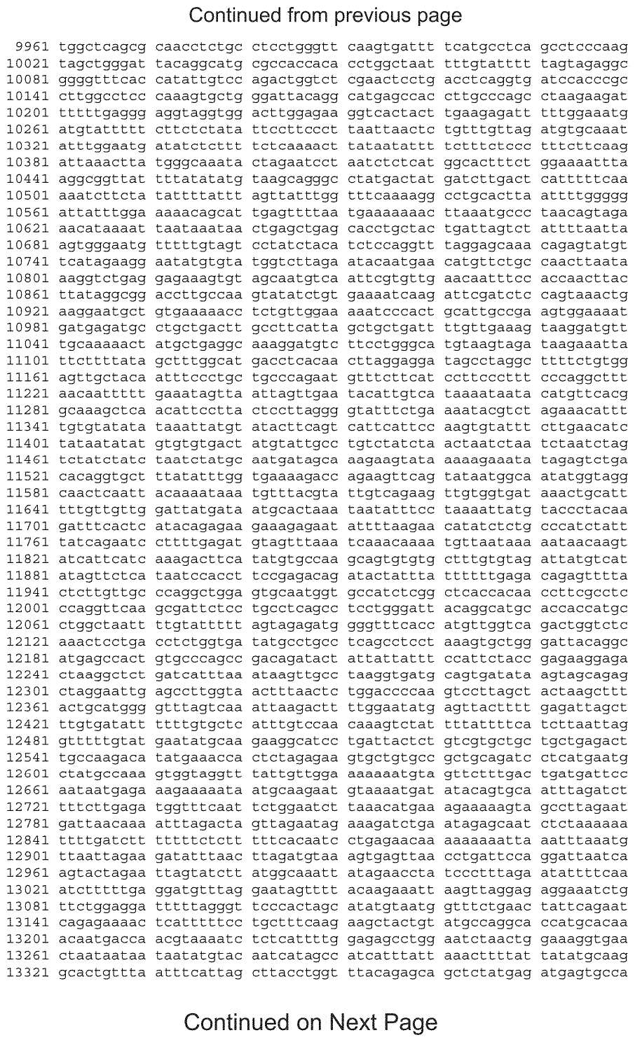

8.0 Results and Discussion
8.1 Standard Curve Linearity and Precision (Repeatability and Intermediate Precision)
   8.1.1 The 5-point standard curve results for all valid qualification assays for both the Transgene and hALB targets are summarized in Tables 13-14 and FIGS. 11-12. The R2 values of the Log 10 vs Ct values plots for both the transgene and hALB targets are 1.00. The repeatability of each standard ranged from 0.06-0.54% for the Transgene target and from 0.03-0.41% for the hALB target. The intermediate precision ranged from 0.26-0.47% for the transgene target and from 0.17-0.55% for the hALB target.

TABLE 13
Transgene Standard Curve Results

| | | | | | | Ct % CV |
|---|---|---|---|---|---|---|
| | | | Average | | Ct % CV | (Intermediate |
| | Assay # | Ct | Ct | Ct SD | (Repeatability) | Precision) |
| Standard | 1 | 20.987 | 21.064 | 0.067 | 0.32 | 0.26 |
| #1 | | 21.096 | | | | |
| | | 21.110 | | | | |
| | 2 | 20.980 | 21.025 | 0.042 | 0.20 | |
| | | 21.035 | | | | |
| | | 21.062 | | | | |
| | 3 | 20.966 | 20.979 | 0.012 | 0.06 | |
| | | 20.981 | | | | |
| | | 20.990 | | | | |
| Standard | 1 | 23.443 | 23.505 | 0.075 | 0.32 | 0.35 |
| #2 | | 23.485 | | | | |
| | | 23.589 | | | | |
| | 2 | 23.432 | 23.455 | 0.048 | 0.20 | |
| | | 23.422 | | | | |
| | | 23.510 | | | | |
| | 3 | 23.318 | 23.353 | 0.038 | 0.16 | |
| | | 23.348 | | | | |
| | | 23.393 | | | | |
| Standard | 1 | 25.923 | 25.853 | 0.062 | 0.24 | 0.30 |
| #3 | | 25.828 | | | | |
| | | 25.807 | | | | |
| | 2 | 25.822 | 25.782 | 0.037 | 0.14 | |
| | | 25.750 | | | | |
| | | 25.773 | | | | |
| | 3 | 25.741 | 25.701 | 0.036 | 0.14 | |
| | | 25.689 | | | | |
| | | 25.672 | | | | |
| Standard | 1 | 28.194 | 28.134 | 0.053 | 0.19 | 0.30 |
| #4 | | 28.092 | | | | |
| | | 28.117 | | | | |
| | 2 | 28.038 | 28.094 | 0.054 | 0.19 | |
| | | 28.098 | | | | |
| | | 28.146 | | | | |
| | 3 | 27.999 | 27.971 | 0.026 | 0.09 | |
| | | 27.948 | | | | |
| | | 27.966 | | | | |
| Standard | 1 | 30.336 | 30.490 | 0.165 | 0.54 | 0.47 |
| #5 | | 30.663 | | | | |
| | | 30.471 | | | | |
| | 2 | 30.201 | 30.270 | 0.081 | 0.27 | |
| | | 30.251 | | | | |
| | | 30.359 | | | | |
| | 3 | 30.487 | 30.426 | 0.103 | 0.34 | |
| | | 30.307 | | | | |
| | | 30.484 | | | | |
| Acceptance Criteria: | | | | | ≤30 | ≤30 |

TABLE 14
hALB Standard Curve Results

| | | | | | | Ct % CV |
|---|---|---|---|---|---|---|
| | | | Average | | Ct % CV | (Intermediate |
| | Assay # | Ct | Ct | Ct SD | (Repeatability) | Precision) |
| Standard | 1 | 21.038 | 21.060 | 0.022 | 0.11 | 0.26 |
| #1 | | 21.083 | | | | |
| | | 21.058 | | | | |
| | 2 | 21.080 | 21.147 | 0.059 | 0.28 | |
| | | 21.188 | | | | |
| | | 21.175 | | | | |
| | 3 | 21.081 | 21.063 | 0.017 | 0.08 | |
| | | 21.058 | | | | |
| | | 21.050 | | | | |
| Standard | 1 | 23.454 | 23.453 | 0.038 | 0.16 | 0.17 |
| #2 | | 23.415 | | | | |
| | | 23.491 | | | | |
| | 2 | 23.493 | 23.489 | 0.006 | 0.03 | |
| | | 23.482 | | | | |
| | | 23.493 | | | | |
| | 3 | 23.418 | 23.410 | 0.013 | 0.05 | |
| | | 23.418 | | | | |
| | | 23.396 | | | | |
| Standard | 1 | 25.802 | 25.772 | 0.032 | 0.13 | 0.21 |
| #3 | | 25.738 | | | | |
| | | 25.777 | | | | |
| | 2 | 25.898 | 25.827 | 0.072 | 0.28 | |
| | | 25.830 | | | | |
| | | 25.754 | | | | |
| | 3 | 25.783 | 25.758 | 0.042 | 0.16 | |
| | | 25.780 | | | | |
| | | 25.710 | | | | |
| Standard | 1 | 28.154 | 28.096 | 0.058 | 0.21 | 0.17 |
| #4 | | 28.095 | | | | |
| | | 28.039 | | | | |
| | 2 | 28.117 | 28.099 | 0.015 | 0.05 | |
| | | 28.089 | | | | |
| | | 28.092 | | | | |
| | 3 | 28.078 | 28.068 | 0.070 | 0.25 | |
| | | 27.994 | | | | |
| | | 28.133 | | | | |
| Standard | 1 | 30.498 | 30.569 | 0.072 | 0.23 | 0.55 |
| #5 | | 30.641 | | | | |
| | | 30.567 | | | | |
| | 2 | 30.804 | 30.773 | 0.066 | 0.21 | |
| | | 30.819 | | | | |
| | | 30.698 | | | | |
| | 3 | 30.508 | 30.434 | 0.126 | 0.41 | |
| | | 30.288 | | | | |
| | | 30.505 | | | | |
| Acceptance Criteria: | | | | | ≤30 | ≤30 |

8.2 QC Controls Accuracy and Precision (Repeatability and Intermediate Precision)
   8.2.1 The results for the 2.00 VCN/cell Mid Control and 0.20 VCN/cell Low Control are summarized in Table 15. The percent recovery of the average VCN/cell results for the Mid Control ranges from 93-95%.
      The percent recovery of the average VCN/cell results for the Low Control ranges from 79-84%. The repeatability for both the Mid and Low Controls ranges from 4-6%. The intermediate precision for the Mid and Low Controls is 4% and 6% respectively.

TABLE 15
2.00 VCN/cell Mid Control and 0.20 VCN/cell Low Control Results

| | | | | Avg | | | | % CV |
|---|---|---|---|---|---|---|---|---|
| | | Expected | Observed | Observed | VCN/cell | % CV | | (Intermediate |
| | Assay # | VCN/cell | VCN/cell | VCN/cell | SD | (Repeatability) | % Recovery | Precision) |
| Mid | 1 | 2.00 | 1.83 | 1.91 | 0.070 | 4 | 95 | 4 |
| Control | | | 1.95 | | | | | |
| | | | 1.94 | | | | | |
| | 2 | 2.00 | 1.80 | 1.90 | 0.081 | 4 | 95 | |
| | | | 1.92 | | | | | |
| | | | 1.96 | | | | | |
| | 3 | 2.00 | 1.72 | 1.85 | 0.116 | 6 | 93 | |
| | | | 1.94 | | | | | |
| | | | 1.89 | | | | | |
| Low | 1 | 0.20 | 0.16 | 0.17 | 0.010 | 6 | 84 | 6 |
| Control | | | 0.16 | | | | | |
| | | | 0.18 | | | | | |
| | 2 | 0.20 | 0.15 | 0.16 | 0.007 | 4 | 79 | |
| | | | 0.16 | | | | | |
| | | | 0.16 | | | | | |
| | 3 | 0.20 | 0.16 | 0.17 | 0.010 | 6 | 84 | |
| | | | 0.17 | | | | | |
| | | | 0.18 | | | | | |
| Acceptance Criteria: | | | | | | ≤30 | 70-130 | ≤30 |

8.3 Specificity
   8.3.1 The results for the mock T-cell DNA and CART DNA samples run for the assay specificity evaluation are summarized in Table 16. All replicates of the mock T-cell DNA sample were “Undetermined” for the transgene target. All replicates of the CAR T DNA had quantifiable transgene target copy results. In addition, both the mock T-cell DNA and CAR T DNA samples met the hALB sample acceptance criteria of hALB copies +/−30% of the expected 30,303.030 copies for 100 ng of DNA.

TABLE 16
Mock T-cell DNA and CAR T DNA Results

| | Assay | | Transgene | hALB | hALB | Avg hALB |
|---|---|---|---|---|---|---|
| | # | Transgene Ct | Copies | Ct | Copies | Copies |
| Mock T-cell | 1 | Undetermined | N/A | 22.476 | 28,874.957 | 29,226.484 |
| DNA | | Undetermined | N/A | 22.474 | 28,918.598 | |
| | | Undetermined | N/A | 22.426 | 29,885.896 | |
| | 2 | Undetermined | N/A | 22.505 | 29,269.688 | 29,610.689 |
| | | Undetermined | N/A | 22.491 | 29,537.838 | |
| | | Undetermined | N/A | 22.467 | 30,024.545 | |
| | 3 | Undetermined | N/A | 22.487 | 28,520.166 | 28,718.760 |
| | | Undetermined | N/A | 22.489 | 28,481.504 | |
| | | Undetermined | N/A | 22.455 | 29,154.613 | |
| Acceptance Criteria: | | Undetermined | | | | 21,212- |
| | | | | | | 39,394 |
| JNJ- | 1 | 25.796 | 4,894.034 | 22.321 | 32,078.729 | 31,552.141 |
| 68284528 | | 25.831 | 4,776.155 | 22.356 | 31,328.813 | |
| CAR-T | | 25.830 | 4,779.097 | 22.360 | 31,248.881 | |
| DNA | 2 | 25.742 | 4,792.772 | 22.353 | 32,438.480 | 33,725.105 |
| | | 25.783 | 4,656.793 | 22.230 | 35,245.887 | |
| | | 25.803 | 4,592.801 | 22.305 | 33,490.949 | |
| | 3 | 25.719 | 4,739.884 | 22.373 | 30,857.877 | 32,056.623 |
| | | 25.597 | 5,153.907 | 22.263 | 33,273.906 | |
| | | 25.742 | 4,666.396 | 22.318 | 32,038.086 | |
| Acceptance Criteria: | | | Quantifiable | | | 21,212- |
| | | | result | | | 39,394 |

8.4 Range and LOQ
   8.4.1 Both the transgene and hALB targets met all acceptance criteria for accuracy, linearity, and intermediate precision. Therefore, the range for both the transgene and hALB targets are defined as the copy range of the 5-point standard curve. The transgene range is 193.939-121212.121 copies. The hALB range is 121.212-75757.576 copies. In addition, the LOQ for the hALB target is defined as 121.212 copies.
   8.4.2 The results for the 0.014 VCN/cell and 0.02 VCN/cell LOQ samples are summarized in Table 17. At least one of the triplicate Ct values for the 0.014 VCN/cell LOQ sample did not fall within the Ct range of the transgene standard curve in each valid qualification assay. Therefore, the transgene copy values could not be accurately determined and the LOQ criteria was unable to be evaluated. However, the 0.02 VCN/cell LOQ sample resulted in a % recovery of 73-80%. The 0.02 VCN/cell LOQ sample also resulted in a % CV of the mean transgene copy values and mean VCN/cell results of 1-9% and 2-11% respectively. The transgene target LOQ is therefore defined as the expected transgene copy value of the 0.02 VCN/cell LOQ sample of 303.030 copies.

TABLE 17
0.014 VCN/cell and 0.02 VCN/cell LOQ Samples Results

| | | | Observed | | Observed | Transgene | | | | |
|---|---|---|---|---|---|---|---|---|---|---|
| | | Observed | Transgene | Observed | Transgene | Quantity | Observed | Observed | Observed | |
| | Assay | Transgene | Avg | Transgene | Copies % | % | Avg | VCN/cell | VCN/cell % | VCN/cell % |
| | # | Copies | Copies | SD Copies | CV | Recovery | VCN/cell | SD | CV | Recovery |
| 0.014 | 1 | N/A | N/A | N/A | N/A | N/A | N/A | N/A | N/A | N/A |
| VCN/ | | 177.993 | | | | | | | | |
| cell | | N/A | | | | | | | | |
| (212.121 | 2 | N/A | N/A | N/A | N/A | N/A | N/A | N/A | N/A | N/A |
| Transgene | | N/A | | | | | | | | |
| Copies) | | N/A | | | | | | | | |
| | 3 | 187.975 | N/A | N/A | N/A | N/A | N/A | N/A | N/A | N/A |
| | | N/A | | | | | | | | |
| | | 188.407 | | | | | | | | |

Quantity values that are N/A were not quantifiable due to the Ct value being higher than the highest Standard #5 Ct
value (i.e. Outside the Ct range of the Transgene standard curve)

| | | | | | | | | | | |
|---|---|---|---|---|---|---|---|---|---|---|
| 0.02 | 1 | 237.648 | 236.475 | 5.320 | 2 | 78 | 0.016 | 0.0002 | 2 | 80 |
| VCN/ | | 241.111 | | | | | | | | |
| cell | | 230.666 | | | | | | | | |
| (303.030 | 2 | 212.253 | 212.187 | 2.581 | 1 | 70 | 0.015 | 0.0003 | 2 | 73 |
| Transgene | | 214.734 | | | | | | | | |
| Copies | | 209.573 | | | | | | | | |
| ) | 3 | 221.846 | 221.701 | 20.836 | 9 | 73 | 0.015 | 0.0016 | 11 | 75 |
| | | 200.792 | | | | | | | | |
| | | 242.464 | | | | | | | | |
| Acceptance Criteria: | | | | | ≤20 | 30-170 | | | ≤20 | 30-170 |

The teachings of all patents, published applications, and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A probe and primer set comprising: a probe comprising a nucleotide sequence of SEQ ID NO: 10 and at least one label attached to the probe; a first primer comprising a nucleic acid sequence of SEQ ID NO: 11; and a second primer comprising a nucleic acid sequence of SEQ ID NO: 12.

2. The probe and primer set of claim 1, wherein the at least one label comprises a radioactive isotope, an enzyme substrate, a chemiluminescent agent, a fluorophore, a fluorescence quencher, an enzyme, a chemical, or a combination thereof.

3. A kit for quantitating transgene integration into a chimeric antigen receptor (CAR) T cell, comprising: a probe comprising a nucleotide sequence of SEQ ID NO: 10 and at least one label attached to the probe; a first primer comprising a nucleic acid sequence of SEQ ID NO: 11; and a second primer comprising a nucleic acid sequence of SEQ ID NO: 12.

4. The kit of claim 3, wherein the at least one label attached to the probe comprises a radioactive isotope, an enzyme substrate, a chemiluminescent agent, a fluorophore, a fluorescence quencher, an enzyme, a chemical, or a combination thereof.

5. The kit of claim 3, wherein the kit comprises an array that comprises the probe.

6. The kit of claim 5, wherein the array is a multi-well plate.

7. The kit of claim 3, wherein the kit further comprises a human albumin (hALB) probe comprising a nucleic acid sequence of SEQ ID NO: 22 and at least one label attached to the hALB probe, a first hALB primer comprising a nucleic acid sequence of SEQ ID NO: 23, and a second hALB primer comprising a nucleic acid sequence of SEQ ID NO: 24.

8. The kit of claim 7, wherein the at least one label attached to the hALB probe comprises a radioactive isotope, an enzyme substrate, a chemiluminescent agent, a fluorophore, a fluorescence quencher, an enzyme, a chemical, or a combination thereof.

9. The kit of claim 3, wherein the kit further comprises a reference gene probe and at least one label attached to the reference gene probe, a first reference gene primer, and a second reference gene primer.

10. A method for quantitating transgene integration into a chimeric antigen receptor (CAR) T cell, comprising:
amplifying nucleic acids from the CAR T cell with a first CAR primer comprising a nucleic acid sequence of SEQ ID NO: 11 and a second CAR primer comprising a nucleic acid sequence of SEQ ID NO: 12, thereby generating amplified CAR nucleic acids;
amplifying the nucleic acids from the CAR T cell with a first hALB primer comprising a nucleic acid sequence of SEQ ID NO: 23 and a second hALB primer comprising a nucleic acid sequence of SEQ ID NO: 24, thereby generating amplified hALB nucleic acids;
detecting hybridization between the amplified CAR nucleic acids and a CAR probe comprising a nucleotide sequence of SEQ ID NO: 10 via a target signal from at least one label attached to the CAR probe;
detecting hybridization between the amplified hALB nucleic acids and the hALB probe comprising a nucleotide sequence of SEQ ID NO: 22 via a reference signal from at least one label attached to the hALB probe; and
quantitating transgene copy number by comparison of the target signal relative to the reference signal.

11. The method of claim 10, wherein detecting hybridization among the amplified CAR nucleic acids and the CAR probe comprises detecting a change in target signal from the at least one label attached to the CAR probe during or after hybridization relative to a target signal from the label attached to the CAR probe before hybridization.

12. The method of claim 10, wherein the amplifying comprises polymerase chain reaction (PCR).

13. The method of claim 12, wherein the PCR is real-time PCR, reverse transcriptase-polymerase chain reaction (RT-PCR), real-time reverse transcriptase-polymerase chain reaction (rt RT-PCR), digital PCR (dPCR), ligase chain reaction, or transcription-mediated amplification (TMA).

14. The method of claim 10, wherein at least one label attached to the CAR probe comprises a fluorophore.

15. The method of claim 10, wherein at least one label attached to the hALB probe comprises a fluorophore.

16. A method for quantitating transgene integration into a chimeric antigen receptor (CAR) T cell, comprising:
contacting nucleic acids from the CAR T cell with a first CAR primer, a second CAR primer, a first hALB primer and a second hALB primer, wherein the first CAR primer comprises a nucleic acid sequence of SEQ ID NO: 11, the second CAR primer comprises a nucleic acid sequence of SEQ ID NO: 12, the first hALB primer comprises a nucleic acid sequence of SEQ ID NO: 23 and the second hALB primer comprises a nucleic acid sequence of SEQ ID NO: 24;
amplifying the CAR nucleic acids with the first CAR primer and second CAR primer, thereby generating amplified CAR nucleic acids;
amplifying hALB nucleic acids with the first hALB primer and second hALB primer, thereby generating amplified hALB nucleic acids;
detecting hybridization between the amplified CAR nucleic acids and a CAR probe comprising a nucleotide sequence of SEQ ID NO: 10 via a target signal from at least one label attached to the CAR probe;
detecting hybridization between the amplified hALB nucleic acids and the hALB probe via a reference signal from at least one label attached to the hALB probe; and
quantitating transgene copy number by comparison of the target signal relative to the reference signal.

17. The method of claim 16, wherein detecting hybridization among the amplified hALB nucleic acid molecules and the hALB probe comprises detecting a change in target signal from the at least one label attached to the hALB probe during or after hybridization relative to a target signal from the label attached to the hALB probe before hybridization.

18. The method of claim 16, wherein the amplifying comprises polymerase chain reaction (PCR).

19. The method of claim 18, wherein the PCR is real-time PCR, reverse transcriptase-polymerase chain reaction (RT-PCR), real-time reverse transcriptase-polymerase chain reaction (rt RT-PCR), digital PCR (dPCR), ligase chain reaction, or transcription-mediated amplification (TMA).

20. A method for quantitating transgene integration into a chimeric antigen receptor (CAR) T cell, comprising:
amplifying nucleic acids from the CAR T cell with a first CAR primer comprising a nucleic acid sequence of SEQ ID NO: 11 and a second CAR primer comprising a nucleic acid sequence of SEQ ID NO: 12, thereby generating amplified CAR nucleic acids;
amplifying the nucleic acids from the CAR T cell with a first reference gene primer and a second reference gene primer, thereby generating amplified reference gene nucleic acids;
detecting hybridization between the amplified CAR nucleic acids and a CAR probe comprising a nucleotide sequence of SEQ ID NO: 10 via a target signal from at least one label attached to the CAR probe;
detecting hybridization between the amplified reference gene nucleic acids and the reference gene probe via a reference signal from at least one label attached to the reference gene probe; and
quantitating transgene copy number by comparison of the target signal relative to the reference signal.

21. The method of claim 20, wherein detecting hybridization among the amplified CAR nucleic acids and the CAR probe comprises detecting a change in target signal from the at least one label attached to the CAR probe during or after hybridization relative to a target signal from the label attached to the CAR probe before hybridization.

22. The method of claim 20, wherein detecting hybridization among the amplified reference gene nucleic acid molecules and the reference gene probe comprises detecting a change in target signal from the at least one label attached to the reference gene probe during or after hybridization relative to a target signal from the label attached to the reference gene probe before hybridization.

23. The method of claim 20, wherein the amplifying comprises polymerase chain reaction (PCR).

24. The method of claim 23, wherein the PCR is real-time PCR, reverse transcriptase-polymerase chain reaction (RT-PCR), real-time reverse transcriptase-polymerase chain reaction (rt RT-PCR), digital PCR (dPCR), ligase chain reaction, or transcription-mediated amplification (TMA).

25. The method of claim 20, wherein at least one label attached to the CAR probe comprises a fluorophore.

26. The method of claim 20, wherein at least one label attached to the reference gene probe comprises a fluorophore.

27. A method for quantitating transgene integration into a chimeric antigen receptor (CAR) T cell, comprising:
contacting nucleic acids from the CAR T cell with a first CAR primer, a second CAR primer, a first reference gene primer and a second reference gene primer, wherein the first CAR primer comprises a nucleic acid sequence of SEQ ID NO: 11 and the second CAR primer comprises a nucleic acid sequence of SEQ ID NO: 12;
amplifying the CAR nucleic acids with the first CAR primer and the second CAR primer, thereby generating amplified CAR nucleic acids;
amplifying reference gene nucleic acids with the first reference gene primer and second reference gene primer, thereby generating amplified reference gene nucleic acids;
detecting hybridization between the amplified CAR nucleic acids and a CAR probe comprising a nucleotide sequence of SEQ ID NO: 10 via a target signal from at least one label attached to the CAR probe;
detecting hybridization between the amplified reference gene nucleic acids and the reference gene probe via a reference signal from at least one label attached to the reference gene probe; and
quantitating transgene copy number by comparison of the target signal relative to the reference signal.

28. The method of claim 27, wherein detecting hybridization among the amplified CAR nucleic acids and the CAR probe comprises detecting a change in target signal from the at least one label attached to the CAR probe during or after hybridization relative to a target signal from the label attached to the CAR probe before hybridization.

29. The method of claim 27, wherein detecting hybridization among the amplified reference gene nucleic acid molecules and the reference gene probe comprises detecting a change in target signal from the at least one label attached to the reference gene probe during or after hybridization relative to a target signal from the label attached to the reference gene probe before hybridization.

30. The method of claim 27 wherein the amplifying comprises polymerase chain reaction (PCR).

31. A method of generating a chimeric antigen receptor (CAR) T cell, comprising: introducing a CAR transgene into a T cell to obtain a transgene integrated T cell;
determining CAR transgene integration, comprising:
amplifying nucleic acids from the transgene integrated T cell with a first CAR primer comprising a nucleic acid sequence of SEQ ID NO: 11 and a second CAR primer comprising a nucleic acid sequence of SEQ ID NO: 12, thereby generating amplified CAR nucleic acids;
amplifying the nucleic acids from the transgene integrated T cell with a first reference gene primer and a second reference gene primer, thereby generating amplified reference gene nucleic acids;
detecting hybridization between the amplified CAR nucleic acids and a CAR probe comprising a nucleotide sequence of SEQ ID NO: 10 via a target signal from at least one label attached to the CAR probe;
detecting hybridization between the amplified reference gene nucleic acids and the reference gene probe via a reference signal from at least one label attached to the reference gene probe; and
quantitating transgene copy number by comparison of the target signal relative to the reference signal; and
obtaining a CAR T cell comprising at least one copy of the integrated CAR transgene.

32. The method of claim 31, wherein detecting hybridization among the amplified CAR nucleic acids and the CAR probe comprises detecting a change in target signal from the at least one label attached to the CAR probe during or after hybridization relative to a target signal from the label attached to the CAR probe before hybridization.

33. The method of claim 31, wherein detecting hybridization among the amplified reference gene nucleic acid molecules and the reference gene probe comprises detecting a change in target signal from the at least one label attached to the reference gene probe during or after hybridization relative to a target signal from the label attached to the reference gene probe before hybridization.

34. The method of claim 31, wherein the amplifying comprises polymerase chain reaction (PCR).

35. The method of claim 34, wherein the PCR is real-time PCR, reverse transcriptase-polymerase chain reaction (RT-PCR), real-time reverse transcriptase-polymerase chain reaction (rt RT-PCR), digital PCR (dPCR), ligase chain reaction, or transcription-mediated amplification (TMA).

36. The method of claim 31, wherein at least one label attached to the CAR probe comprises a fluorophore.

37. The method of claim 31, wherein at least one label attached to the reference gene probe comprises a fluorophore.

38. A CART cell generated by the method of claim 20.
